(19) United States
(12) Patent Application Publication
(10) Pub. No.: US 2026/0345862 A1
(43) Pub. Date: Nov. 5, 2026

(54) FORMULATIONS FOR USE IN ADDITIVE MANUFACTURING, CONTAINING A TETRACYCLINE COMPOUND AS A LIGHT-ABSORBING SUBSTANCE

(71) Applicant: CollPlant Ltd., Rehovot (IL)

(72) Inventors: Roi MASHIACH, Ramle (IL); Miriam STERN, ModiIn (IL); Gal SALINAS, Gedera (IL); Sivan LOUZOUN ZADA, Givatayim (IL); Shirel COHEN, Tel-Aviv (IL); Tami WINITZ YEHEZKEL, Ness Ziona (IL)

(73) Assignee: CollPlant Ltd., Rehovot (IL)

(21) Appl. No.: 19/124,327

(22) PCT Filed: Oct. 27, 2023
(86) PCT No.: PCT/IL2023/051112

§ 371 Date: Apr. 25, 2025

Related U.S. Application Data
(60) Provisional application No. 63/419,792, filed on Oct. 27, 2022.

(51) Int. Cl. A61L 27/24; B29C 64/124; B33Y 10/00; B33Y 70/10; B33Y 80/00

(52) U.S. Cl. CPC A61L 27/24; B29C 64/124; B33Y 10/00; B33Y 70/10; B33Y 80/00; B29K 2689/00

(57) ABSTRACT

Formulations usable for additive manufacturing of a three-dimensional object featuring, in at least a portion thereof, a biological and/or biocompatible material, which utilize minocycline and/or any other compound that features a tetracycline skeleton, as a photoabsorber, are provided, as well as additive manufacturing processes utilizing same and objects obtainable thereby.

### RELATED APPLICATION/S

This application claims the benefit of priority under 35 USC § 119(e) of U.S. Provisional Patent Application No. 63/419,792 filed on Oct. 27, 2022, the contents of which are incorporated herein by reference in their entirety.

This application is related to International Patent Application No. PCT/IL2022/051143, which was co-filed with U.S. Provisional Patent Application No. 63/419,792 on Oct. 27, 2022, the contents of which are incorporated herein by reference in their entirety.

This application is also related to co-filed U.S. Provisional Patent Application having Attorney's Docket No. 97733, entitled “METHODS OF IDENTIFYING LIGHT-ABSORBING SUBSTANCES SUITABLE FOR USE IN ADDITIVE MANUFACTURING AND LIGHT-ABSORBING SUBSTANCES IDENTIFIED THEREBY”, the content of which are incorporated herein by reference in their entirety.

### SEQUENCE LISTING STATEMENT

The XML file, entitled 97732 Sequence Listing.xml, created on 27 Oct. 2023, comprising 57,344 bytes, submitted concurrently with the filing of this application is incorporated herein by reference.

### FIELD AND BACKGROUND OF THE INVENTION

The present invention, in some embodiments thereof, relates to additive manufacturing, and more particularly, but not exclusively, to formulations usable in additive manufacturing of 3D objects featuring a biological material in at least a portion thereof and to additive manufacturing processes employing same.

Additive manufacturing (AM) is generally a process in which a three-dimensional (3D) object is manufactured utilizing a computer model of the objects. The basic operation of any AM system consists of slicing a three-dimensional computer model into thin cross sections, translating the result into two-dimensional position data and feeding the data to control equipment which manufacture a three-dimensional structure in a layerwise manner.

Various AM technologies exist, amongst which are stereolithography, digital light processing (DLP), and three-dimensional (3D) printing such as 3D inkjet printing. Such techniques are generally performed by layer-by-layer deposition and hardening (e.g., solidification) of one or more building materials, which typically include photopolymerizable (photocurable) materials.

Stereolithography, for example, is an additive manufacturing process which employs a liquid ultraviolet (UV)-curable building material and a UV laser. In such a process, for each dispensed layer of the building material, the laser beam traces a cross-section of the part pattern on the surface of the dispensed liquid building material. Exposure to the UV laser light cures and solidifies the pattern traced on the building material and joins it to the layer below. After being built, the formed parts are immersed in a chemical bath in order to be cleaned of excess building material and are subsequently cured in an UV oven.

In three-dimensional printing processes, for example, a building material is dispensed from a dispensing head having a set of nozzles or nozzle arrays to deposit layers on a receiving substrate. Depending on the building material, the layers may then be cured or solidify using a suitable device.

The building materials may include modeling material formulation(s) and support material formulation(s), which form, upon hardening, the object and the temporary support constructions supporting the object as it is being built, respectively. The modeling material formulation(s) is/are deposited to produce the desired object and the support material formulation(s) is/are used, with or without modeling material elements, to provide support structures for specific areas of the object during building and assure adequate vertical placement of subsequent object layers, e.g., in cases where objects include overhanging features or shapes such as curved geometries, negative angles, voids, and so on.

Both the modeling and support material formulations typically feature a viscosity that allows dispensing/depositing, and upon being dispensed and optionally exposed to curing/hardening, feature a higher viscosity. Both the modeling and support materials are preferably liquid at the working temperature at which they are dispensed, and are subsequently hardened, typically upon exposure to hardening or curing condition such as curing energy (e.g., UV curing), to form the required layer shape. After printing completion, support structures, if present, are removed to reveal the final shape of the fabricated 3D object. The hardening (curing) of the dispensed materials typically involves polymerization (e.g., photopolymerization) and/or crosslinking (e.g., photocrosslinking).

Additive manufacturing has been first used in biological applications for forming three-dimensional sacrificial resin molds in which 3D scaffolds from biological materials were created.

3D bioprinting is an additive manufacturing methodology which uses biological materials, optionally in combination with chemicals and/or cells, that are printed layer-by-layer with a precise positioning and a tight control of functional components placement to create a 3D structure.

Three dimensional (3D) bioprinting is gaining momentum in many medicinal applications, especially in regenerative medicine, to address the need for complex scaffolds, tissues and organs suitable for transplantation.

Inherent to 3D printing in general is that the mechanical properties of the printing media (the dispensed building material) are very different from the post-printed cured (hardened) material.

To allow tight control on the curing (e.g., polymerization) after printing, the building material commonly includes polymerizable (e.g., photopolymerizable) moieties or groups that polymerize (e.g., by chain elongation and/or cross-linking) upon being dispensed, to preserve the geometric shape and provide the necessary physical properties of the final product.

Different technologies have been developed for 3D bioprinting, including 3D Inkjet printing, Extrusion printing, Laser-assisted printing, digital light processing, and Projection stereolithography [see, for example, Murphy S V, Atala A, Nature Biotechnology. 2014 32 (8); Miller J S, Burdick J. ACS Biomater. Sci. Eng. 2016, 2, 1658-1661]. Each technology has its different requirements for the dispensed building material (also referred to herein as printing media), which is derived from the specific application mechanism and the curing/gelation process required to maintain the 3D structure of the scaffold post printing.

Most of the 3D bioprinting technologies employ photopolymerizable materials and electromagnetic irradiation as the curing condition to which the dispensed material is exposed. The printing media in such technologies typically include a photoinitiator, which is capable of at least partially penetrating into voids between the hardened material, and is also capable of spreading onto the exterior surface of the hardened material. The photo-initiator is responsible for the initiation of the polymerization process when it is exposed to light with the right wavelength and energy level.

Exposing to electromagnetic irradiation is sometimes associated with selectivity issues, due to absorbance of the light energy by non-patterned building materials, which may lead to inaccurate parts, shapes and/or rough part edges. One way to overcome these limitations is the use of a photoabsorber, or a photoblocker, which is also referred to herein as a dye substance, which is capable of absorbing light at a respective wavelength range, and thereby improve the resolution and achieve defined porosity and channels of scaffolds. The photoabsorber is typically responsible for reducing the light penetration mainly in the Z axis (through the printed layers) but also increases the required energy levels for the polymerization process in XY plane.

Photoabsorbers usable in additive manufacturing in general and in 3D bioprinting in particular, are described, for example, in Zhang et al., Burns Trauma. 2022; 10: tkac010; WO 2022/093236; U.S. Patent Application Publication Nos. 2020/339925 and 2021/229364; U.S. Pat. No. 10,597,289; and CN 114958079.

Additional Background Art includes U.S. Patent Application Publication No. 2017/143831; WO 2018/225076; U.S. Patent application Publication No. 2018/0193524; WO 2015/032985; Drzewiecki et al. (2014) Langmuir, 30(37), 11204-11211; Ravichandran et al. (2015) Journal of Materials Chemistry B, 4(2), 318-326; Gaudet & Shreiber (2012) Biointerphases, 7(1), 25; and co-filed PCT International Patent Application PCT/IL2022/051143 (published as WO 2023/073711).

### SUMMARY OF THE INVENTION

According to an aspect of some embodiments of the present invention there is provided a curable formulation for use, or usable, in additive manufacturing (e.g., DLP) of a three-dimensional object, the formulation comprising a photoinitiator, a photocurable biological or biocompatible material and a dye substance capable of absorbing light at a wavelength of from 300 nm to 800 nm (a photoabsorber), the dye substance comprising minocycline.

According to an aspect of some embodiments of the present invention there is provided a curable formulation for use, or usable, in additive manufacturing (e.g., DLP) of a three-dimensional object, the formulation comprising a photoinitiator, a photocurable biological or biocompatible material and a dye substance capable of absorbing light at a wavelength of from 300 nm to 800 nm (a photoabsorber), the dye substance comprising a compound featuring a tetracycline skeleton (also referred to herein interchangeably as a tetracycline compound), as defined herein.

According to some of any of the embodiments described herein, an amount of the minocycline or of the compound featuring a tetracycline skeleton ranges from about 0.01 to about 5% by weight of the total weight of the formulation.

According to some of any of the embodiments described herein, an amount of the photoinitiator ranges from about 0.1 to about 1% by weight of the total weight of the formulation.

According to some of any of the embodiments described herein, the photocurable biocompatible material comprises a collagen that features a plurality of photocurable groups.

According to some of any of the embodiments described herein, the photocurable groups comprise (meth)acrylic groups.

According to some of any of the embodiments described herein, the collagen is a human Type I collagen.

According to some of any of the embodiments described herein, the collagen is a recombinant collagen.

According to some of any of the embodiments described herein, the collagen is a plant-derived recombinant collagen.

According to some of any of the embodiments described herein, the collagen is a plant-derived recombinant human Type I collagen.

According to some of any of the embodiments described herein, the curable formulation further comprises an additional (e.g., biocompatible) curable material that features a plurality of photocurable groups.

According to an aspect of some embodiments of the present invention there is provided a process (or method) of additive manufacturing a three-dimensional object featuring, in at least a portion thereof, a biological or a biocompatible material, the process comprising dispensing at least one modeling material formulation to sequentially form a plurality of layers in a configured pattern corresponding to a shape of the object, wherein for at least a portion of the layers, the dispensing is of a modeling material formulation that comprises the curable formulation as described herein in any of the respective embodiments and any combination thereof, thereby manufacturing the three-dimensional object.

According to some of any of the embodiments described herein, the process further comprises exposing the portion of the layers to irradiation suitable for hardening the bioink composition.

According to some of any of the embodiments described herein, the exposing is for time period that ranges from about 1 second to about 120 second.

According to some of any of the embodiments described herein, the irradiation is at wavelength within the UV-vis range.

According to some of any of the embodiments described herein, the irradiation is at level that ranges from about 1 to about 150, or from about 1 to about 130, or from about 1 to about 100 mW/cm².

According to some of any of the embodiments described herein, the additive manufacturing is DLP.

According to an aspect of some embodiments of the present invention there is provided a process of additive manufacturing a three-dimensional object featuring, in at least a portion thereof, a biological or a biocompatible material, the process comprising sequentially exposing, in a layerwise manner, a modeling material formulation to a curing condition suitable for hardening the curable formulation, the exposing being in a configured pattern corresponding to the shape of the object, wherein the modeling material formulation comprises the curable formulation as described herein in any of the respective embodiments and any combination thereof, thereby manufacturing the three-dimensional object.

According to some of any of the embodiments described herein, the exposing is for a time period that ranges from 1 second to 120 second for each layer.

According to some of any of the embodiments described herein, the curing condition comprises irradiation.

According to some of any of the embodiments described herein, the irradiation is at a wavelength within the UV-vis range.

According to some of any of the embodiments described herein, the irradiation is at a wavelength that ranges from 300 to 800 nm, or from 300 to 600 nm, or from 300 to 500 nm, or from 350 to 450 nm.

According to some of any of the embodiments described herein, the irradiation is at level that ranges from 1 to 150 mW/cm². According to some of any of the embodiments described herein, the curable formulation described above contains a compound featuring a tetracycline skeleton. In these embodiments, the amount of the tetracycline compound ranges from about 0.01 to about 5% by weight of the total formulation. The compound featuring a tetracycline skeleton, as described herein in any of the respective embodiments, for example, in Formula X. In certain embodiments consistent with the above, Y within the tetracycline compound may be amine. In various embodiments described herein, Y within the tetracycline compound may be amine.

In some embodiments, R₂ and R₃ within the tetracycline compound can be each hydrogen. In certain embodiments, the compound featuring a tetracycline skeleton, as per the above, can include tetracycline and its various derivatives, including dihydrosteffimycin, demethyltetracycline. In some embodiments, the curable formulation, which includes a compound featuring a tetracycline skeleton wherein said compound comprise tetracycline derivatives comprising oxytetracycline, lymcycline, chlorotetracycline. In accordance with these embodiments, the curable formulation, which includes a compound featuring a tetracycline skeleton as described herein, comprises tetracycline derivatives including minocycline, doxycycline, tetracycline, or their pharmaceutically acceptable salts. In certain embodiments, the salt of minocycline in the curable formulation can be minocycline hydrochloride. In some embodiments, the salt of tetracycline in the curable formulation can be tetracycline hydrochloride.

In various embodiments, the salt of doxycycline in the curable formulation can be doxycycline hyclate. In certain embodiments, the curable formulation may include an aqueous carrier. In some embodiments, the aqueous solution in the curable formulation may be selected from a neutral aqueous solution or an acidic aqueous solution. In certain embodiments, the amount of the photoinitiator in the curable formulation may range from 0.01 to 1% by weight of the total formulation. In some embodiments, the amount of the photoinitiator in the curable formulation may range from 0.1 to 1% by weight of the total formulation. In various embodiments, the amount of the photoinitiator in the curable formulation may range from 0.01 to 5% by weight of the total formulation. In certain embodiments, the amount of the photoinitiator in the curable formulation may range from 0.01 to 1% by weight of the total formulation. In some embodiments, the photocurable biocompatible material in the curable formulation may comprise a collagen that features a plurality of photocurable groups. In certain embodiments, the photocurable groups in the curable formulation may comprise (meth)acrylic groups. In some embodiments, the collagen in the curable formulation may be human Type I collagen. In various embodiments, the collagen in the curable formulation may be a recombinant collagen. In certain embodiments, the collagen in the curable formulation may be a plant-derived recombinant collagen. In some embodiments, the plant-derived recombinant collagen in the curable formulation may be human Type I collagen. In certain embodiments, the curable formulation may further comprise an additional curable material that features a plurality of photocurable groups. In some embodiments, a process of additive manufacturing a three-dimensional object may be provided. The process includes sequentially exposing, in a layerwise manner, a modeling material formulation to a curing condition suitable for hardening the curable formulation. This exposing is in a configured pattern corresponding to the shape of the object. In certain embodiments of the above process, the exposing is for a time period that ranges from 1 second to 120 seconds for each layer. In some embodiments, the curing condition in the process may comprise irradiation. In certain embodiments, the irradiation in the process may be at a wavelength within the UV-vis range. In some embodiments, the irradiation in the process may be at a wavelength that ranges from 300 to 800 nm, or from 300 to 600 nm, or from 300 to 500 nm, or from 350 to 450 nm. In certain embodiments of the above process, the irradiation is at a level that ranges from 1 to 150 mW/cm². In some embodiments, the additive manufacturing process may be implemented using DLP technology. In certain embodiments, a three-dimensional object obtained by the above process may be provided. In some embodiments, a three-dimensional object may be described, comprising, in at least a portion thereof, a hardened curable formulation as defined in any one of the above claims. In certain embodiments, an article-of-manufacturing may be provided, comprising the three-dimensional object of the above claim 33 or 34. In some embodiments, the curable formulation in any of the above embodiments may further comprise an aqueous carrier. In certain embodiments, the aqueous carrier in the curable formulation may be selected from a neutral aqueous solution or an acidic aqueous solution.

According to an aspect of some embodiments of the present invention, there is provided a three-dimensional object prepared by the method as described herein, which comprises a biological and/or a biocompatible material (e.g., hardened material) and minocycline or a compound featuring a tetracycline skeleton (a tetracycline compound). In some embodiments, the minocycline or the tetracycline compound is in an amount lower than its MEC or in an amount that does not provide an anti-bacterial effect or does not alter the anti-bacterial properties of the curable formulation used to form the object or of the object.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 presents comparative plots showing the storage modulus (G′) of formulations comprising methacrylated rh-Collagen (CMR; 5 mg/mL), LAP (0.5% by weight), and varying concentrations of minocycline.

FIG. 2 presents the absorbance spectrum of a of 10 mM HCl solutions containing 10 Mm (0.5% by weight) minocycline.

FIGS. 3A-C present comparative plots showing the UV absorbance spectra of 10 mM HCl solutions containing 10 mM minocycline (FIG. 3A), doxycycline (FIG. 3B), or tetracycline (FIG. 3C), compared to each of the tested tetracycline compounds respectively combined with 0.5% by weight NAP before and after irradiation at 385 nm.

FIGS. 4A-C present comparative plots showing the kinetics rate (time to reach storage modulus (G′)) of formulations comprising methacrylated rh-Collagen (CMR; 5 mg/mL), PEG-DA, NAP (0.5% by weight) and varying concentrations of minocycline (FIG. 4A), doxycycline (FIG. 4B) and tetracycline (FIG. 4C).

FIG. 5 presents a schematic illustration of an isometric view (left), the XY plane (middle) and the Z axis (right) of a calibration model used in printing resolution studies.

FIGS. 6A-C present pictorial representations of the XY plane (left photos) and Z axis (right photos) of a calibration model printed with curable formulations comprising methacrylated rh-Collagen (CMR; 5 mg/mL), PEG-DA, 0.5% by weight NAP and 0.2% by weight minocycline (FIG. 6A), doxycycline (FIG. 6B) and tetracycline (FIG. 6C).

### DESCRIPTION OF SPECIFIC EMBODIMENTS OF THE INVENTION

The present invention, in some embodiments thereof, relates to additive manufacturing, and more particularly, but not exclusively, to formulations usable in additive manufacturing of 3D objects featuring a biological material in at least a portion thereof and to additive manufacturing processes employing same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

As discussed hereinabove, currently practiced additive manufacturing methodologies oftentimes require the use of a photoabsorber (photoblocker) for achieving improved performance of both the process and the obtained three-dimensional object.

The present inventors have uncovered that minocycline can be advantageously used as a photoabsorber in such applications, and is particularly usable in 3D bioprinting of 3D objects that feature a biological material in at least a portion thereof. The inclusion of minocycline in curable formulations does not lead to precipitation of e.g., curable collagen and other biological components in the formulation, and does not substantially alter the viscosity of the formulation.

The present inventors have further uncovered that other members of the tetracycline family, which feature a tetracycline skeleton and are referred to herein also as tetracycline compounds or as compounds featuring a tetracycline skeleton, can be advantageously used as photoabsorbers in such applications.

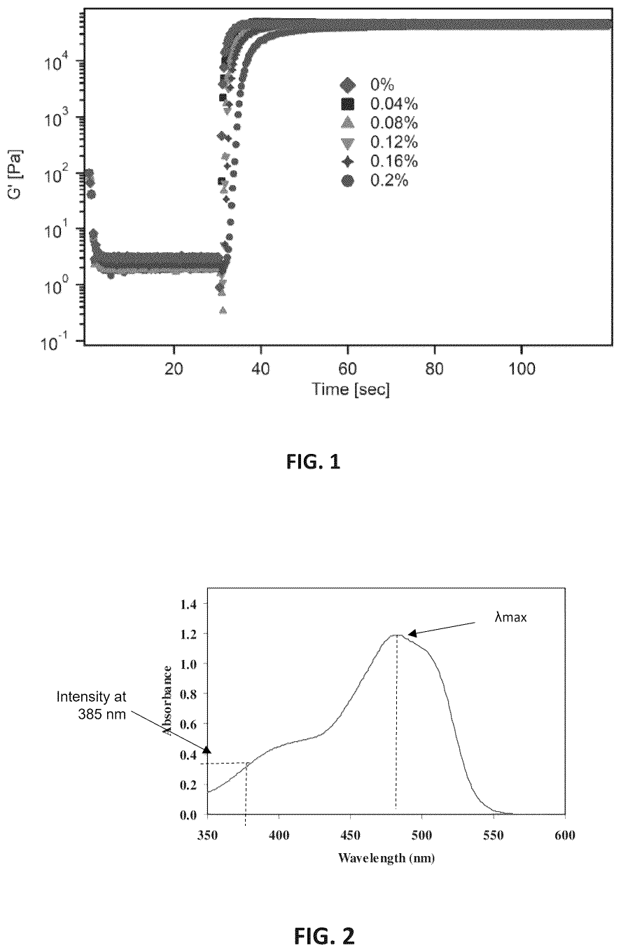

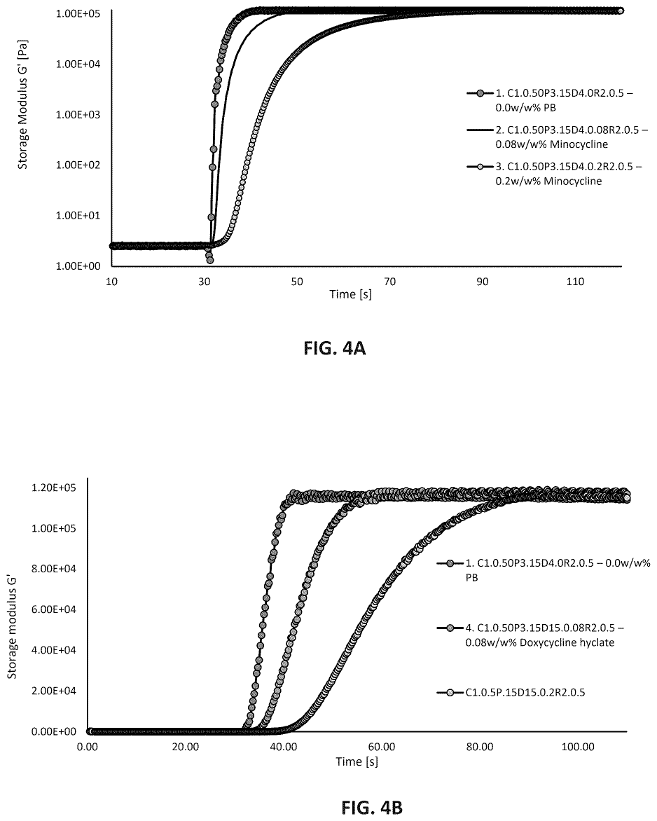

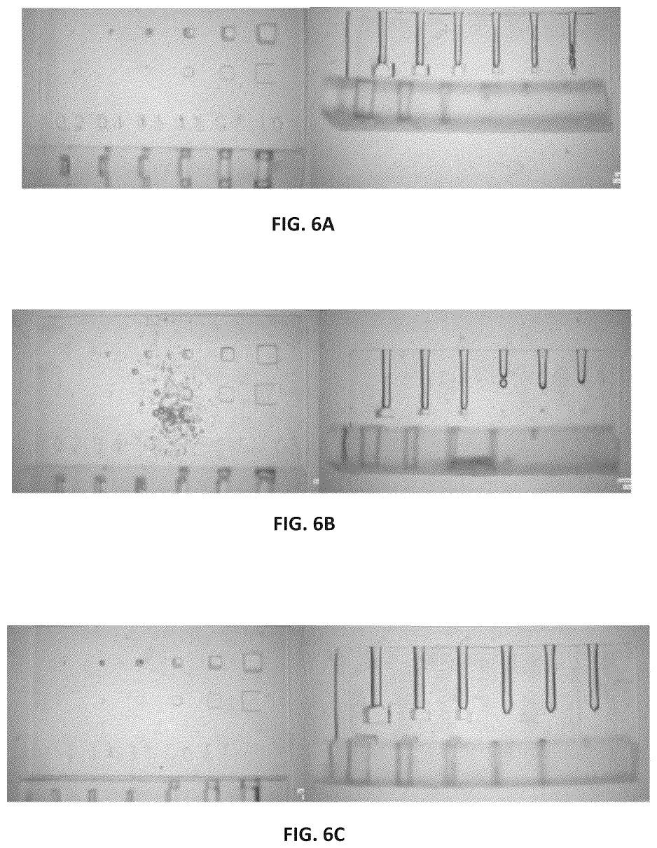

As described and demonstrated in the Examples section that follows, the present inventors have studied the compatibility and suitability of these compounds in terms of the process requirements, and have surprisingly uncovered that all of the exemplary compounds tested feature the required solubility in the curable formulation, the required UV absorbance profile which remains substantially unchanged in the presence of NAP also following irradiation at 385 nm (as shown in 3A-C), and, at the same time, do not adversely affect the solubility of other components in a curable formulation comprising the same, nor the hardening kinetics, the mechanical and rheological properties of the hardened material (as shown in FIGS. 1 and 4A-C), and the printing resolution (as shown in FIGS. 6A-C).

Embodiments of the present invention therefore relate to newly designed modeling material formulations (also referred to herein as “bioink compositions”), which comprise minocycline and/or other compounds featuring a tetracycline skeleton, as a photoabsorber, and to additive manufacturing processes utilizing same.

### Curable Formulation:

According to an aspect of some embodiments of the present invention, there is provided a curable formulation, which comprises a one or more photocurable materials, a photoinitiator, and minocycline. The minocycline is included in the formulation as a photo-absorber as described herein, and is therefore in an amount sufficient to absorb light at wavelength at which curing of the formulation is effected and to provide the desired effect of, for example, reducing the light penetration in the Z axis (through the printed layers) and/or increasing the required energy levels for the polymerization process in XY plane. Minocycline, also known as 7-Dimethylamino-6-demethyl-6-deoxytetracycline, is a semisynthetic tetracycline antibiotic medication used to treat a number of bacterial infections such as pneumonia. Minocycline has the following structure:

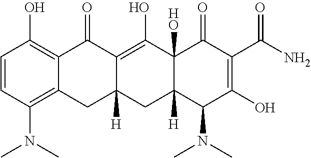

The term “minocycline” encompasses also salts, hydrates, solvates, enantiomers and diastereomers of minocycline.

Minocycline is a yellow crystalline powder that is sparingly soluble in water. According to some embodiments, the tetracycline is a minocycline. According to some embodiments, the minocycline is minocycline hydrochloride.

Minocycline hydrochloride has the following structure:

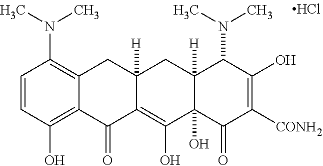 [SMILES: Cl ; CN(C)c1ccc(O)c2c1C[C@H]1C[C@H]3[C@H](N(C)C)C(O)=C(C(N)=O)C(=O)[C@@]3(O)C(O)=C1C2=O]

According to some embodiments of the present invention, the minocycline is included in the formulation in an amount that does not exhibit an antibacterial effect, or in an amount that is lower than an antibacterial effective amount (e.g., lower than its minimal effective concentration; MEC).

According to some of any of the embodiments described herein, an amount of the minocycline in the formulation is lower than 5%, preferably lower than 2%, preferably lower than 1%, preferably lower than 0.5%, and more preferably lower than 0.3%, by weight, of the total weight of the formulation.

According to some of any of the embodiments described herein, an amount of the minocycline in the formulation is lower than 5%, preferably lower than 2%, preferably lower than 1%, preferably lower than 0.5%, preferably lower than 0.3%, and more preferably lower than 0.2%, preferably lower than 0.1%, preferably lower than 0.09%, preferably lower than 0.08%, lower than 0.07%, lower than 0.06%, lower than 0.05%, lower than 0.04%, lower than 0.03% lower than 0.02%, lower than 0.01%, by weight, of the total weight of the formulation.

According to some of any of the embodiments described herein, an amount of the minocycline in the formulation ranges from about 0.01 to about 5%, preferably from about 0.01 to about 1%, more preferably from about 0.01 to about 0.5%, or from about 0.01 to about 0.3%, or from about 0.01 to about 0.2%, or from about 0.01 to about 0.1%, or from about 0.01 to about 0.15%, or from about 0.05 to about 0.5%, or from about 0.05 to about 0.15%, or from about 0.05 to about 0.2%, or from about 0.05 to about 0.1%, by weight of the total weight of the formulation, including any intermediate values and subranges therebetween.

According to an aspect of some embodiments of the present invention, there is provided a curable formulation, which comprises one or more photocurable materials, a photoinitiator, and a tetracycline compound, as defined herein. The tetracycline compound is included in the formulation as a photo-absorber as described herein, and is therefore in an amount sufficient to absorb light at wavelength at which curing of the formulation is effected and to provide the desired effect of, for example, reducing the light penetration in the Z axis (through the printed layers) and/or increasing the required energy levels for the polymerization process in XY plane.

A tetracycline compound, according to the present embodiments, is a compounds featuring a tetracyclic skeleton, naphthacene, typically a 6-deoxy-6-demethyltetracycline core structure.

This core structure can be modified with different functional groups and substitutions to create various tetracycline derivatives.

Common modifications include adding or substituting specific chemical groups on the tetracycline core structure, such as methyl groups, hydroxyl groups, or amino groups.

According to some of any of the embodiments described herein, compounds featuring a tetracycline skeleton can be collectively represented by Formula X:

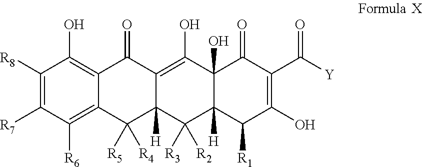

or a pharmaceutically acceptable salt thereof,
wherein:
Y is selected from amine, hydroxy, alkoxy, alkyl, O—NR′R″, thiol, thioalkoxy, thioaryloxy, aryl, cycloalkyl and halo;
R₁ is selected from hydrogen, amine, alkyl, cycloalkyl, aminoalkyl, aryl, hydroxy, alkoxy, aryloxy, thiol, thioalkoxy, thioaryloxy, halo, carboxy, amide, aminoether, carbamate, oxo, and like substituents (e.g., any of the substituents described herein);
R₂-R₅ are each independently selected from hydrogen, amine, alkyl, cycloalkyl, aminoalkyl, aminoether, aryl, hydroxy, alkoxy, aryloxy, thiol, thioalkoxy, thioaryloxy, halo, carboxy, amide, carbamate, oxo, and like substituents (e.g., any of the substituents described herein), or, alternatively, R₂ and R₃ and/or R₄ and R₅ form together an alkene, oxo or an alicyclic or heteroalicyclic ring (e.g., as defined herein); and
R₆-R₉ are each independently selected from hydrogen, amine, alkyl, cycloalkyl, aminoalkyl, aminoether, aryl, hydroxy, alkoxy, aryloxy, thiol, thioalkoxy, thioaryloxy, halo, carboxy, amide, carbamate, oxo, and like substituents (e.g., any of the substituents described herein), or, alternatively, R₆ and R₇ or R₇ and R₈ form together an alicyclic or heteroalicyclic ring (e.g., as defined herein).

It is to be noted that substituents not shown in Formula X are typically hydrogen, although in some embodiments, a hydrogen can be replaced by an alkyl (e.g., a lower alkyl as defined herein, or halo or hydroxy).

According to some of any of the embodiments described herein for Formula X, R₁ is amine. Preferably, the amine is a secondary amine or a tertiary amine, as defined herein, which is substituted by one or two substituents as defined herein, respectively. In exemplary embodiments, the amine is substituted by one or more alkyl substituents, preferably lower alkyl substituents (e.g., of from 1 to 6, or from 1 to 4, carbon atoms in length), which can be linear or branched, and which can be substituted or unsubstituted. In exemplary embodiments, the amine is a dimethylamine.

According to some of any of the embodiments described herein for Formula X, Y is amine, forming together with the adjacent carbonyl and amide substituent. In exemplary embodiments, the amine, or amide formed with the carbonyl, un unsubstituted.

According to some of any of the embodiments described herein for Formula X, R₂ and R₃ are each hydrogen.

According to some of any of the embodiments described herein for Formula X, Y is amine, R₁ is dimethylamine, and R₂ and R₃ are each hydrogen.

According to some of any of the embodiments described herein for Formula X, one or more of R₄-R₈ is halo, for example, chloro or fluoro.

According to some of any of the embodiments described herein for Formula X, of R₄ and R₅ form together an oxo or alkene substituent.

According to some of any of the embodiments described herein for Formula X, one or more of R₄-R₈ is hydroxy. In exemplary embodiments, one or more of R₄-R₆ is hydroxy.

Exemplary, non-limiting examples of compounds featuring a tetracycline skeleton include dihydrosteffimycin, demethyltetracycline, aclacinomycin, akrobomycin, baumycin, bromotetracycline, cetocyclin, chlortetracycline, clomocycline, daunorubicin, demeclocycline, doxorubicin, doxorubicin hydrochloride, doxycycline, lymecyclin, marcellomycin, meclocycline, meclocycline sulfosalicylate, methacycline, minocycline, minocycline hydrochloride, musettamycin, oxytetracycline, rhodirubin, rolitetracycline, rubomycin, serirubicin, steffimycin, tetracycline, and pharmaceutically acceptable salts of any one of the foregoing.

According to some of any of the embodiments described herein, compounds featuring a tetracycline skeleton include, without limitation, oxytetracycline, lymcycline, chlorotetracycline, oxytetarcycline, demeclocycline, meclocycline, methacycline, rolitetracycline, tigecycline, eravacycline, s arecycline, and omadacycline.

According to some of any of the embodiments described herein, the compound featuring a tetracycline skeleton is minocycline, as described herein in any of the respective embodiments, or a pharmaceutically acceptable salt thereof, as described herein (e.g., a hydrochloride salt). doxycycline, tetracycline and a pharmaceutically acceptable salt of any one of the foregoing.

According to some of any of the embodiments described herein, the compound featuring a tetracycline skeleton is tetracycline.

Tetracycline is a yellow crystalline powder that is soluble in water. According to some embodiments, the tetracycline is a tetracycline hydrochloride and has the following structure:

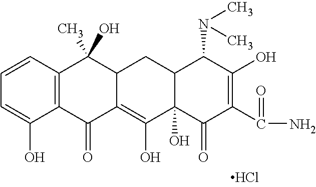 [SMILES: CN(C)[C@@H]1C(O)=C(C(N)=O)C(=O)[C@@]2(O)C(O)=C3C(=O)c4c(O)cccc4[C@@](C)(O)C3CC12 ; Cl]

According to some embodiments of the present invention, the tetracycline is included in the formulation is an amount that does not exhibit an antibacterial effect, or in an amount that is lower than an antibacterial effective amount (e.g., lower than its minimal effective concentration; MEC).

According to some of any of the embodiments described herein, an amount of the tetracycline in the formulation is lower than 5%, preferably lower than 2%, preferably lower than 1%, preferably lower than 0.5%, preferably lower than 0.3%, and more preferably lower than 0.2%, preferably lower than 0.1%, preferably lower than 0.09%, preferably lower than 0.08%, lower than 0.07%, lower than 0.06%, lower than 0.05%, lower than 0.04%, lower than 0.03% lower than 0.02%, lower than 0.01%, by weight, of the total weight of the formulation.

According to some of any of the embodiments described herein, an amount of the tetracycline by weight of the total weight of the formulation ranges from 1 to 10%, 1 to 5%, 0.01 to 5%, 0.01 to 4%, 0.01 to 3%, 0.01 to 2%, or from 0.01 to 1%, or from 0.01 to 0.5%, or from 0.01 to 0.3%, or from 0.01 to 0.2%, or from 0.01 to 0.1%, or from 0.01 to 0.15%, or from 0.05 to 0.5%, or from 0.05 to 0.15%, or from 0.05 to 0.2%, or from 0.05 to 0.1%, or from 0.08 to 0.2% by weight of the total weight of the formulation, including any intermediate values and subranges therebetween.

According to some of any of the embodiments described herein, the compound featuring a tetracycline skeleton is doxycycline.

Doxycycline, is a tetracycline compound derived from oxytetracycline, which features hydroxyl groups. It is typically available as doxycycline calcium, doxycycline hyclate and doxycycline monohydrate. Doxycycline hyclate and doxycycline monohydrate occur as yellow, crystalline powders. Doxycycline hyclate is soluble in water. doxycycline monohydrate is very slightly soluble in water. According to exemplary embodiments, the tetracycline compound is doxycycline hydrate.

Doxycycline hyclate has the following structure:

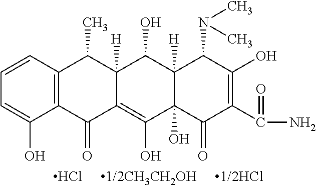

According to an aspect of some embodiments of the present invention, there is provided a curable formulation, which comprises one or more photocurable materials, a photoinitiator, and doxycycline. The doxycycline is included in the formulation as a photo-absorber as described herein, and is therefore in an amount sufficient to absorb light at wavelength at which curing of the formulation is effected and to provide the desired effect of, for example, reducing the light penetration in the Z axis (through the printed layers) and/or increasing the required energy levels for the polymerization process in XY plane.

According to some embodiments of the present invention, the doxycycline is included in the formulation is an amount that does not exhibit an antibacterial effect, or in an amount that is lower than an antibacterial effective amount (e.g., lower than its minimal effective concentration; MEC). According to some embodiments of the present invention, the doxycycline included in the formulation is an amount that does exhibit an antibacterial effect, or in an amount that is higher than an antibacterial effective amount (e.g., higher than its minimal effective concentration; MEC). According to some embodiments of the present invention three D models printable from such formulation can be advantageously used for pharmaceutical or therapeutic applications. According to some embodiments of the present invention three D models printable from such formulation can be used to prevent or treat antibacterial infection and inflammation.

According to some of any of the embodiments described herein, an amount of the doxycycline in the formulation is lower than 5%, preferably lower than 2%, preferably lower than 1%, preferably lower than 0.5%, preferably lower than 0.3%, and more preferably lower than 0.2%, preferably lower than 0.1%, preferably lower than 0.09%, preferably lower than 0.08%, lower than 0.07%, lower than 0.06%, lower than 0.05%, lower than 0.04%, lower than 0.03% lower than 0.02%, lower than 0.01%, by weight, of the total weight of the formulation.

According to some of any of the embodiments described herein, an amount of the doxycycline by weight of the total weight of the formulation ranges from 1 to 10%, or from 1 to 5%, or from 0.01 to 5%, or from 0.01 to 4%, or from 0.01 to 3%, or from 0.01 to 2%, or from 0.01 to 1%, or from 0.01 to 0.5%, or from 0.01 to 0.3%, or from 0.01 to 0.2%, or from 0.01 to 0.1%, or from 0.01 to 0.15%, or from 0.05 to 0.5%, or from 0.05 to 0.15%, or from 0.05 to 0.2%, or from 0.05 to 0.1%, or from 0.08 to 0.2% by weight of the total weight of the formulation, including any intermediate values and subranges therebetween.

According to some of any of the embodiments described herein, the curable formulation comprises one or more photocurable materials, and a photoinitiator.

The photoinitiator is selected in accordance with the curing mechanism (e.g., free-radical, cationic, etc.) and as suitable for the irradiation wavelength or wavelength range.

A free-radical photoinitiator may be any compound that produces a free radical on exposure to radiation such as ultraviolet or visible radiation and thereby initiates a polymerization reaction. Non-limiting examples of suitable photoinitiators include benzophenones (aromatic ketones) such as benzophenone, methyl benzophenone, Michler's ketone and xanthones; acylphosphine oxide type photo-initiators such as 2,4,6-trimethylbenzolydiphenyl phosphine oxide (TMPO) and salts thereof (e.g., Lithium phenyl-2,4,6-trimethylbenzoylphosphinate (LAP), Sodium phenyl-2,4,6-trimethylbenzoylphosphinate (NAP)) 2,4,6-trimethylbenzoylethoxyphenyl phosphine oxide (TEPO) and salts thereof, and bisacylphosphine oxides (BAPO's) and salts thereof); benzoins and bezoin alkyl ethers such as benzoin, benzoin methyl ether and benzoin isopropyl ether and the like. Examples of photoinitiators are alpha-amino ketone, and bisacylphosphine oxide (BAPO's).

Exemplary photoinitiators include, but are not limited to, those of the Irgacure® family, riboflavin, rose Bengal, and more.

A free-radical photo-initiator may be used alone or in combination with a co-initiator. Co-initiators are used with initiators that need a second molecule to produce a radical that is active in the photocurable free-radical systems. Benzophenone is an example of a photoinitiator that requires a second molecule, such as an amine, to produce a free radical. After absorbing radiation, benzophenone reacts with a ternary amine by hydrogen abstraction, to generate an alpha-amino radical which initiates polymerization of acrylates. Non-limiting examples of a class of co-initiators are alkanolamines such as triethylamine, methyldiethanolamine and triethanolamine.

Suitable cationic photoinitiators include, for example, compounds which form aprotic acids or Bronsted acids upon exposure to ultraviolet and/or visible light sufficient to initiate polymerization. The photoinitiator used may be a single compound, a mixture of two or more active compounds, or a combination of two or more different compounds, i.e., co-initiators. Non-limiting examples of suitable cationic photoinitiators include aryldiazonium salts, diaryliodonium salts, triarylsulphonium salts, triarylselenonium salts and the like. An exemplary cationic photoinitiator is a mixture of triarylsolfonium hexafluoroantimonate salts.

Non-limiting examples of suitable cationic photoinitiators include P-(octyloxyphenyl)phenyliodonium hexafluoroantimonate UVACURE 1600 from Cytec Company (USA), iodonium (4-methylphenyl)(4-(2-methylpropyl)phenyl)-hexafluorophosphate known as Irgacure 250 or Irgacure 270 available from Ciba Speciality Chemicals (Switzerland), mixed arylsulfonium hexafluoroantimonate salts known as UVI 6976 and 6992 available from Lambson Fine Chemicals (England), diaryliodonium hexafluoroantimonate known as PC 2506 available from Polyset Company (USA), (tolylcumyl) iodonium tetrakis(pentafluorophenyl) borate known as Rhodorsil® Photoinitiator 2074 available from Bluestar Silicones (USA), iodonium bis(4-dodecylphenyl)-(OC-6-11)-hexafluoro antimonate known as Tego PC 1466 from Evonik Industries AG (Germany).

According to some of any of the embodiments described herein, the photoinitiator is a free-radical photoinitiator, as described herein, for example, a photoinitiator of the acylphosphine oxide type, such as, for example, Lithium phenyl-2,4,6-trimethylbenzoylphosphinate (LAP) and/or Sodium phenyl-2,4,6-trimethylbenzoylphosphinate (NAP).

According to some of any of the embodiments described herein, the photoinitiator is an acyl phosphine oxide type photoinitiator such as a 2,4,6-trimethylbenzolydiphenyl phosphine oxide (TMPO) or a salt thereof (e.g., Lithium phenyl-2,4,6-trimethylbenzoylphosphinate (LAP), Sodium phenyl-2,4,6-trimethylbenzoylphosphinate (NAP), 2,4,6-trimethylbenzoylethoxyphenyl phosphine oxide (TEPO), and bisacylphosphine oxides (BAPO's).

According to some of any of the embodiments described herein, an amount of the photoinitiator in the formulation ranges from about 0.1 to about 10, or from about 0.1 to about 5, or from about 0.1 to about 3, or from about 0.1 to about 2, or from about 0.1 to about 1, % by weight, including any intermediate values and subranges therebetween.

The amount of the photoinitiator can be determined in accordance with additive manufacturing method, the amount of photoabsorber, light intensity, the layer thickness, the desired resolution and the viscosity suitable for the AM technique. A formulation according to the present embodiments can be prepared, for example, in accordance with the following:
Selecting an additive manufacturing technique;
Determining a viscosity suitable for the additive manufacturing technique;
Determining an amount of the photoinitiator that provides the desired viscosity; and
Preparing the formulation by mixing the determined amount of the photoinitiator with selected materials as described herein in any of the respective embodiments and any combination thereof, and minocycline or a tetracycline compound as described herein in any of the respective embodiments and any combination thereof, and optionally other components included in a curable formulation as described herein.

Herein throughout, and particularly in the context of additive manufacturing, the terms “method” and “process” are used interchangeably.

The above can be performed while using a look-up table which defines the viscosity that suitable for each of the varying AM techniques, and which defines the amount of the photoinitiator that provided the desired viscosity, or alternatively, which, based on the publicly available knowledge of viscosity values suitable for each AM technique.

Preparing the formulation can be performed manually or automatically, upon calculating, manually or automatically, the desired amount of the photoinitiator and/or photoabsorber (e.g., an amount that provides a desirable printing resolution).

As used herein, the term “curable” describes a formulation or a material that is capable of undergoing curing, or hardening (e.g., a change in viscosity or in storage modulus (G′)), as defined herein, when exposed to a suitable curing condition.

A curable formulation typically comprises one or more curable materials.

A curable material is typically hardened or cured by undergoing polymerization and/or cross-linking.

Curable materials are typically polymerizable materials, which undergo polymerization and/or cross-linking when exposed to a suitable curing condition or a suitable curing energy (a suitable energy source). Alternatively, curable materials are thermo-responsive materials, which solidify or harden upon exposure to a temperature change (e.g., heating or cooling). Optionally, curable materials are made of small particles (e.g., nanoparticles or nanoclays) which can undergo curing to form a hardened material. Further optionally, curable materials are biological materials which undergo a reaction to form a hardened or solid material upon a biological reaction (e.g., an enzymatically-catalyzed reaction).

In some of any of the embodiments described herein, a curable material is a photopolymerizable material, which polymerizes and/or undergoes cross-linking upon exposure to radiation, as described herein, and in some embodiments the curable material is a UV-curable material, which polymerizes or undergoes cross-linking upon exposure to UV-vis radiation, as described herein.

In some of any of the embodiments described herein, when a curable material is exposed to a curing condition (e.g., radiation), it polymerizes by any one, or combination, of chain elongation, entanglement and cross-linking. The cross-linking can be chemical and/or physical.

In some of any of the embodiments described herein, a curable material can be a mono-functional curable material or a multi-functional curable material.

Herein, a mono-functional curable material comprises one curable group—a functional group that can undergo polymerization, entanglement and/or cross-linking when exposed to a curing condition (e.g., radiation, presence of calcium ions).

A multi-functional curable material comprises two or more, e.g., 2, 3, 4 or more, curable groups. Multi-functional curable materials can be, for example, di-functional, tri-functional or tetra-functional curable materials, which comprise 2, 3 or 4 curable groups, respectively.

According to some of any of the embodiments described herein, the formulation comprises one or more photocurable materials, preferably UV-curable materials, which comprise one or more UV-curable groups.

In some of any of the embodiments described herein, at least a portion of the photocurable groups, are (meth)acrylic groups, such that the one or more of the photocurable materials include one or more acrylic materials.

Herein throughout, an acrylic material is used to collectively describe material featuring one or more acrylate, methacrylate, acrylamide and/or methacrylamide group(s).

Similarly, an acrylic group is used to collectively describe curable groups which are acrylate, methacrylate, acrylamide and/or methacrylamide group(s), collectively referred to herein also as (meth)acrylic groups.

Herein throughout, the term “(meth)acrylic” encompasses acrylic and methacrylic materials.

According to some of any of the embodiments described herein, the photocurable materials included in the formulation are biocompatible photocurable materials, which can be materials derived from a biological material and/or are biocompatible synthetic materials.

A formulation that comprises such photocurable materials is also referred to herein as a bioink formulation or bioink composition or simply as bioink.

According to some embodiments, the curable formulation is usable in additive manufacturing of a 3D object as described herein (e.g., in bioprinting). According to some embodiments, the composition is usable, or is for use, in the preparation of, or as, one or more modeling material formulation(s) for an additive manufacturing process (e.g., bioprinting). According to some embodiments, the additive manufacturing is of a three-dimensional object that comprises, in at least a portion thereof, a biological material, for example, a collagen material as described herein.

When the formulation comprises two or more photocurable materials, each material can feature one or more types of photocurable groups, and the photocurable groups in each material can be the same or different.

According to some of any of the embodiments described herein, the one or more biocompatible photocurable materials comprise a curable collagen.

The term “collagen” as used herein, refers to a polypeptide having a triple helix structure and containing a repeating Gly-X-Y triplet, where X and Y can be any amino acid but are frequently the amino acids proline and hydroxyproline. According to one embodiment, the collagen is a type I, II, III, V, XI, or biologically active fragments therefrom.

A collagen according to some of the present embodiments also refers to homologs (e.g., polypeptides which are at least 50%, at least 55%, at least 60%, at least 65%, at least 70%, at least 75%, at least 80%, at least 85%, at least 87%, at least 89%, at least 91%, at least 93%, at least 95% or more say 100% homologous to collagen sequences such as listed in Table A as determined using BlastP software of the National Center of Biotechnology Information (NCBI) using default parameters). The homolog may also refer to a deletion, insertion, or substitution variant, including an amino acid substitution, thereof and biologically active polypeptide fragments thereof.

According to a particular embodiment, the collagen is a human collagen.

In another embodiment, the collagen comprises a naturally occurring amino acid sequence of human collagen.

Table A below lists examples of collagen NCBI sequence numbers.

TABLE A

| Exemplary procollagen NCBI sequence number | SEQ ID NO: |
|---|---|
| P02452 | 1 |
| P08123 | 2 |

The annotation of SEQ ID NO: 1 is as follows:
Amino acids 1-22—signal peptide;
Amino acids 23-161—N-terminal peptide;
Amino acids 162-1218—collagen alpha-1(I) chain;
Amino acids 1219-1464—C-terminal peptide;

The annotation of SEQ ID NO: 2 is as follows:
Amino acids 1-22—signal peptide;
Amino acids 23-79—N-terminal peptide;
Amino acids 80-1119—collagen alpha-2(I) chain;
Amino acids 1120-1366—C-terminal peptide.

According to one embodiment, the collagen comprises a sufficient portion of its telopeptides such that under suitable conditions it is capable of forming fibrils.

Thus, for example, the collagen may be atelocollagen, a telocollagen or procollagen.

As used herein, the term “atelocollagen” refers to collagen molecules lacking both the N- and C-terminal propeptides typically comprised in procollagen and at least a portion of its telopeptides, but including a sufficient portion of its telopeptides such that under suitable conditions it is capable of forming fibrils.

The term “procollagen” as used herein, refers to a collagen molecule (e.g., human) that comprises either an N-terminal propeptide, a C-terminal propeptide or both. Exemplary human procollagen amino acid sequences are set forth by SEQ ID NOs: 3, 4, 5 and 6.

The term “telocollagen” as used herein, refers to collagen molecules that lack both the N- and C-terminal propeptides typically comprised in procollagen but still contain the telopeptides. The telopeptides of fibrillar collagen are the remnants of the N- and C-terminal propeptides following digestion with native N/C proteinases.

According to another embodiment, the collagen is devoid of its telopeptides and is not capable of undergoing fibrillogenesis.

According to another embodiment, the collagen is a mixture of the types of collagen above.

According to a particular embodiment, the collagen is genetically engineered using recombinant DNA technology (e.g., human collagen).

Methods of isolating collagen from animals are known in the art. Dispersal and solubilization of native animal collagen can be achieved using various proteolytic enzymes (such as porcine mucosal pepsin, bromelain, chymopapain, chymotrypsin, collagenase, ficin, papain, peptidase, proteinase A, proteinase K, trypsin, microbial proteases, and, similar enzymes or combinations of such enzymes) which disrupt the intermolecular bonds and remove the immunogenic non-helical telopeptides without affecting the basic, rigid triple-helical structure which imparts the desired characteristics of collagen (see U.S. Pat. Nos. 3,934,852; 3,121,049; 3,131,130; 3,314,861; 3,530,037; 3,949,073; 4,233,360 and 4,488,911 for general methods for preparing purified soluble collagen). The resulting soluble collagen can be subsequently purified by repeated precipitation at low pH and high ionic strength, followed by washing and re-solubilization at low pH.

Plants expressing collagen chains and procollagen are known in the art, see for example, WO 06/035442; Merle et al., FEBS Lett. 2002 Mar. 27; 515(1-3): 114-8. PMID: 11943205; and Ruggiero et al., 2000, FEBS Lett. 2000 Mar. 3; 469(1): 132-6. PMID: 10708770; and U.S. Patent Applications Publication Nos. 2002/098578 and 2002/0142391, as well as U.S. Pat. No. 6,617,431, each of which are incorporated herein by reference.

It will be appreciated that embodiments of the present invention also contemplate genetically modified forms of collagen/atelocollagen—for example collagenase-resistant collagens and the like [see, for example, Wu et al., Proc Natl. Acad Sci, Vol. 87, p.5888-5892, 1990].

Recombinant procollagen or telocollagen (e.g., human) may be expressed in any non-animal cell, including but not limited to plant cells and other eukaryotic cells such as yeast and fungus.

Plants in which procollagen or telocollagen may be produced (i.e., expressed) may be of lower (e.g., moss and algae) or higher (vascular) plant species, including tissues or isolated cells and extracts thereof (e.g., cell suspensions). Preferred plants are those which are capable of accumulating large amounts of collagen chains, collagen and/or the processing enzymes described herein below. Such plants may also be selected according to their resistance to stress conditions and the ease at which expressed components or assembled collagen can be extracted. Examples of plants in which human procollagen may be expressed include, but are not limited to tobacco, maize, alfalfa, rice, potato, soybean, tomato, wheat, barley, canola, carrot, lettuce and cotton.

Production of recombinant procollagen is typically effected by stable or transient transformation with an exogenous polynucleotide sequence encoding human procollagen.

Exemplary polynucleotide sequences encoding human procollagen are set forth by SEQ ID NOs: 7, 8, 9 and 10.

Production of human telocollagen is typically effected by stable or transient transformation with an exogenous polynucleotide sequence encoding human procollagen and at least one exogenous polynucleotide sequence encoding the relevant protease. Alternatively, a protease may be added following isolation of the recombinant procollagen.

The stability of the triple-helical structure of collagen requires the hydroxylation of prolines by the enzyme prolyl-4-hydroxylase (P4H) to form residues of hydroxyproline within the collagen chain. Although plants are capable of synthesizing hydroxyproline-containing proteins, the prolyl hydroxylase that is responsible for synthesis of hydroxyproline in plant cells exhibits relatively loose substrate sequence specificity as compared with mammalian P4H. Thus, production of collagen containing hydroxyproline only in the Y position of Gly-X-Y triplets requires co-expression of collagen and human or mammalian P4H genes [Olsen et al, Adv Drug Deliv Rev. 2003 Nov. 28; 55(12): 1547-67].

Thus, according to one embodiment, the procollagen or telocollagen is expressed in a subcellular compartment of a plant that is devoid of endogenous P4H activity.

As used herein, the phrase “subcellular compartment devoid of endogenous P4H activity” refers to any compartmentalized region of the cell which does not include plant P4H or an enzyme having plant-like P4H activity. According to one embodiment, the subcellular compartment is a vacuole, an apoplast or a chloroplast. According to a particular embodiment, the subcellular compartment is a vacuole.

According to another embodiment, the subcellular compartment is an apoplast.

Accumulation of the expressed procollagen in a subcellular compartment devoid of endogenous P4H activity can be effected via any one of several approaches.

For example, the expressed procollagen/telocollagen can include a signal sequence for targeting the expressed protein to a subcellular compartment such as the apoplast or an organelle (e.g., chloroplast).

Examples of suitable signal sequences include the chloroplast transit peptide (included in Swiss-Prot entry P07689, amino acids 1-57) and the Mitochondrion transit peptide (included in Swiss-Prot entry P46643, amino acids 1-28). Targeting to the vacuole may be achieved by fusing the polynucleotide sequence encoding the collagen to a vacuolar targeting sequence—for example using the vacuolar targeting sequence of the thiol protease aleurain precursor (NCBI accession P05167 GI: 113603):-MAHARVLLLALAVLATAAVAVASSSSFADSNPIRPVTDRAASTLA (SEQ ID NO: 14). Typically, the polynucleotide sequence encoding the collagen also comprises an ER targeting sequence. In one embodiment, the ER targeting sequence is native to the collagen sequence. In another embodiment, the native ER targeting sequence is removed and a non-native ER targeting sequence is added. The non-native ER targeting sequence may be comprised in the vacuolar targeting sequence. It will be appreciated, for it to traverse the ER and move on to the vacuole, the collagen sequence should be devoid of an ER retention sequence.

Alternatively, the sequence of the procollagen can be modified in a way which alters the cellular localization of the procollagen when expressed in plants.

The present invention contemplates genetically modified cells co-expressing both human procollagen and a P4H. In one embodiment, the P4H is capable of correctly hydroxylating the procollagen alpha chain(s) [i.e., hydroxylating only the proline (Y) position of the Gly-X-Y triplets]. P4H is an enzyme composed of two subunits, alpha and beta as set forth in Genbank Nos. P07237 and P13674. Both subunits are necessary to form an active enzyme, while the beta subunit also possesses a chaperon function.

The P4H expressed by the genetically modified cells of the present invention is preferably a human P4H. An exemplary polynucleotide sequence which encodes human P4H is SEQ ID Nos: 11 and 12. In addition, P4H mutants which exhibit enhanced substrate specificity, or P4H homologues can also be used. A suitable P4H homologue is exemplified by an Arabidopsis oxidoreductase identified by NCBI accession no: NP_179363.

Since it is essential that P4H co-accumulates with the expressed procollagen chain, the coding sequence thereof is preferably modified accordingly (e.g., by addition or deletion of signal sequences). Thus, the present invention contemplates using P4H polynucleotide sequences that are fused to vacuole targeting sequences. It will be appreciated that for targeting to the vacuole, when an endogenous ER retention sequence is present, it should be removed prior to expression.

In mammalian cells, collagen is also modified by Lysyl hydroxylase, galactosyltransferase and glucosyltransferase. These enzymes sequentially modify lysyl residues in specific positions to hydroxylysyl, galactosylhydroxylysyl and glucosylgalactosyl hydroxylysyl residues at specific positions. A single human enzyme, Lysyl hydroxylase 3 (LH3), as set forth in Genbank No. 060568, can catalyze all three consecutive modifying steps as seen in hydroxylysine-linked carbohydrate formation.

Thus, genetically modified cells according to some embodiments may also express mammalian LH3 (optionally fused to vacuole targeting sequences). It will be appreciated that for targeting to the vacuole, the endogenous ER retention sequence is removed prior to expression.

An LH3 encoding sequence such as that set forth by SEQ ID NO: 13, can be used for such purposes.

The procollagen(s) and modifying enzymes described above can be expressed from a stably integrated or a transiently expressed nucleic acid construct which includes polynucleotide sequences encoding the procollagen alpha chains and/or modifying enzymes (e.g. P4H and LH3) positioned under the transcriptional control of functional promoters. Such a nucleic acid construct (which is also termed herein as an expression construct) can be configured for expression throughout the whole organism (e.g. plant, defined tissues or defined cells), and/or at defined developmental stages of the organism. Such a construct may also include selection markers (e.g. antibiotic resistance), enhancer elements and an origin of replication for bacterial replication.

There are various methods for introducing nucleic acid constructs into both monocotyledonous and dicotyledenous plants (Potrykus, I., Annu. Rev. Plant. Physiol., Plant. Mol. Biol. (1991) 42:205-225; Shimamoto et al., Nature (1989) 338:274-276). Such methods rely on either stable integration of the nucleic acid construct or a portion thereof into the genome of the plant, or on transient expression of the nucleic acid construct, in which case these sequences are not inherited by the plant's progeny.

In addition, several methods exist in which a nucleic acid construct can be directly introduced into the DNA of a DNA-containing organelle such as a chloroplast.

There are two principle methods of effecting stable genomic integration of exogenous sequences, such as those included within the nucleic acid constructs of the present invention, into plant genomes:
(i) Agrobacterium-mediated gene transfer: Klee et al. (1987) Annu. Rev. Plant Physiol. 38:467-486; Klee and Rogers in Cell Culture and Somatic Cell Genetics of Plants, Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung, S. and Arntzen, C. J., Butterworth Publishers, Boston, Mass. (1989) p. 93-112.
(ii) Direct DNA uptake: Paszkowski et al., in Cell Culture and Somatic Cell Genetics of Plants, Vol. 6, Molecular Biology of Plant Nuclear Genes eds. Schell, J., and Vasil, L. K., Academic Publishers, San Diego, Calif. (1989) p. 52-68; including methods for direct uptake of DNA into protoplasts, Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; or by the direct incubation of DNA with germinating pollen, DeWet et al. in Experimental Manipulation of Ovule Tissue, eds. Chapman, G. P. and Mantell, S. H. and Daniels, W. Longman, London, (1985) p. 197-209; and Ohta, Proc. Natl. Acad. Sci. USA (1986) 83:715-719.

There are various methods of direct DNA transfer into plant cells. In electroporation, protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals, tungsten particles or gold particles, and the microprojectiles are physically accelerated into cells or plant tissues.

Regardless of the transformation technique employed, once collagen-expressing progeny are identified, such plants are further cultivated under conditions which maximize expression thereof. Progeny resulting from transformed plants can be selected, by verifying presence of exogenous mRNA and/or polypeptides by using nucleic acid or protein probes (e.g. antibodies). The latter approach enables localization of the expressed polypeptide components (by for example, probing fractionated plants extracts) and thus also verifies the plant's potential for correct processing and assembly of the foreign protein.

Following cultivation of such plants, the telopeptide-comprising collagen is typically harvested. Plant tissues/cells are preferably harvested at maturity, and the procollagen molecules are isolated using extraction approaches. Preferably, the harvesting is effected such that the procollagen remains in a state that it can be cleaved by protease enzymes. According to one embodiment, a crude extract is generated from the transgenic plants of the present invention and subsequently contacted with the protease enzymes.

As mentioned, the propeptide or telopeptide-comprising collagen may be incubated with a protease to generate atelocollagen or collagen prior to solubilization. It will be appreciated that the propeptide or telopeptide-comprising collagen may be purified from the genetically engineered cells prior to incubation with protease, or alternatively may be purified following incubation with the protease. Still alternatively, the propeptide or telopeptide-comprising collagen may be partially purified prior to protease treatment and then fully purified following protease treatment. Yet alternatively, the propeptide or telopeptide-comprising collagen may be treated with protease concomitant with other extraction/purification procedures.

Exemplary methods of purifying or semi-purifying the telopeptide-comprising collagen of the present invention include, but are not limited to salting out with ammonium sulfate or the like and/or removal of small molecules by ultrafiltration.

The protease used for cleaving the recombinant propeptide or telopeptide comprising collagen is not necessarily derived from an animal. Exemplary proteases include, but are not limited to certain plant derived proteases e.g., ficin (EC 3.4.22.3) and certain bacterial derived proteases e.g., subtilisin (EC 3.4.21.62), neutrase. The present inventors also contemplate the use of recombinant enzymes such as rhTrypsin and rhPepsin. Several such enzymes are commercially available e.g. Ficin from Fig tree latex (Sigma, catalog #F4125 and Europe Biochem), Subtilisin from Bacillus licheniformis (Sigma, catalog #P5459) Neutrase from bacterium Bacillus amyloliquefaciens (Novozymes, catalog #PW201041) and TrypZean™, a recombinant human trypsin expressed in corn (Sigma catalog #T3449).

In some of any of the embodiments described herein, the recombinant human collagen is a recombinant human type I collagen.

In some of any of the embodiments described herein, the recombinant human collagen is a plant-derived recombinant human collagen and in some embodiments the plant is tobacco. An exemplary collagen is described in Stein H. (2009) Biomacromolecules; 10:2640-5, WO 2006/035442, WO 2009/053985, WO 2011/064773, WO 2013/093921, and WO 2014/147622.

In some of any of the embodiments described herein, the recombinant human collagen is a recombinant human type I collagen comprising two al units having the amino acid sequence which is at least 90% homologous, at least 91% homologous, 92% homologous, at least 93% homologous, at least 94% homologous, at least 95% homologous, at least 96% homologous, at least 97% homologous, at least 98% homologous, at least 99% homologous or 100% homologous to the sequence as set forth in SEQ ID NO:15 as determined using BlastP software of the National Center of Biotechnology Information (NCBI) using default parameters), and one α2 unit having the amino acid sequence which is at least 90% homologous, at least 91% homologous, 92% homologous, at least 93% homologous, at least 94% homologous, at least 95% homologous, at least 96% homologous, at least 97% homologous, at least 98% homologous, at least 99% homologous or 100% homologous to the sequence as set forth in SEQ ID NO:6. According to a particular embodiment, the type I collagen consists of two al units which consists of the sequence as set forth in SEQ ID NO: 15 and one α2 unit consisting of the sequence as set forth in SEQ ID NO:6, as determined using BlastP software of the National Center of Biotechnology Information (NCBI) using default parameters).

In some of any of the embodiments described herein, the al unit is encoded by a polynucleotide sequence being at least which is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%, e.g., 100% identical to the nucleic acid sequence as set forth in SEQ ID NO: 16. The α2 unit is encoded by a polynucleotide sequence being at least which is at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 93%, at least about 94%, at least about 95%, at least about 96%, at least about 97%, at least about 98%, at least about 99%, e.g., 100% identical to the nucleic acid sequence as set forth in SEQ ID NO: 10.

Identity (e.g., percent homology) can be determined using any homology comparison software, including for example, the BlastN software of the National Center of Biotechnology Information (NCBI) such as by using default parameters.

In some of any of the embodiments described herein, the human recombinant collagen (rhCollagen) as described herein in any of the respective embodiments is a monomeric rhCollagen.

By “monomeric” it is meant a rhCollagen as described herein which is soluble in an aqueous solution and does not form fibrillar aggregates.

In some of any of the embodiments described herein, the human recombinant collagen (rhCollagen) as described herein in any of the respective embodiments is a fibrillar rhCollagen.

By “fibrillar” it is meant a rhCollagen as described herein which is in a form of fibrillar aggregates in an aqueous solution containing same. Typically, but not obligatory, fibrillar rhCollagen is formed by subjecting monomeric rhCollagen to a fibrillogenesis buffer, typically featuring a basic pH. An exemplary procedure for forming fibrillar rhCollagen, is described in WO 2018/225076.

According to some of any of the embodiments described herein, the collagen is a tissue-derived collagen, for example, a tissue-derived human Type I collagen.

By “curable collagen” it is meant a collagen as described herein in any of the respective embodiments (e.g., human recombinant collagen), which features one or more curable groups as defined herein. According to some of any of the embodiments described herein, the curable collagen is a multi-functional curable material that comprises a plurality of curable groups, as defined herein.

The terms “curable collagen” and “collagen featuring one or more (or at least one) curable groups” are used herein interchangeably.

According to some of any of the embodiments described herein, the curable collagen comprises an amino acid sequence as described herein in any of the respective embodiments, and features one or more, preferably a plurality of, curable groups generated at least a portion of the amino acid residues forming the collagen, preferably by covalent attachment of a compound that comprises a curable group to functional groups of the side chains of the amino acid residues. Alternatively, or in addition, curable groups can be generated at the N-terminus and/or C-terminus of one or more the units forming the collagen, for example, by covalent attachment of a compound that comprises a curable group to a respective amine or carboxylate.

According to some of any of the embodiments described herein, the curable collagen is as described in WO 2018/225076.

According to some of any of the embodiments described herein, a curable collagen describes a collagen as described herein (e.g., rhCollagen as described herein in any of the respective embodiments) to which one or more curable groups are attached directly (e.g., by means of a covalent bond to a respective lysine residue of the collagen), or are not attached by means of an elastic moiety that terminates by a curable group as described herein.

According to some of any of the embodiments described herein, at least a portion of the curable groups in a curable collagen as described herein are cross-linkable photocurable groups, which undergo cross-linking when exposed to irradiation as the curing condition.

In some embodiments, curable groups can undergo polymerization and/or cross-linking via free-radical mechanism.

Exemplary such curable groups include acrylic groups, including acrylate, methacrylate, acrylamide and methacrylamide groups, which are collectively referred to herein as (meth)acrylic groups. Other free-radical curable groups may include thiols, vinyl ethers and other groups that feature a reactive double bond.

In some embodiments, curable groups can undergo polymerization and/or cross-linking via other mechanisms, such as cationic polymerization, or (cationic or anionic) ring opening polymerization. Exemplary such curable groups include, but are not limited to, epoxy-containing groups, caprolactam, caprolactone, oxetane, and vinyl ether.

Other curable groups can include, for example, formation of amide bonds between functional carboxylate and amine group (each being a curable group that reacts with the other and can effect cross-linking); formation of an imine bond between and amine and an aldehyde group; formation of urethane between isocyanate groups and hydroxyl groups via polycondensation in the presence of a catalyst and/or upon exposure to UV radiation; and formation of disulfide bonds between two thiols.

Any other photocurable groups are contemplated.

The photocurable groups in the curable collagen can be generated by means of chemical reactions between a material that comprises or can generate the photocurable group(s) when reacted with chemically-compatible functional groups present in the collagen, as described herein, either directly, or be means of a spacer or a linker, using chemistries well known in the art. For example, a material that comprises a curable group and a functional group can be reacted with a compatible functional group in the collagen, for example, a functional group in an amino acid side chain, such that the curable group is a substituent of the amino acid side chain.

In some embodiments, a compatible functional group is first generated within the collagen by chemical modification of chemical groups of the collagen, and is then reacted with a material that comprises or generates a curable group upon the reaction.

Whenever a curable collagen comprises more than one photocurable groups, the photocurable groups can be the same or different.

According to some of any of the embodiments described herein, at least a portion, or all, of the curable groups in a curable collagen of the present embodiments are photopolymerizable groups (e.g., UV-curable groups) that are capable of undergoing polymerization and/or cross-linking upon exposure to irradiation as described herein.

According to some of any of the embodiments described herein the curable group is a photocurable or photopolymerizable group (e.g., a (meth)acrylic group such as an acrylate or methacrylate).

Alternatively, one or more of the curable groups is a thiol-containing group, which provides disulfide bridge upon curing.

Alternatively, one or more of the curable groups is cured upon undergoing a chemical reaction, such as glycation or conjugation (using coupling agents such as EDC).

According to some embodiments, one or more of the curable groups comprise an amine and a carboxyl group which form peptide bonds upon curing.

According to some of any of the embodiments described herein, at least a portion, or all, of the curable groups in a curable collagen of the present embodiments are (meth)acrylic groups, as defined herein.

According to some of any of the embodiments described herein, an acrylic group such as methacrylamide can be generated by reacting an acrylate or methacrylate (e.g., acrylic acid, methacrylic acid, acrylic or methacrylic ester, acrylic or methacrylic anhydride) with an amine functional group (of, for example, lysine residues).

According to some of any of the embodiments of the present invention, the number of the curable groups in a curable collagen as described herein can determine the degree of curing (e.g., the degree of cross-linking) and can be manipulated in order to achieve a desired curing (e.g., cross-linking) degree.

According to some of any of the embodiments described herein, the curable collagen features a plurality of acrylamide or methacrylamide curable groups generated by reacting with lysine residues as described herein.

According to some of any of the embodiments described herein, the curable collagen features a plurality of acrylamide or methacrylamide curable groups substituting the amine groups of lysine residues in the collagen.

In some embodiments, at least 20%, or at least 30%, or at least 40%, or at least 50%, or at least 60%, or at least 70%, of the lysine residues in the collagen are substituted by a methacrylamide or acrylamide group. In some embodiments, the curable collagen features from 10% to 90%, or from 10% to 80%, or from 10% to 60%, or from 10 to 50%, or from 20 to 90%, or from 20 to 80%, or from 20 to 60%, or from 20 to 50%, of its lysine residues substituted by a methacrylamide or acrylamide group, including any intermediate values and subranges therebetween.

A curable collagen (e.g., rhCollagen) as described herein can be prepared by reacting a material that comprises a curable group or which generates a curable group with the collagen (e.g., rhCollagen), as described, for example, in WO 2018/225076.

The number of curable groups in the collagen (e.g., rhCollagen) can be controlled by manipulating the amount of the material reacted with the collagen (e.g., rhCollagen) for generating the curable groups.

According to some of any of the embodiments described herein, the curable collagen is a recombinant human type I collagen as described herein in any of the respective embodiments and any combination thereof.

According to some of any of the embodiments described herein, the one or more of the curable materials comprise(s) collagen, as described herein in any of the respective embodiments and any combination thereof, including a curable collagen as described herein, which features a plurality of curable elastic moieties covalently attached to the collagen.

Such a curable material can be regarded as curable collagen, which, upon exposure to a curing condition, undergoes hardening, e.g., via polymerization and/or cross-linking, as described herein, by means of polymerization and/or cross-linking of at least the curable elastic moieties, and optionally also other curable groups on the collagen (in case a curable collagen is conjugated with the plurality of curable elastic moieties).

By “plurality” it is meant two or more, preferably three or more, moieties that are attached to the collagen.

The terms “elastic” and “elastomeric” as indicated herein with regard to a group (e.g., curable group) or material (e.g., curable material) are used herein interchangeably.

The plurality of elastomeric moieties can be the same or different. When different, the difference can be in the chemical composition or stereochemistry of the elastic moiety and/or in the type of the curable group and/or in the position of the curable group.

An elastic moiety that features a curable group is also referred to herein interchangeably as a “curable elastic moiety” or as a “curable elastomeric moiety” or as “an elastomeric moiety that features a curable group”, or as “an elastic moiety that features a curable group”, and all means that an elastic or elastomeric moiety features one or more curable groups.

According to some of any of the embodiments described herein, the elastomeric moiety is a moiety that confers elasticity to the hardened material formed upon polymerization and/or cross-linking of the respective curable material. Such moieties typically comprise alkyl, alkylene chains, hydrocarbon chains, alkylene glycol groups or chains (e.g., oligo or poly(alkylene glycol) as defined herein, urethane, oligourethane or polyurethane moieties, as defined herein, and the like, including any combination (e.g., co-polymers) of the foregoing.

By “elasticity” it is meant an ability of a deformed material body to return to its original shape and size when the forces causing the deformation are removed. Elasticity can be determined, for example, by the determining storage modulus, elastic modulus, and/or shear recovery rate of the hardened material. Exemplary methods of determining these parameters are described in the Examples section that follows. Other methods are well-known in the art and are also contemplated.

A curable elastomeric moiety can be a mono-functional elastomeric moiety, that comprises one curable group, or a multi-functional curable moiety, that comprises two or more curable groups.

A mono-functional curable elastomeric moiety according to some embodiments of the present invention can be derived from a vinyl-containing compound represented by Formula I:

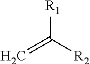

wherein at least one of R₁ and R₂ is and/or comprises an elastomeric moiety, as described herein.

The (═CH₂) group in Formula I represents a polymerizable group, and is, according to some embodiments, a UV-curable group, such that the elastomeric curable material and the moiety derived therefrom is a UV-curable material or moiety.

For example, R₁ is or comprises an elastomeric moiety as defined herein and R₂ is, for example, hydrogen, C(1-4)alkyl, C(1-4)alkoxy, or any other substituent, and is preferably hydrogen or alkyl such as methyl.

In some embodiments, R₁ is a carboxylate, and the curable elastomeric moiety is a mono-functional (meth)acrylate. In some of these embodiments, R₂ is hydrogen, and the curable elastomeric moiety is mono-functional acrylate. In some of these embodiments, R₂ is methyl, and the curable elastomeric moiety is a mono-functional methacrylate. Curable moieties in which R₁ is carboxylate and R₂ is hydrogen or methyl are collectively referred to herein as “(meth)acrylates”.

In some of any of these embodiments, the carboxylate group, —C(═O)—ORa, comprises Ra which is or comprises an elastomeric moiety as described herein, and which is linked to the collagen as described herein. In some embodiments, the Ra elastomeric moiety terminates by a reactive group that is used for conjugating a compound of Formula I to a respective group of the collagen (e.g., a carboxylate group that reacts with amine groups of lysine residues of the collagen). In some embodiments, R₁ is amide, and the elastomeric moiety is a mono-functional acrylamide. In some of these embodiments, R₂ is hydrogen, and the curable elastomeric moiety is a mono-functional acrylamide. In some of these embodiments, R₂ is methyl, and the curable elastomeric moiety is a mono-functional methacrylamide. Curable elastomeric moieties in which R₁ is amide and R₂ is hydrogen or methyl are collectively referred to herein as “(meth)acrylamide”.

(Meth)acrylates and (meth)acrylamides are collectively referred to herein as (meth)acrylic materials.

In multi-functional elastomeric moieties, the two or more polymerizable groups are linked to one another via an elastomeric moiety, as described herein, and the elastomeric moiety is also linked to the collagen.

In some embodiments, a multi-functional elastomeric moiety can be represented by Formula I as described herein, in which R₁ comprises an elastomeric material that terminates by a polymerizable group, as described herein.

For example, a di-functional elastomeric curable moiety can be represented by Formula I*:

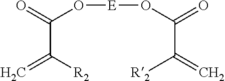

wherein E is an elastomeric linking moiety as described herein, and R′₂ is as defined herein for R₂.

In some embodiments, a multi-functional (e.g., di-functional, tri-functional or higher) elastomeric curable material can be collectively represented by Formula II:

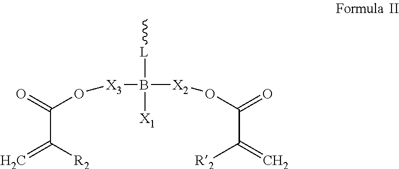

Wherein:
L represents an attachment point to the collagen, and can be a bond, or a linking moiety such as an alkylene, or a hydrocarbon chain;
R₂ and R′₂ are as defined herein;
B is a tri-functional or tetra-functional branching unit as defined herein (depending on the nature of X₁);
X₂ and X³ are each independently absent, an elastomeric moiety as described herein, or is selected from an alkyl, a hydrocarbon, an alkylene chain, a cycloalkyl, an aryl, an alkylene glycol, a urethane moiety, and any combination thereof; and
X₁ is absent or is selected from an alkyl, a hydrocarbon, an alkylene chain, a cycloalkyl, an aryl, an alkylene glycol, a urethane moiety, and an elastomeric moiety, each being optionally being substituted (e.g., terminated) by a meth(acrylate) moiety (O—C(═O)CR″₂═CH₂), and any combination thereof, or, alternatively, X₁ is:

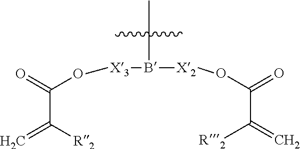

wherein:
the curved line represents the attachment point;
B′ is a branching unit, being the same as, or different from, B;
X′₂ and X′₃ are each independently as defined herein for X₂ and X₃; and
R″₂ and R″₂ are as defined herein for R₂ and R′₂.
provided that at least one of X₁, X₂ and X₃ is or comprises an elastomeric moiety as described herein.

The term “branching unit” as used herein describes a multi-radical, preferably aliphatic or alicyclic group. By “multi-radical” it is meant that the linking moiety has two or more attachment points such that it links between two or more atoms and/or groups or moieties.

That is, the branching unit is a chemical moiety that, when attached to a single position, group or atom of a substance, creates two or more functional groups that are linked to this single position, group or atom, and thus “branches” a single functionality into two or more functionalities.

In some embodiments, the branching unit is derived from a chemical moiety that has two, three or more functional groups. In some embodiments, the branching unit is a branched alkyl or a branched linking moiety as described herein.

Multi-functional elastomeric curable materials featuring 4 or more polymerizable groups are also contemplated, and can feature structures similar to those presented in Formula II, while including, for example, a branching unit B with higher branching, or including an X₁ moiety featuring two (meth)acrylate moieties as defined herein.

In some embodiments, the elastomeric moiety, e.g., Ra in Formula I or the moiety denoted as E in Formulae I* and II, is or comprises an alkyl, which can be linear or branched, and which is preferably of 3 or more or of 4 or more carbon atoms; an alkylene chain, preferably of 3 or more or of 4 or more carbon atoms in length; an alkylene glycol as defined herein, an oligo (alkylene glycol), or a poly(alkylene glycol), as defined herein, preferably of 4 or more atoms in length, a urethane, an oligourethane, or a polyurethane, as defined herein, preferably of 4 or more carbon atoms in length, and any combination of the foregoing.

In some of any of the embodiments described herein, the elastomeric curable moiety is a (meth)acrylic curable moiety, as described herein, and in some embodiments, it is an acrylate.

In some of any of the embodiments described herein, the elastomeric curable moiety is a mono-functional elastomeric curable moiety, and is some embodiments, the mono-functional elastomeric curable material is represented by Formula I, wherein R₁ is —C(═O)—ORa or —C(═O)—NH—Ra and Ra is or comprises a poly(alkylene glycol) chain (e.g., of 4 or more, preferably 6 or more, preferably 8 or more, alkylene glycol groups), as defined herein.

In some of any of the embodiments described herein, the elastomeric curable moiety is a mono-functional elastomeric curable moiety, and is some embodiments, the mono-functional elastomeric curable material is represented by Formula I, wherein R₁ is —C(═O)—NH—Ra and Ra is or comprises a poly(alkylene glycol) chain (e.g., of 4 or more, preferably 6 or more, preferably 8 or more, alkylene glycol groups), as defined herein.

In some of any of the embodiments described herein, the elastomeric curable moiety is selected such that an elastomeric material from which it is derived provides when hardened (alone), a polymeric material that features a Tg lower than 0 °C or lower than −10 °C.

According to some of any of the embodiments described herein, in each of the curable elastomeric moieties, the curable group is at a terminus of each of the elastic moiety, such that, for example, in Formula I, when Ra is or comprises an elastomeric moiety, the latter is attached at its other end to the collagen. It should be noted that curable elastomeric moieties that feature, alternatively or in addition, curable groups at a position other than the terminus are also contemplated.

According to the present embodiments, in at least a portion, or each, of the curable elastomeric moieties, the curable group is a photocurable or photopolymerizable group, for example, a UV-curable group.

According to some of any of the embodiments described herein, in at least a portion, or each, of the curable elastomeric moieties, the curable group is a (meth)acrylic group. In some of these embodiments, the curable group is a (meth)acrylamide, and in some embodiments, a methacrylamide.

According to some of any of the embodiments described herein, in at least a portion, or each, of the curable elastomeric moieties, the elastic moiety is or comprises a poly(alkylene glycol) moiety, as described herein, and in some of these embodiments, the poly(alkylene glycol) moiety features a (meth)acrylic group (e.g., a (meth)acrylamide) at its terminus.

In at least a portion, or each, of the elastomeric moieties, the curable group is linked to the elastomeric (e.g., poly(alkylene glycol)) moiety via a linking moiety, such that, for example, a (meth)acrylic curable elastomeric moiety is represented by Formula A:

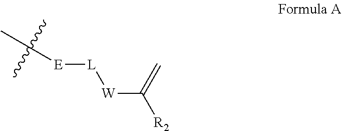

wherein:
R₂ is as defined herein in any of the respective embodiments,
W is —(C═X)—O— or —C═X—NRa;
X is O or S;
Ra is hydrogen or alkyl;
L is a linking moiety; and
E is the elastomeric moiety, for example, a poly(alkylene glycol) as defined herein in any of the respective embodiments.

The dashed line represents an attachment point to the collagen (e.g., via a covalent bond as described herein).

In some embodiments, the linking moiety is or comprises an alkylene chain, preferably a short alkylene chain of no more than 10, or no more than 8, or no more than 6, or no more than 4, carbon atoms in length, for example, of from 1 to 6, or from 1 to 4, carbon atoms in length.

In some embodiments, the linking moiety is attached to the elastomeric moiety (e.g., poly(alkylene glycol) moiety via a bond such as an amide bond, a carbamate bond, an ether bond, an ester bond, a thioester bond, a thioamide bond, a thiocarbamate bond, a sulfonamide bond, and the like. In some of these embodiments, the linking moiety is connected to the elastomeric moiety via a carbamate bond.

According to some of any of the embodiments described herein, when the elastomeric moiety is or comprises a poly(alkylene glycol) moiety, an average molecular weight of the plurality of such moieties is at least 1000 grams/mol, or at least 2000 grams/mol, or at least 3000 grams/mol or at least 4000 grams/mol, for example, in a range of from about 1000 to about 20000 grams/mol, from about 2000 to about 20000 grams/mol, from about 3000 to about 20000 grams/mol, from about 4000 to about 20000 grams/mol, from about 1000 to about 15000 grams/mol, from about 2000 to about 15000 grams/mol, from about 3000 to about 15000 grams/mol, or from about 4000 to about 15000 grams/mol, from about 1000 to about 10000 grams/mol, from about 2000 to about 10000 grams/mol, from about 3000 to about 10000 grams/mol or from about 4000 to about 10000 grams/mol, or from about 1000 to about 8000 grams/mol, from about 2000 to about 8000 grams/mol, from about 3000 to about 8000 grams/mol, from about 4000 to about 8000 grams/mol, including any intermediate values and subranges therebetween.

Each of the curable elastomeric moieties as described herein in any of the respective embodiments can be linked to the collagen by means of a covalent bond between a functional (reactive) group of the curable elastomeric moiety and a functional group of the collagen, preferably, a functional group at a collagen terminus and/or a functional group of an amino acid side chain. The curable elastomeric moieties can be linked to the collagen via the same of different bonds.

According to some of any of the embodiments described herein, at least a portion, or all, of the elastomeric moieties are covalently attached to lysine residues of the collagen.

According to some of any of the embodiments described herein, at least a portion, or all, of the elastomeric moieties are covalently attached to the collagen via a carbamate bond.

According to some of any of the embodiments described herein, at least a portion, or all, of the elastomeric moieties are covalently attached to lysine residues of the collagen via a bond such as an amide bond, a carbamate bond, a thioamide bond, a thiocarbamate bond, a sulfonamide bond, a hydrazine bond, a hydrazine bond, and the like.

According to some of any of the embodiments described herein, at least a portion, or all, of the elastomeric moieties are covalently attached to lysine residues of the collagen via a carbamate bond.

According to some of any of the embodiments described herein, at least 1%, for example, from 1 to 20%, or from 1 to 10%, or at least 2%, for example, from 2 to 20%, or from 2 to 10%, of the lysine residues in the collagen have the curable elastomeric moieties covalently attached thereto (e.g., via a carbamate bond).

According to some of any of the embodiments described herein, the collagen to which the curable elastomeric moieties are attached is as described herein in any of the respective embodiments.

According to some of any of the embodiments described herein, the collagen is a human Type I collagen, as described herein.

According to some of any of the embodiments described herein, the collagen is a recombinant collagen, as described herein.

According to some of any of the embodiments described herein, the collagen is a plant-derived recombinant collagen, as described herein.

According to some of any of the embodiments described herein, the collagen is a plant-derived recombinant human Type I collagen, as described herein, for example, tobacco-derived collagen.

According to some of any of the embodiments described herein, the collagen to which the elastomeric curable groups are attached features a plurality of curable groups, e.g., photocurable groups, other than the curable elastomeric moieties, such that in some embodiments, the conjugate comprises a curable collagen as described herein in any of the respective embodiments, to which are attached curable elastomeric moieties as described herein in any of the respective embodiments and any combination thereof.

According to some of any of the embodiments described herein, the curable collagen is an elastomeric curable collagen, which is a conjugate of a curable collagen as described herein in any of the respective embodiments and any combination thereof and a plurality of elastic moieties attached to the curable collagen. In some of these embodiments, the elastic moieties do not feature a curable group. The elastic moieties can be the same or different and each can independently an elastic moiety as described herein in any of the respective embodiments.

In exemplary embodiments, at least a portion of the elastic moieties comprise a poly(alkylene glycol) moiety as described herein in any of the respective embodiments and any combination thereof. In some of these exemplary embodiments, at least a portion of the poly(alkylene glycol) moieties are “capped”, namely, terminate with a group other than hydroxy, for example, by an alkoxy (e.g., methoxy). In exemplary embodiments, the conjugate comprises a plurality poly(ethylene glycol) moieties, each having an average molecular weight of about 5,000 grams/mol, and each being terminated by a methoxy group.

According to some of any of the embodiments described herein, the curable formulation provides, when hardened, a hydrogel material, formed upon cross-linking the photocurable materials within an aqueous carrier such as described herein.

Herein and in the art, the term “hydrogel” describes a three-dimensional fibrous network containing at least 20%, typically at least 50%, or at least 80%, and up to about 99.99% (by mass) water. A hydrogel can be regarded as a material which is mostly water, yet behaves like a solid or semi-solid due to a three-dimensional crosslinked solid-like network, made of polymeric chains (e.g., collagen chains), within the liquid dispersing medium. The polymeric chains are inter-connected (crosslinked) by chemical bonds (covalent, hydrogen and ionic/complex/metallic bonds, typically covalent bonds).

Herein throughout, whenever polymeric chains or a polymeric material is described, it encompasses a polymeric biological materials (e.g., macromolecules) such as peptides, proteins, oligonucleotides and nucleic acids.

Hydrogels may take a physical form that ranges from soft, brittle and weak to hard, elastic and tough material. Soft hydrogels may be characterized by rheological parameters including elastic and viscoelastic parameters, while hard hydrogels are suitably characterized by tensile strength parameters, elastic, storage and loss moduli, as these terms are known in the art.

The softness/hardness of a hydrogel is governed inter alia by the chemical composition of the polymer chains, the “degree of cross-linking” (number of interconnected links between the chains), the aqueous media content and composition, and temperature.

According to some of any of the embodiments described herein, the bioink composition features, when hardened, storage modulus (G′) that ranges from about 100 Pa to about 50,000 Pa, or from about 1,000 Pa to about 50,000 Pa, or from about 100 Pa to about 40,000 Pa, or from about 1,000 Pa to about 40,000 Pa, or from about 100 Pa to about 30,000 Pa, or from about 1,000 Pa to about 30,000 Pa, or from about 100 Pa to about 25,000 Pa, or from about 1,000 Pa to about 25,000 Pa, or from about 1,000 Pa to about 20,000 Pa, or from about 100 Pa to about 20,000 Pa, or from about 1,000 Pa to about 20,000 Pa, or from about 5,000 Pa to about 30,000 Pa, or from about 5,000 Pa to about 25,000 Pa, or from about 10,000 Pa to about 30,000 Pa or from about 10,000 Pa to about 25,000 Pa, including any intermediate values and subranges therebetween.

A hydrogel, according to some embodiments of the present invention, may contain macromolecular polymeric and/or fibrous elements which are not chemically connected to the main crosslinked network but are rather mechanically intertwined therewith and/or immersed therein. Such macromolecular fibrous elements can be woven (as in, for example, a mesh structure), or non-woven, and can, in some embodiments, serve as reinforcing materials of the hydrogel's fibrous network. Non-limiting examples of such macromolecules include polycaprolactone, gelatin, cross-linked gelatin formed of, for example, gelatin methacrylate, alginate, cross-linked alginate formed of, for example, alginate methacrylate, chitosan, cross-linked chitosan formed of, for example, chitosan methacrylate, glycol chitosan, cross-linked glycol chitosan from of, for example, glycol chitosan methacrylate, hyaluronic acid (HA), cross-linked hyaluronic acid form of, for example, HA methacrylate, and other cross-linked or non-crosslinked natural or synthetic polymeric chains and the likes. Alternatively, or in addition, such macromolecules are chemically connected to the main crosslinked network of the hydrogel, for example, by acting as a cross-linking agent, or by otherwise forming a part of the three-dimensional network of the hydrogel.

In some embodiments, the hydrogel is porous and in some embodiments, at least a portion of the pores in the hydrogel are nanopores, having an average volume at the nanoscale range.

According to some of any of the embodiments described herein, the curable formulation further comprises one or more additional materials, including, for example, one or more non-curable materials and/or one or more biological components or materials.

According to some of any of the embodiments described herein, the printing media (the building material, as described herein) comprises one or more additional materials, including, for example, one or more non-curable materials and/or one or more biological components.

Other curable materials that can be included in the curable formulation, optionally in combination with a curable collagen as described herein, can be any photocurable biocompatible materials.

In some embodiments, the biocompatible photocurable material is or comprises a hydrogel, as defined herein, which can form a hardened modeling material, typically upon further cross-linking and/or co-polymerization, when exposed to a curing condition at which the cross-linking and/or co-polymerization reaction occurs. Such curable materials are also referred to herein as hydrogel curable materials or as hydrogel-forming materials.

In some of any of the embodiments described herein, a curable material is or comprises a hydrogel forming material, as defined herein, which can form a hydrogel as a hardened modeling material, typically upon cross-linking, entanglement, polymerization and/or co-polymerization, when exposed to a curing condition at which the cross-linking, polymerization and/or co-polymerization, and/or entanglement reaction occurs. Such curable materials are also referred to herein as hydrogel-forming curable materials or as gel-forming materials.

The hydrogel, according to embodiments of the present invention, can be of biological origin or synthetically prepared.

According to some embodiments of the present invention, the hydrogel is biocompatible, and is such that when a biological moiety is impregnated or accumulated therein, an activity of the biological moiety is maintained, that is, a change in an activity of the biological moiety is no more than 30%, or no more than 20%, or no more than 10%, compared to an activity of the biological moiety in a physiological medium.

Exemplary polymers or co-polymers usable for forming a hydrogel according to the present embodiments include polyacrylates, polymethacrylates, polyacrylamides, polymethacrylamides, polyvinylpyrrolidone and copolymers of any of the foregoing. Other examples include polyethers, polyurethanes, and poly(ethylene glycol), functionalized by cross-linking (e.g., curable) groups or usable in combination with compatible cross linking agents.

Some specific, non-limiting examples, include: poly(2-vinylpiridine), poly(acrylic acid), poly(methacrylic acid), poly(N-isopropylacrylamide), poly(N,N′-methylenbisacrylamide), poly(N—(N-propyl) acrylamide), poly(methacyclic acid), poly(2-hydroxyacrylamide), poly(ethylene glycol) acrylate, poly(ethylene glycol) methacrylate, and polysaccharides such as hyaluronic acid, dextran, alginate, agarose, and the like, and any co-polymer of the foregoing.

Hydrogel precursors (hydrogel-forming materials) forming such polymeric chains are contemplated, including any combination thereof.

Hydrogels are typically formed of, or are formed in the presence of, di- or tri- or multi-functional monomers, oligomer or polymers, which are collectively referred to as hydrogel precursors or hydrogel-forming agents or hydrogen-forming materials, having two, three or more polymerizable groups. The presence of more than one polymerizable group renders such precursors cross-linkable, and allow the formation of the three-dimensional network.

Exemplary cross-linkable monomers include, without limitation, the family of di- and triacrylates monomers, which have two or three polymerizable functionalities, one of which can be regarded as a cross-linkable functional group. Exemplary diacrylates monomers include, without limitation, methylene diacrylate, and the family of poly(ethylene glycol), dimethacrylate (nEGDMA). Exemplary triacrylates monomers include, without limitation, trimethylolpropane triacrylate, pentaerythritol triacrylate, tris(2-hydroxy ethyl) isocyanurate triacrylate, isocyanuric acid tris(2-acryloyloxyethyl) ester, ethoxylated trimethylolpropane triacrylate, pentaerythrityl triacrylate and glycerol triacrylate, phosphinylidynetris(oxyethylene) triacrylate.

In some of any of the embodiments described herein, a curable material, whether monomeric or oligomeric, can be a mono-functional curable material or a multi-functional curable material.

Curable materials usable in the field of bioprinting are predominantly based on either naturally derived materials, including, for example, Matrigel, Alginate, Pectin, Xanthan gum, Gelatin, Chitosan, Fibrin, Cellulose and Hyaluronic acid, which can be isolated from animal or human tissues, or recombinantly-generated, or synthetically-prepared materials, including, for example, poly(ethyleneglycol), PEG, gelatin methacrylate; GelMA, poly(propylene oxide); PPO, poly(ethylene oxide); PEO; poly(ethyleneglycol) methacrylate (PEG-MA), polyethyleneglycol-diacrylate (PEG-DA), polyglutamic acid, PLGA/PLLA, poly(dimethyl siloxane); Nanocellulose; Pluronic F127, short di-peptides (FF), Fmoc-peptide-based hydrogels such as Fmoc-FF-OH, Fmoc-FRGD-OH, Fmoc-RGDF-OH, Fmoc-2-Nal-OH, Fmoc-FG-OH, and thermoplastic polymers such as Polycaprolactone (PCL), Polylactic acid (PLA) or Poly(D,L-lactide-co-glycolide).

Exemplary curable materials usable in the context of the present embodiments include, but are not limited to, Matrigel, Gelatin methacrylate (GelMA), Nanocellulose (nano-scaled structured materials which are UV-curable, including cellulose nanocrystals (CNC), cellulose nanofibrils (CNF), and bacterial cellulose (BC), also referred to as microbial cellulose), Pluronic® materials, including, for example, Pluronic F127 which is fluid at a low temperature forms a gel at a high temperature, above critical micellar concentration (CMC) and Pluronic F127-diacrylate (DA) which is UV-curable, Hyaluronic acid (HA), Acrylated hyaluronic acid (AHA), methacrylated hyaluronic (MAHA), Poly-(ethylene glycol) diacrylate acid (PEGDA), poly(ethyleneglycol)methacrylate (PEG-MA), Alginate, Xanthan gum, Pectin, Chitosan which can be crosslinked with a chemical agent such as Glutaraldehyde, Genipin or Sodium Tripolyphosphate (TPP).

According to some of any of the embodiments described herein, the curable material is or comprises a poly(alkylene glycol) such as a poly(ethylene glycol) that features one or more photocurable group(s), for example, one or more acrylic group(s) as described herein. In some of these embodiments, the curable material can be, for example, a poly(alkylene glycol) (meth)acrylate such as a poly(ethylene glycol) (meth)acrylate, and/or a poly(alkylene glycol) di(meth)acrylate such as a poly(ethylene glycol) di(meth)acrylate, and/or a copolymer that comprises the foregoing, for example, poly caprolactone (meth)acrylate and/or di(meth)acrylate/poly(ethylene glycol); poly(lactic acid) (meth)acrylate and/or di(meth)acrylate/poly(ethylene glycol); poly(lactic acid co-glycolic acid) (meth)acrylate and/or di(meth)acrylate/poly(ethylene glycol), including any combination of the foregoing.

According to some of any of the embodiments described herein, the curable material is or comprises a poly(alkylene glycol) that features at least one (meth)acrylic group at its terminus, that is, it is a polymer or copolymer of poly(alkylene glycol) that features at least one (meth)acrylic group at its terminus.

According to some of any of the embodiments described herein, a poly(ethylene glycol) that features curable group(s) or a co-polymer thereof has an average molecular weight of at least 500 grams/mol or at least 700 grams/mol. In some embodiments, the average molecular weight is lower than 4,000, or lower than 3,000 or lower than 2,000, or lower than 1,000 grams/mol. In some embodiments, the average molecular weight ranges from 500 to 30,000, or from 500 to 20,000, or from 50 to 10,000, or from 500 to 5,000, or from 500 to 4,000, or from 500 to 3,000, or from 500 to 2,000, grams/mol, including any intermediate values and subranges therebetween. In some embodiments, the average molecular weight ranges from 3,000 to about 30,000, or from 3,000 to about 20,000, or from 3,000 to about 10,000, grams/mol, including any intermediate values and subranges therebetween.

According to some of any of the embodiments described herein, a concentration of each of the curable materials in the formulation ranges from 1 to 30, or from 1 to 20, or from 1 to 10, or from 5 to 20, or from 5 to 15, or from 10 to 20, or from 10 to 30, % by weight of the total weight of the composition, including any intermediate values and subranges therebetween.

In some of any of the embodiments described herein, a concentration of each of the curable materials as described herein in any of the respective embodiments and any combination thereof in the curable formulation ranges from 0.5 mg/mL to 50 mg/mL, or from 0.5 mg/mL to 20 mg/mL, or from 1 mg/mL to 50 mg/mL, or from 1 mg/mL to 40 mg/mL, or from 1 mg/mL to 30 mg/mL, or from 1 mg/mL to 20 mg/mL, or from 0.5 mg/mL to 10 mg/mL, or from 1 mg/mL to 10 mg/mL, including any intermediate values and subranges therebetween.

A concentration of the curable materials in a curable formulation containing the same can affect the rheological properties of the formulation and of the hardened material obtained upon curing (upon exposure to a curing condition such as, for example, irradiation), and can be manipulated in accordance with the AM methodology and conditions employed and desired properties of the final object or a portion thereof.

According to some of any of the embodiments described herein, the curable formulation further comprises a carrier and in some embodiments, the carrier is an aqueous carrier.

The aqueous carrier can be water, a buffer featuring pH in a range of from about 2 to about 10, or from about 2 to about 9, or from about 3 to about 9, or from about 3 to about 8, a basic aqueous solution, a neutral aqueous solution or an acidic aqueous solution.

The aqueous carrier can comprise salts and other water-soluble materials at varying concentrations. In some embodiments, a concentration of a salt in the carrier ranges from about 0.1 mM to about 0.2 M, or from about 0.1 mM to about 0.1 M, or from about 0.1 mM to about 100 mM, or from about 0.1 mM to about 50 mM, or from about 0.1 mM to about 20 mM, including an intermediate values and subranges therebetween.

In some embodiments, the aqueous carrier comprises salts at physiologically acceptable concentrations, such that the formulation features osmolarity around a physiological osmolarity.

In some embodiments the aqueous carrier comprises a phosphate salt, for example, a sodium phosphate monobasic (NaH₂PO₄) and/or a sodium phosphate dibasic (sodium hydrogen phosphate; Na₂HPO₄). In some embodiments, the total concentration of the phosphate salt(s) in the formulation is about 0.1 M.

In some embodiments, the aqueous carrier comprises NaCl or any other physiologically acceptable salt.

In some embodiments, the aqueous carrier comprises a phosphate buffer and in some embodiments, the aqueous carrier comprises a phosphate buffer saline, which comprises sodium phosphate monobasic and/or sodium phosphate dibasic and NaCl.

The phosphate buffer saline (PBS) can be a commercially available PBS (e.g., DPBS) or a custom-made buffer featuring a desirable pH and/or osmolarity.

In exemplary embodiments, the aqueous carrier comprises a phosphate buffer that comprises a phosphate sodium salt as described herein at a concentration of about 0.1M and NaCl at a concentration of from about 0.01 mM to about 200 mM, including any intermediate value and subranges therebetween.

Any other buffers are also usable in the context of the present embodiments.

In some of any of the embodiments described herein, the aqueous carrier comprises an acid.

In some embodiments, a concentration of the acid is lower than 100 mM, and can be, for example, of from 0.1 mM to 50 mM, or from 0.1 mM to 30 mM, or from 0.1 mM to 40 mM, or from 0.1 mM to 30 mM, or from 1 to 30 mM, or from 10 to 30 Mm, including any intermediate values and subranges therebetween.

The acid can be an inorganic acid (e.g., HCl) or an organic acid, preferably which is water soluble at the above-indicated concentrations (e.g., acetic acid).

In some of any of the embodiments described herein, the aqueous carrier comprises a culturing medium. The culturing medium can be a commercially available culturing medium or a custom-made culturing medium. The culture medium can be any liquid medium which allows at least cell survival. Such a culture medium can include, for example, salts, sugars, amino acids and minerals in the appropriate concentrations and with various additives and those of skills in the art are capable of determining a suitable culture medium to specific cell types. Non-limiting examples of such culture medium include, phosphate buffered saline, DMEM, MEM, RPMI 1640, McCoy's 5A medium, medium 199 and IMDM (available e.g., from Biological Industries, Beth Ha'emek, Israel; Gibco-Invitrogen Corporation products, Grand Island, NY, USA).

The culture medium may be supplemented with various antibiotics (e.g., Penicillin and Streptomycin), growth factors or hormones, specific amino acids (e.g., L-glutamin) cytokines and the like.

According to some of any of the embodiments described herein, the curable formulation features a pH that ranges from about 2 to about 9, or from about 3 to about 9, or from about 3 to about 8.5, or from about 3 to about 8, or from about 3.5 to about 9, or from about 3.5 to about 8.5, or from about 3.5 to about 8, or from about 4 to about 8.5, or from about 4 to about 8, or from about 4.5 to about 8.5, or from about 4.5 to about 8, or from about 5 to about 8.5, or from about 5 to about 8, or from about 5.5 to about 8.5, or from about 5.5 to about 8, or from about 6 to about 8, including any intermediate values and subranges therebetween.

According to some of any of the embodiments described herein, the curable formulation features a shear-thinning behavior (e.g., at room temperature, for example, of from 20 to 25 °C) and is a shear-thinning composition.

The term “shear-thinning” describes a property of a fluidic material that is reflected by a decrease in its viscosity (increase in its fluidity) upon application of shear forces (under shear strain), at an indicated temperature, when determined using a rheometer as described in the Examples section that follows.

In some of the present embodiments, a shear-thinning material is such that exhibits a significant, e.g., at least 100%, reduction in its shear modulus upon increasing the shear strain from about 1% to above 50%. Shear-thinning materials therefore exhibit a shear-dependent viscosity profile.

According to some of any of the embodiments described herein, the curable formulation features a fast recovery rate upon a change in the applied shear force (a fast shear recovery).

According to some of any of the embodiments described herein, the curable formulation features a change of no more than 6%, or of no more than 10% upon shear rest (zero shear force) of 1 minute, 2 minutes, 3 minutes, 4 minutes, 5 minutes and even 10 minutes.

According to some of any of the embodiments described herein, the curable formulation features at least 80% recovery, or at least 85% recovery, or at least 90% recovery, or at least 92% recovery, of its viscosity upon increasing the shear rate from about 0 1/sec or 1 1/sec to above 50 1/sec, for a time period of at least 1 minute (e.g., from about 60 seconds to about 120 seconds, e.g., about 100 seconds).

According to some of any of the embodiments described herein, the curable formulation features a viscosity of no more than 200 centipoises, or no more than 250 centipoises, at a shear rate of 10 1/sec, at room temperature, as described herein, when determined using a rheometer as described in the Examples section that follows.

As discussed herein, the curable formulation according to any of the respective embodiments, is usable as one or more modeling material formulation(s) for an additive manufacturing process (e.g., bioprinting), as described in further detail hereinafter.

Altogether, components that are described herein as included in a bioink composition, can be included either in the same modeling formulation or altogether within the bioprinting media, the building material, and can be optionally divided between two or more modeling material formulations, as long as the additive manufacturing requirements are met.

According to some of any of the embodiments described herein, the printing media (building material) in general or the curable formulation as described herein in particular further comprises a biological component or material other than the biocompatible curable material(s) (collectively referred to herein also as a biological material).

Biological components or materials that can be included in one or more curable (e.g., modeling material) formulations as described herein include cellular components, including, for example, culturing cells, and other cellular components such as cytokines, chemokines, growth factors; as well as other biological components such as proteins, agents that act to increase cell attachment, cell spreading, cell proliferation, cell differentiation and/or cell migration; an amino acid, peptides, polypeptides, proteins, DNA, RNA, lipids and/or proteoglycans.

Cells may comprise a heterogeneous population of cells or alternatively the cells may comprise a homogeneous population of cells. Such cells can be for example stem cells (such as embryonic stem cells, bone marrow stem cells, cord blood cells, mesenchymal stem cells, adult tissue stem cells), progenitor cells, or differentiated cells such as chondrocytes, osteoblasts, connective tissue cells (e.g., fibrocytes, fibroblasts and adipose cells), endothelial and epithelial cells. The cells may be naïve or genetically modified.

According to one embodiment of this aspect of the present invention, the cells are mammalian in origin.

Furthermore, the cells may be of autologous origin or non-autologous origin, such as postpartum-derived cells (as described in U.S. application Ser. Nos. 10/887,012 and 10/887,446). Typically, the cells are selected according to the desired application.

Suitable proteins which can be used include, but are not limited to, extracellular matrix proteins [e.g., fibrinogen, collagen, fibronectin, vimentin, microtubule-associated protein 1D, Neurite outgrowth factor (NOF), bacterial cellulose (BC), laminin and gelatin], cell adhesion proteins [e.g., integrin, proteoglycan, glycosaminoglycan, laminin, intercellular adhesion molecule (ICAM) 1, N-CAM, cadherin, tenascin, gicerin, RGD peptide and nerve injury induced protein 2 (ninjurin2)], growth factors [epidermal growth factor, transforming growth factor-α, fibroblast growth factor-acidic, bone morphogenic protein, fibroblast growth factor-basic, erythropoietin, thrombopoietin, hepatocyte growth factor, insulin-like growth factor-I, insulin-like growth factor-II, Interferon-β, platelet-derived growth factor, Vascular Endothelial Growth Factor and angiopeptin], cytokines [e.g., M-CSF, IL-1beta, IL-8, beta-thromboglobulin, EMAP-II, G-CSF and IL-10], proteases [pepsin, low specificity chymotrypsin, high specificity chymotrypsin, trypsin, carboxypeptidases, aminopeptidases, proline-endopeptidase, Staphylococcus aureus V8 protease, Proteinase K (PK), aspartic protease, serine proteases, cysteine proteases, metalloproteases, ADAMTS17, tryptase-gamma, and matriptase-2] and protease substrates.

In addition, calcium phosphate materials, such as hydroxyapatite, for example, in a form of particles, can be used, including, but not limited to, nanoHA and nanoTCP. When the AM process involves dispensing the curable formulation through dispensing heads, the particles size should be compatible with the dispensing heads so as to avoid clogging.

Non-curable materials, other than the biological materials as described herein, that can be included in one or more curable (e.g., modeling material) formulations as described herein can be materials that impart a certain property to the formulation or to the hardened formulation or material and to the part of the object formed thereby. Such a property can be a physical property (e.g., an optical property such as transparency or opacity, color, a spectral property, heat resistance, electrical property and the like), or a mechanical or rheological property such as viscosity, elasticity, storage modulus, loss modulus, stiffness, hardness, and the like. Alternatively, or in addition, non-curable materials can be such that provide a biological function, for example, therapeutically active agents.

Exemplary non-curable materials include thixotropic agents, reinforcing agents, toughening agents, fillers, colorants, pigments, dye substances (e.g., as described herein), etc.

An exemplary non-curable material includes titanium dioxide.

An exemplary non-curable material includes oxidized cellulose.

According to some of any of the embodiments described herein, one or more of the curable (e.g., modeling material) formulations comprises hyaluronic acid.

According to some of any of the embodiments described herein, one or more of the curable (e.g., modeling material) formulations comprises hyaluronic acid featuring a curable group as defined herein.

According to some of any of the embodiments described herein, one or more of the curable (e.g., modeling material) formulations comprises one or more biological components or materials such as, but not limited to, cells, growth factors, peptides, heparan sulfate and fibronectin.

According to some of any of the embodiments described herein, one or more of the curable (e.g., modeling material) formulations comprises one or more agents that modify a mechanical property of the formulation and/or the object, as described herein, such as, but not limited to, alginate, hyaluronic acid, fibrinogen, elastin, peptides and a thixotropic agent (e.g., Crystaline nano cellulose (CNC)), oxidized cellulose, titanium dioxide, Clay mineral and carbon nanotubes.

In some of any of the embodiments described herein the curable formulation further comprises a thixotropic agent, as defined herein.

Herein throughout, the term “thixotropic” describes a property of a fluidic compound or material that is reflected by a time-dependent shear-thinning, that is its viscosity is decreased in correlation with the time at which shear forces are applied, and returns back to its original value when application of shear forces is ceased. In some of the present embodiments, a thixotropic material or agent is such that exhibits or imparts a significant, e.g., at least 100%, reduction in shear modulus under 50% strain.

In some of any of the embodiments described herein, the curable formulation further comprises a gel-forming agent, for example, a hydrogel-forming agent as described herein.

In some of any of the embodiments described herein, the curable formulation further comprises a biological component or material as described herein.

In some of any of the embodiments described herein, the curable formulation further comprises one or more curable or non-curable materials as described herein in any of the respective embodiments.

According to some of any of the embodiments described herein, the curable formulation further comprises one or more biological components such as, but not limited to, hyaluronic acid (including curable HA), cells, growth factors, peptides, heparan sulfate and/or fibronectin.

According to some of any of the embodiments described herein the curable formulation further comprises one or more agents that modify a mechanical property of the formulation and/or the object, such as, but not limited to, alginate, hyaluronic acid, fibrinogen, elastin, peptides and a thixotropic agent (e.g., Crystalline nano cellulose (CNC)).

In some of any of the embodiments described herein, all the curable materials in the building material are cured under the same curing condition, and are photocurable.

### Additive Manufacturing:

According to an aspect of some embodiments of the present invention, there is provided a process (a method) of additive manufacturing (AM) of a three-dimensional object. According to embodiments of this aspect, the method is effected by sequentially forming a plurality of layers in a configured pattern corresponding to the shape of the object, thereby forming the object. According to some embodiments of this aspect, formation of each layer is effected by dispensing at least one uncured building material, and exposing the dispensed building material to a curing condition to thereby form a hardened (cured) material. According to some embodiments of this aspect, formation of each layer is effected by exposing a layer of uncured building material to a curing condition, and the method is effected by sequentially exposing, in a layer-wise manner, an uncured building material to a curing condition, whereby the exposure to the curing condition is effected in a configured pattern corresponding to the shape of the object.

Herein throughout, the phrase “building material” encompasses the phrases “uncured building material” or “uncured building material formulation” and collectively describes the materials that are dispensed by sequentially forming the layers, as described herein. This phrase encompasses uncured materials which form the final object, namely, one or more uncured modeling material formulation(s), and optionally also uncured materials used to form a support, namely uncured support material formulations. The building material can also include non-curable materials that preferably do not undergo (or are not intended to undergo) any change during the process, for example, biological materials or components (other than a curable collagen as described herein) and/or other agents or additives as described herein.

The building material that is used to sequentially form the layers as described herein is also referred to herein interchangeably as “printing medium” or “bioprinting medium” or “bioink”.

An uncured building material can comprise one or more modeling material formulations, and can be utilized such that different parts of the object are made upon hardening (e.g., curing) of different modeling formulations, and hence are made of different hardened (e.g., cured) modeling materials or different mixtures of hardened (e.g., cured) modeling materials.

The method of the present embodiments manufactures three-dimensional objects in a layerwise manner by forming a plurality of layers in a configured pattern corresponding to the shape of the object.

Each layer is formed by an additive manufacturing apparatus which scans a two-dimensional surface and patterns it. While scanning, the apparatus visits a plurality of target locations on the two-dimensional layer or surface, and decides, according to a pre-set algorithm, for each target location or a group of target locations, whether or not the target location or group of target locations is to be occupied by a building material, and which type of a building material is to be delivered thereto. The decision is made according to a computer image of the surface.

When the AM is by three-dimensional inkjet printing, an uncured building material, as defined herein, is dispensed from a dispensing head having a set of nozzles to deposit building material in layers on a supporting structure. The AM apparatus thus dispenses building material in target locations which are to be occupied and leaves other target locations void. The apparatus typically includes a plurality of dispensing heads, each of which can be configured to dispense a different building material (for example, different modeling material formulations, each containing a different biological component; or each containing a different curable material; or each containing a different concentration of a curable material, and/or different support material formulations). Thus, different target locations can be occupied by different building materials (e.g., a modeling formulation and/or a support formulation, as defined herein).

The final three-dimensional object is made of the hardened modeling material or a combination of hardened modeling materials or a combination of hardened modeling material/s and support material/s or modification thereof (e.g., following curing). All these operations are well-known to those skilled in the art of additive manufacturing (also known as solid freeform fabrication).

In some exemplary embodiments of the invention an object is manufactured by dispensing a building material that comprises two or more different modeling material formulations, each modeling material formulation from a different dispensing head of the AM apparatus. The modeling material formulations are optionally and preferably deposited in layers during the same pass of the dispensing heads. The modeling material formulations and/or combination of formulations within the layer are selected according to the desired properties of the object.

An exemplary process according to some embodiments of the present invention starts by receiving 3D printing data corresponding to the shape of the object. The data can be received, for example, from a host computer which transmits digital data pertaining to fabrication instructions based on computer object data, e.g., in a form of a Standard Tessellation Language (STL) or a StereoLithography Contour (SLC) format, Virtual Reality Modeling Language (VRML), Additive Manufacturing File (AMF) format, Drawing Exchange Format (DXF), Polygon File Format (PLY), Digital Imaging and Communications in Medicine (DICOM) or any other format suitable for Computer-Aided Design (CAD).

The process continues by dispensing the building material as described herein in layers, on a receiving medium, using one or more dispensing (e.g., printing) heads, according to the printing data.

The dispensing can be in a form of droplets, or a continuous stream, depending on the additive manufacturing methodology employed and the configuration of choice.

The receiving medium can be a tray of a printing system, or a supporting article or medium made of, or coated by, a biocompatible material, such as support media or articles commonly used in bioprinting, or a previously deposited layer.

In some embodiments, the receiving medium comprises a sacrificial hydrogel or other biocompatible material as a mold to embed the printed object, and is thereafter removed by chemical, mechanical or physical (e.g., heating or cooling) means. Such sacrificial hydrogels can be made of, for example, a Pluronic material or of Gelatin.

Once the uncured building material is dispensed on the receiving medium according to the 3D data, the method optionally and preferably continues by hardening the dispensed formulation(s). In some embodiments, the process continues by exposing the deposited layers to a curing condition. Preferably, the curing condition is applied to each individual layer following the deposition of the layer and prior to the deposition of the previous layer.

As used herein throughout, the term “curing” describes a process in which a formulation is hardened. The hardening of a formulation typically involves an increase in a viscosity of the formulation and/or an increase in a storage modulus of the formulation (G′). In some embodiments, a formulation which is dispensed as a liquid becomes solid or semi-solid (e.g., gel) when hardened. A formulation which is dispensed as a semi-solid (e.g., soft gel) becomes solid or a harder or stronger semi-solid (e.g., strong gel) when hardened.

The term “curing” as used herein encompasses, for example, polymerization of monomeric and/or oligomeric materials and/or cross-linking of polymeric chains (either of a polymer present before curing or of a polymeric material formed in a polymerization of the monomers or oligomers). The product of a curing reaction is therefore typically a polymeric material and/or a cross-linked material. This term, as used herein, encompasses also partial curing, for example, curing of at least 20% or at least 30% or at least 40% or at least 50% or at least 60% or at least 70% of the formulation, in addition to curing of 100% of the formulation.

Herein, the phrase “a condition that affects curing” or “a condition for inducing curing”, which is also referred to herein interchangeably as “curing condition” or “curing inducing condition” describes a condition which, when applied to a formulation that contains a curable material, induces a curing as defined herein. Such a condition can include, for example, application of a curing energy, as described hereinafter to the curable material(s), and/or contacting the curable material(s) with chemically reactive components such as catalysts, co-catalysts, and activators.

When a condition that induces curing comprises application of a curing energy, the phrase “exposing to a curing condition” and grammatical diversions thereof means that the dispensed layers are exposed to the curing energy and the exposure is typically performed by applying a curing energy to the dispensed layers.

A “curing energy” typically includes application of radiation (irradiation) or application of heat.

The radiation can be electromagnetic radiation (e.g., ultraviolet or visible light), or electron beam radiation, or ultrasound radiation or microwave radiation, depending on the materials to be cured. The application of radiation (or irradiation) is effected by a suitable radiation source. For example, an ultraviolet or visible or infrared or Xenon or mercury or lamp, or LED source, can be employed, as described herein.

A curable material or system that undergoes curing upon exposure to radiation is referred to herein interchangeably as “photopolymerizable” or “photoactivatable” or “photocurable”.

When the curing energy comprises heat, the curing is also referred to herein and in the art as “thermal curing” and comprises application of thermal energy. Applying thermal energy can be effected, for example, by heating a receiving medium onto which the layers are dispensed or a chamber hosting the receiving medium, as described herein. In some embodiments, the heating is effected using a resistive heater.

In some embodiments, the heating is effected by irradiating the dispensed layers by heat-inducing radiation. Such irradiation can be effected, for example, by means of an IR lamp or Xenon lamp, operated to emit radiation onto the deposited layer.

In some embodiments, heating is effected by infrared radiation applied by a ceramic lamp, for example, a ceramic lamp that produces infrared radiation of from about 3 μm to about 4 μm, e.g., about 3.5 μm.

A curable material or system that undergoes curing upon exposure to heat is referred to herein as “thermally-curable” or “thermally-activatable” or “thermally-polymerizable”.

In some of any of the embodiments described herein, hardening the dispensed formulation(s) comprises exposing the dispensed formulation to a curing condition which is irradiation (illumination), as described herein.

In some embodiments, the exposure to a curing condition (irradiation) is for a short time period, for example, a time period of less than 3 minutes, less than 300 seconds, for example, of from 10 seconds to 240 seconds, or from 10 seconds to 120 seconds, to from 10 seconds to 60 seconds, including an intermediate values and subranges therebetween.

In some embodiments, exposing to the curing condition (irradiation) is for time period that ranges from 1 second to 120 second.

In some embodiments, the irradiation is at wavelength within the UV-vis range.

In some embodiments, the irradiation is at wavelength in a range of from about 300 to about 800, or from about 300 to about 600, or from about 300 to about 500, or from about 350 to about 350, nm, including any intermediate values and subranges therebetween. In exemplary embodiments, the irradiation is at 385 nm.

In some embodiments, the irradiation is at level that ranges from about 1 to about 150, or from about 1 to about 130, or from about 1 to about 100, or from about 10 to about 150, or from about 10 to about 130, or from about 10 to about 100, or from about 50 to about 150, or from about 50 to about 130, or from about 50 to about 100, or from about 1 to about 50 or from about 1 to about 30 or from about 1 to about 20, from about 1 to about 10, from about 1 to about 9, from about 1 to about 10 mW/cm², including any intermediate values and subranges therebetween.

In some of any of the embodiments described herein, the exposure, controls the amount of energy delivered by the light source to the printed layer. In some embodiments, the exposure is at a level that ranges from about 1 to about 150, or from about 1 to about 130, or from about 1 to about 100, or from about 10 to about 150, or from about 10 to about 130, or from about 10 to about 100, or from about 50 to about 150, or from about 50 to about 130, or from about 50 to about 100, or from about 1 to about 50 or from about 1 to about 30 or from about 1 to about 20, from about 1 to about 10, from about 1 to about 9, from about 1 to about 10 mJ/cm², including any intermediate values and subranges therebetween.

In some embodiments, printing is controlled by one or more parameters comprising exposure energy, exposure time, layer thickness, concentration of the photo initiator or the concentration photo blocker.

In some of any of the embodiments described herein, the method further comprises exposing the cured modeling material formulation(s) either before or after removal of a support material formulation, if such has been included in the building material, to a post-treatment condition. The post-treatment condition is typically aimed at further hardening the cured modeling material(s). In some embodiments, the post-treatment hardens a partially-cured formulation to thereby obtain a completely cured formulation.

In some embodiments, the post-treatment is effected by exposure to heat or radiation, as described in any of the respective embodiments herein.

Some embodiments contemplate the fabrication of an object by dispensing different formulations from different dispensing heads. These embodiments provide, inter alia, the ability to select formulations from a given number of formulations and define desired combinations of the selected formulations and their properties.

According to the present embodiments, the spatial locations of the deposition of each formulation with the layer are defined, either to effect occupation of different three-dimensional spatial locations by different formulations, or to effect occupation of substantially the same three-dimensional location or adjacent three-dimensional locations by two or more different formulations so as to allow post deposition spatial combination of the formulations within the layer.

The present embodiments thus enable the deposition of a broad range of material combinations, and the fabrication of an object which may consist of multiple different combinations of modeling material formulations, in different parts of the object, according to the properties desired to characterize each part of the object.

A system utilized in additive manufacturing may include a receiving medium and one or more dispensing heads. The receiving medium can be, for example, a fabrication tray that may include a horizontal surface to carry the material dispensed from the printing head. In some embodiments, the receiving medium is made of, or coated by, a biocompatible material, as described herein.

The dispensing head may be, for example, a printing head having a plurality of dispensing nozzles arranged in an array of one or more rows along the longitudinal axis of the dispensing head. The dispensing head may be located such that its longitudinal axis is substantially parallel to the indexing direction.

The additive manufacturing system may further include a controller, such as a microprocessor to control the AM process, for example, the movement of the dispensing head according to a pre-defined scanning plan (e.g., a CAD configuration converted to a Standard Tessellation Language (STL) format and programmed into the controller). The dispensing head may include a plurality of jetting nozzles. The jetting nozzles dispense material onto the receiving medium to create the layers representing cross sections of a 3D object.

In addition to the dispensing head, there may be a source of curing energy, for curing the dispensed building material. The curing energy is typically radiation, for example, UV radiation or heat radiation. Alternatively, there may be means for providing a curing condition other than electromagnetic or heat radiation, for example, means for cooling the dispensed building material or for contacting it with a reagent that promotes curing.

Additionally, the AM system may include a leveling device for leveling and/or establishing the height of each layer after deposition and at least partial solidification, prior to the deposition of a subsequent layer.

According to the present embodiments, the additive manufacturing method described herein is for bioprinting a biological object.

As used herein, “bioprinting” means practicing an additive manufacturing process while utilizing one or more bio-ink formulation(s) that comprise(s) biological components, as described herein, via methodology that is compatible with an automated or semi-automated, computer-aided, additive manufacturing system as described herein (e.g., a bioprinter or a bioprinting system).

Herein throughout, the phrase “modeling material formulation”, which is also referred to herein interchangeably as “modeling formulation” or “modeling material composition” or “modeling composition”, or simply as a “formulation”, or a “composition”, describes a part or all of the uncured building material (printing medium) which is dispensed so as to form the final object, as described herein. The modeling formulation is an uncured modeling formulation, which, upon exposure to a curing condition, forms the object or a part thereof.

In the context of bioprinting, an uncured building material comprises at least one modeling formulation that comprises one or more biocompatible or biological components or materials (e.g., a curable collagen as described herein), and is also referred to herein and in the art as “bioink” or “bioink formulation” or “bioink composition”.

In some embodiments, the bioprinting comprises sequential formation of a plurality of layers of the uncured building material in a configured pattern, preferably according to a three-dimensional printing data, as described herein. At least one, and preferably most or all, of the formed layers (before hardening or curing) comprise(s) one or more biological component(s) as described herein (e.g., a curable Collagen as described herein). Optionally, at least one of the formed layers (before hardening or curing) comprises one or more non-biological curable materials, and/or non-curable biological or non-biological components, preferably biocompatible materials which do not interfere (e.g., adversely affect) with the biological and/or structural features of the biological components (e.g., collagen) in the printing medium and/or bio-ink.

In some embodiments, the components in the bioink or the printing medium, e.g., non-curable and curable materials, and/or the curing condition applied to effect curing, are selected such that they do not significantly affect structural and/or functional properties of the biological components in the bio-ink or printing medium.

In some of any of the embodiments described herein, the building material (e.g., the printing medium) comprises modeling material formulation(s) (e.g., a bioink composition as described herein) and optionally support material formulation(s), and all are selected to include materials or combination of materials that do not interfere with the biological and/or structural features of the biological components.

In some of any of the embodiments described herein, the bioprinting method is configured to effect formation of the layers under conditions that do not significantly affect structural and/or functional properties of the biological components in the bioink composition.

In some embodiments, a bioprinting system for effecting a bioprinting process/method as described herein is configured so as to allow formation of the layers under conditions that do not significantly affect structural and/or functional properties of the biological components in the bio-ink.

In some of any of the embodiments described herein, the additive manufacturing (e.g., bioprinting) process and system are configured such that the process parameters (e.g., temperature, shear forces, shear strain rate) do not interfere with (do not substantially affect) the functional and/or structural features of the biological components.

According to some of the present embodiments, the additive manufacturing is of a three-dimensional object featuring, in at least a portion thereof, a collagen-based material, and comprises dispensing at least one modeling material formulation to sequentially form a plurality of layers in a configured pattern corresponding to a shape of the object, wherein for at least a portion of the layers, the dispensing is of one or more modeling material formulation(s) that comprise the bioink composition as described herein in any of the respective embodiments and any combination thereof.

According to some of any of the embodiments described herein, the process further comprises exposing at least a portion of the dispensed layers to a curing condition which comprises curing energy, for example, light energy (irradiation, illumination).

According to some of any of the embodiments described herein, for at least a portion of the layers, the dispensing is further of a modeling material formulation that comprises an agent that modifies a mechanical and/or rheological and/or physical property of the formulation and/or of a respective portion of the object.

According to some of any of the embodiments described herein, or at least a portion of the layers, the dispensing is further of a modeling material formulation that comprises a biological material other than the biocompatible photocurable material (e.g., curable collagen) as described herein.

According to some of any of the embodiments described herein, the dispensing is at a temperature that ranges from −10 to 50 °C, or from −4 to 50 °C, or from −4 to 37 °C. In some embodiments, the temperature is at least 10 °C, or of at least 20 °C, or of 37 °C.

In some of any of the embodiments described herein, the additive manufacturing process (the bioprinting) is performed at a temperature of at least 10 °C, or of at least 20 °C, for example, at a temperature that ranges from about 10 to about 40 °C, preferably from about 10 °C to 37 °C, or from about 20 °C to 37 °C, or from about 20 °C to about 30 °C, or from about 20 °C to about 28 °C, or from about 20 °C to about 25 °C, including any intermediate values and subranges therebetween, or at room temperature, or at 37 °C.

In some of any of the embodiments described herein, the above-indicated temperatures/temperature ranges are the temperatures at which the building material (e.g., at least a modeling material formulation that comprises a biological component as described herein) are dispensed, that is, a temperature of a dispensing head in the AM system and/or a temperature at which the modeling material formulation is maintained prior to passing in the dispensing head and/or a temperature at which the modeling material formulation is maintained in the vat prior to curing.

In some of any of the embodiments described herein, the AM process is performed without cooling the AM system (e.g., without cooling the dispensing heads and/or a modeling material formulation and/or vat), to a temperature below room temperature, e.g., a temperature lower than 20 °C or lower than 10 °C, or lower than 5 °C (e.g., 4 °C).

In some of any of the embodiments described herein, the AM system is devoid of means for cooling the system or a part thereof (e.g., means for cooling the dispensing heads and/or the modeling material formulation and/or the vat), to a temperature below room temperature, e.g., a temperature lower than 20 °C or lower than 10 °C, or lower than 5 °C (e.g., 4 °C).

In some of any of the embodiments described herein, the additive manufacturing process (bioprinting) is performed while applying a shear force that does not adversely affect structural and/or functional properties of biological components (e.g., cells). Applying the shear force can be effected by passing the building material (e.g., at least a modeling material formulation that comprises a biological component as described herein) through the dispensing head, and is to be regarded also as subjecting the building material to shear force.

Some embodiments of the present invention allow to perform AM bioprinting processes under conditions that do not affect the functional and/or structural features of biological components included in the bio-ink (e.g., at low shear force and room temperature or a physiological temperature), while maintaining the required fluidity (a viscosity that imparts fluidity, e.g., lower than 10,000 centipoises or lower than 5,000 centipoises, or lower than 2,000 centipoises), and while further maintaining the curability of the dispensed building material.

The following describes exemplary AM bioprinting methodologies that are usable in the context of embodiments of the present invention.

A bioprinting method and a corresponding system can be any of the methods and systems known in the art for performing additive manufacturing, and exemplary such systems and methods are described hereinabove. A suitable method and system can be selected upon considering its printing capabilities, which include resolution, deposition speed, scalability, bio-ink compatibility and ease-of-use.

Exemplary suitable bioprinting systems usually contain a dispensing system (either equipped with temperature control module or at ambient temperature), and stage (a receiving medium), and a movement along the x, y and z axes directed by a CAD-CAM software. A curing source (e.g., a light or heat source) which applies a curing energy (e.g., by applying light or heat radiation) or a curing condition to the deposition area (the receiving medium) so as to promote curing of the formed layers and/or a humidifier, can also be included in the system. There are printers that use multiple dispensing heads to facilitate a serial dispensing of several materials.

Generally, bioprinting can be effected using any of the known techniques for additive manufacturing. The following lists some exemplary additive manufacturing techniques, although any other technique is contemplated.

### 3D Inkjet Printing:

3D Inkjet printing is a common type of 3D printer for both non-biological and biological (bioprinting) applications. Inkjet printers use thermal or acoustic forces to eject drops of liquid onto a substrate, which can support or form part of the final construct. In this technique, controlled volumes of liquid are delivered to predefined locations, and a high-resolution printing with precise control of (1) ink drops position, and (2) ink volume, which is beneficial in cases of microstructure-printing or when small amounts of bioreactive agents or drugs are added, is received. Inkjet printers can be used with several types of ink, for example, comprising multiple types of biological components and/or bioactive agents. Furthermore, the printing is fast and can be applied onto culture plates.

A bioprinting method that utilizes a 3D inkjet printing system can be operated using one or more bio-ink modeling material formulations as described herein, and dispensing droplets of the formulation(s) in layers, on the receiving medium, using one or more inkjet printing head(s), according to the 3D printing data.

### Extrusion Printing:

This technique uses continuous beads of material rather than liquid droplets. These beads of material are deposited in 2D, the stage (receiving medium) or extrusion head moves along the z axis, and the deposited layer serves as the basis for the next layer. The most common methods for biological materials extrusion for 3D bioprinting applications are pneumatic or mechanical dispensing systems

### Stereolithography (SLA) and Digital Light Processing (DLP):

SLA and DLP are additive manufacturing technologies in which an uncured building material in a bath or vat is converted into hardened material(s), layer by layer, by selective curing using a light source while the uncured material is later separated/washed from the hardened material. SLA is widely used to create models, prototypes, patterns, and production parts for a range of industries including for Bioprinting. DLP differs from laser-based SLA in that DLP uses a projection of ultraviolet (UV) light (or visible light) from a digital projector to flash a single image of the layer across the entire uncured material at once. One of the key components of DLP is a digital micromirror device (DMD) chip, which is typically composed of an array of reflective aluminum micromirrors that redirect incoming light from the UV source to project an image of a designed pattern. For achieving a high-resolution structure, parameters such as the curing time of each layer, layer thickness, and intensity of the UV light should be tuned, for example, by controlling the concentration and types of the curable materials, the photoabsorber and/or the photoinitiator.

### Laser-Assisted Printing:

Laser-assisted printing technique, in the version adopted for 3D bioprinting, is based on the principle of laser-induced forward transfer (LIFT), which was developed to transfer metals and is now successfully applied to biological material. The device consists of a laser beam, a focusing system, an energy absorbing/converting layer and a biological material layer (e.g., cells and/or hydrogel) and a receiving substrate. A laser assisted printer operates by shooting a laser beam onto the absorbing layer which convert the energy into a mechanical force which drives tiny drops from the biological layer onto the substrate. A light source is then utilized to cure the material on the substrate.

Laser assisted printing is compatible with a series of viscosities and can print mammalian cells without affecting cell viability or cell function. Cells can be deposited at a density of up to 10⁸ cells/ml with microscale resolution of a single cell per drop.

### Electrospinning:

Electrospinning is a fiber production technique, which uses electric force to draw charged threads of polymer solutions, or polymer melts.

According to some of any of the embodiments described herein, the additive manufacturing (bioprinting) is or comprises digital light processing (DLP), as described herein.

According to an aspect of some embodiments of the present invention, there is provided a process (or method) of additive manufacturing using DLP technology, wherein a three-dimensional object featuring, in at least a portion thereof, a biological or a biocompatible material is prepared. In some embodiments, a 3D digital model is created or obtained through the use of computer-aided design (CAD) software, as described herein. In an embodiment, specialized slicing software is employed, slicing the 3D model into distinct cross-sectional layers, or “slices” as described herein, with varying layer thickness and slicing parameters. In accordance with some embodiments, a curable formulation is chosen with specific regard to the desired properties and characteristics of the final object, and the vat is prepared. In some embodiments, the DLP 3D printing process is implemented by following the method steps as described herein in any of the respective embodiments. In an embodiment, the digital light projector, through the projection of highly detailed images onto the curable formulation surface, ensures precision and accuracy in layer-by-layer additive printing. According to some embodiments, post-processing procedures are carried out after the completion of printing, including rinsing to remove excess, uncured formulation and optional post-curing (e.g., by application of electromagnetic irradiation and/or heat).

According to some of any of the embodiments described herein, the additive manufacturing is effected by sequentially exposing in a layer-wise manner a curable formulation as described herein in any of the respective embodiments and any combination thereof to a curing condition such as UV irradiation, as described herein in any of the respective embodiments and any combination thereof, and the exposing is performed in a configured pattern corresponding to the shape of the object (in accordance with a pre-determined computerized software as described herein), such that the curable formulation is hardened in each layer at locations exposed to the curing condition, as typically performed in DLP processes. In some of any of these embodiments, the thickness of each layer, the level of irradiation (energy dose) and/or the time of exposing each layer to irradiation are determined or manipulated so as to provide the optimal resolution for a curable formulation of choice. According to some of any of the embodiments described herein, the level of irradiation and the time of exposing to irradiation is as described herein in any of the respective embodiments and any combination thereof.

### The Object:

Herein throughout, in the context of bioprinting, the term “object” describes a final product of the additive manufacturing which comprises, in at least a portion thereof, a biological component. This term refers to the product obtained by a bioprinting method as described herein, after removal of the support material, if such has been used as part of the uncured building material.

The term “object” as used herein throughout refers to a whole object or a part thereof.

In the context of the present embodiments, the object comprises in at least a portion thereof a biocompatible material obtained from the biocompatible photocurable material(s) as described herein, for example, a collagen-based material. In some embodiments, the object is in a form of a scaffold, for example, a hydrogel scaffold, as described herein.

By “collagen-based material” it is meant a material that comprises collagen, preferably a (e.g., recombinant) human collagen as described herein in any of the respective embodiments and any combination thereof.

In some of any of the embodiments described herein, the collagen-based material comprises a scaffold, for example, a hydrogel scaffold, made of a three-dimensional fibrillar network that comprises collagen (e.g., recombinant human collagen) as described herein.

The three-dimension network or scaffold can be in a form of, for example, a film, a sponge, a porous structure, a hydrogel, and any other form, according to a desired need.

In some of any of the embodiments described herein, the object is in a form of a tissue or organ. Such an object can be formulated in accordance with a respective 3D printing data of a desired organ or tissue, using, in addition to the curable collagen as described herein, additional curable materials and biological materials as described herein.

In some embodiments, the object is an implantable object. In some embodiments, the object is an artificial skin. In some embodiments, the object is an artificial tissue (e.g., connective tissue, or muscle tissue such as cardiac tissue and pancreatic tissue). Examples of connective tissues include, but are not limited to, cartilage (including, elastic, hyaline, and fibrocartilage), adipose tissue, reticular connective tissue, embryonic connective tissues (including mesenchymal connective tissue and mucous connective tissue), tendons, ligaments, and bone.

In some embodiments, the object is usable in, or is for use in, constructing an artificial organ or tissue.

The object can comprise hardened materials formed of the one or more photocurable curable materials as described herein in any of the respective embodiments, biological components or materials, as described herein in any of the respective embodiments, and/or non-curable materials as described herein in any of the respective embodiments.

The scaffolds may be administered to subjects in need thereof for the regeneration of tissue such as connective tissue, muscle tissue such as cardiac tissue and pancreatic tissue, or any other tissue, or can be used for research purposes.

The films can be used to construct biomedical devices such as, for example, collagen membranes for hemodialysis.

According to some embodiments, films or scaffolds can be used in cell cultures.

The phrase “cell culture” or “culture” as used herein refers to the maintenance of cells in an artificial, e.g., an in vitro environment. It is to be understood, however, that the term “cell culture” is a generic term and may be used to encompass the cultivation not only of individual prokaryotic (e.g., bacterial) or eukaryotic (e.g., animal, plant and fungal) cells, but also of tissues, organs, organ systems or whole organisms.

In some embodiments, the films or scaffolds can be used in a wound healing process.

The object of the present embodiments comprises a myriad of other uses including, but not limited to, in the treatment of diseases such as interstitial cystitis, scleroderma, and rheumatoid arthritis cosmetic surgery, as a healing aid for burn patients, as a wound-healing agent, as a dermal filler, for spinal fusion procedures, for urethral bulking, in duraplasty procedures, for reconstruction of bone and a wide variety of dental, orthopedic and surgical purposes.

The object may form a part of an article-of-manufacturing such as, for example, a medical device, including an implantable medical device (e.g., a breast implant or a dermal filler).

### Kits:

According to an aspect of some embodiments of the present invention, there is provided a kit that comprises a curable formulation as described herein in any of the respective embodiments.

According to some of any of the embodiments described herein, the kit is identified for use, or is usable, as a modeling material formulation for additive manufacturing (e.g., bioprinting) of an object as described herein in any of the respective embodiments.

According to some of any of the embodiments described herein, the kit further comprises an aqueous carrier, as described herein in any of the respective embodiments. In some embodiments, the bioink composition and the aqueous carrier are packaged individually within the kit.

Alternatively, the kit includes instructions to prepare a modeling material formulation as described herein, by mixing the bioink composition with the aqueous carrier.

The kit may further comprise other components that can be included in a bioink composition or a modeling material formulation(s) as described herein in any of the respective embodiments and any combination thereof.

The kit may further comprise instructions how to use the bioink composition or formulation in an additive manufacturing process as described herein.

The following describes exemplary, non-limiting, embodiments of the present invention.

Embodiment 1: In some embodiments of this invention, a curable formulation suitable for additive manufacturing, such as Digital Light Processing (DLP), of three-dimensional objects is presented. The formulation includes a photoinitiator, a photocurable biological or biocompatible material, and a dye substance capable of absorbing light in the range of 300 nm to 800 nm. This dye substance contains minocycline.

Embodiment 2: In certain embodiments, the curable formulation for additive manufacturing, including the components as described in Embodiment 1, also incorporates minocycline. The amount of minocycline in this embodiment ranges from about 0.01 to about 5% by weight of the total formulation.

Embodiment 3: In various embodiments, a curable formulation suitable for additive manufacturing, like DLP, of three-dimensional objects comprises a photoinitiator, a photocurable biological or biocompatible material, and a dye substance capable of absorbing light within the 300 nm to 800 nm range. The dye substance includes a compound featuring a tetracycline skeleton.

Embodiment 4: In accordance with certain embodiments, the curable formulation described in Embodiment 3 contains a compound featuring a tetracycline skeleton. In this embodiment, the amount of the tetracycline compound ranges from about 0.01 to about 5% by weight of the total formulation.

Embodiment 5: Within these embodiments, the compound featuring a tetracycline skeleton, as described in Embodiment 3, can be represented by a chemical structure similar to Formula X, as defined in the claims. Formula X exhibits specific characteristics, including [Y, R1, R2-R5, R6-R9 definitions] as per the claims.

Embodiment 6: In specific embodiments, the tetracycline compound, as mentioned in Embodiment 3 or 4, may have Y as amine.

Embodiment 7: In various embodiments consistent with Embodiment 5 or 6, Y within the tetracycline compound may be amine.

Embodiment 8: In some embodiments described herein, R2 and R3 within the tetracycline compound, as per Embodiments 3 or 4, are each hydrogen.

Embodiment 9: In certain embodiments, the compound featuring a tetracycline skeleton, such as those described in Embodiments 3 or 4, can include tetracycline and its various derivatives, such as dihydrosteffimycin, demethyltetracycline, and others as listed in the claims.

Embodiment 10: In some embodiments, the curable formulation, which includes a compound featuring a tetracycline skeleton as outlined in Embodiments 3 or 4, may comprise tetracycline derivatives like oxytetracycline, lymcycline, chlorotetracycline, and others, as mentioned in the claims.

Embodiment 11: In accordance with these embodiments, the curable formulation, which includes a compound featuring a tetracycline skeleton as described in Embodiments 3 or 4, may include tetracycline derivatives like minocycline, doxycycline, tetracycline, or their pharmaceutically acceptable salts, as specified in the claims.

Embodiment 12: In some embodiments, the salt of minocycline in the curable formulation can be minocycline hydrochloride.

Embodiment 13: In certain embodiments, the salt of tetracycline in the curable formulation can be tetracycline hydrochloride.

Embodiment 14: In various embodiments, the salt of doxycycline in the curable formulation can be doxycycline hyclate.

Embodiment 15: In some embodiments, the curable formulation may include an aqueous carrier.

Embodiment 16: In certain embodiments, the aqueous solution in the curable formulation may be selected from a neutral aqueous solution or an acidic aqueous solution.

Embodiment 17: In some embodiments, the amount of the photoinitiator in the curable formulation may range from 0.01 to 1% by weight of the total formulation.

Embodiment 18: In certain embodiments, the amount of the photoinitiator in the curable formulation may range from 0.1 to 1% by weight of the total formulation.

Embodiment 19: In various embodiments, the amount of the photoinitiator in the curable formulation may range from 0.01 to 5% by weight of the total formulation.

Embodiment 20: In some embodiments, the amount of the photoinitiator in the curable formulation may range from 0.01 to 1% by weight of the total formulation.

Embodiment 21: In certain embodiments, the photocurable biocompatible material in the curable formulation may comprise a collagen that features a plurality of photocurable groups.

Embodiment 22: In some embodiments, the photocurable groups in the curable formulation may comprise (meth)acrylic groups.

Embodiment 23: In various embodiments, the collagen in the curable formulation may be human Type I collagen.

Embodiment 24: In some embodiments, the collagen in the curable formulation may be a recombinant collagen.

Embodiment 25: In certain embodiments, the collagen in the curable formulation may be a plant-derived recombinant collagen.

Embodiment 26: In some embodiments, the plant-derived recombinant collagen in the curable formulation may be human Type I collagen.

Embodiment 27: In certain embodiments, the curable formulation may further comprise an additional curable material that features a plurality of photocurable groups.

Embodiment 28: In some embodiments, a process of additive manufacturing a three-dimensional object may be provided. The process includes sequentially exposing, in a layerwise manner, a modeling material formulation to a curing condition suitable for hardening the curable formulation. This exposing is in a configured pattern corresponding to the shape of the object.

Embodiment 29: In certain embodiments of the above process, the exposing is for a time period that ranges from 1 second to 120 seconds for each layer.

Embodiment 30: In some embodiments, the curing condition in the process may comprise irradiation.

Embodiment 31: In certain embodiments, the irradiation in the process may be at a wavelength within the UV-vis range.

Embodiment 32: In some embodiments, the irradiation in the process may be at a wavelength that ranges from 300 to 800 nm, or from 300 to 600 nm, or from 300 to 500 nm, or from 350 to 450 nm.

Embodiment 33: In certain embodiments of the above process, the irradiation is at a level that ranges from 1 to 150 mW/cm².

Embodiment 34: In some embodiments, the additive manufacturing process may be implemented using DLP technology.

Embodiment 35: In certain embodiments, a three-dimensional object obtained by the above process may be provided.

Embodiment 36: In some embodiments, a three-dimensional object may be described, comprising, in at least a portion thereof, a hardened curable formulation as defined in any one of the above claims.

As used herein the term “about” refers to ±10% or ±5%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

Herein throughout, whenever “centipoise” or “Cp” is indicated, the corresponding Pa·second value (1 Pa·second=1,000 centipoise) is encompassed.

Herein throughout, whenever the phrase “weight percent”, or “% by weight” or “% wt.”, is indicated in the context of embodiments of a formulation (e.g., a modeling formulation, a curable formulation, a bioink composition), it is meant weight percent of the total weight of the respective uncured formulation.

Herein throughout, the term “water-miscible” describes a material which is at least partially dissolvable or dispersible in water, that is, at least 50% of the molecules move into the water upon mixture. This term encompasses the terms “water-soluble” and “water dispersible”.

Herein throughout, the term “water-soluble” describes a material that when mixed with water in equal volumes or weights, a homogeneous solution is formed.

Herein throughout, the phrase “linking moiety” or “linking group” describes a group that connects two or more moieties or groups in a compound. A linking moiety is typically derived from a bi- or tri-functional compound, and can be regarded as a bi- or tri-radical moiety, which is connected to two or three other moieties, via two or three atoms thereof, respectively.

Exemplary linking moieties include a hydrocarbon moiety or chain, optionally interrupted by one or more heteroatoms, as defined herein, and/or any of the chemical groups listed below, when defined as linking groups.

When a chemical group is referred to herein as “end group” it is to be interpreted as a substituent, which is connected to another group via one atom thereof.

Herein throughout, the term “hydrocarbon” collectively describes a chemical group composed mainly of carbon and hydrogen atoms. A hydrocarbon can be comprised of alkyl, alkene, alkyne, aryl, and/or cycloalkyl, each can be substituted or unsubstituted, and can be interrupted by one or more heteroatoms. The number of carbon atoms can range from 2 to 30, and is preferably lower, e.g., from 1 to 10, or from 1 to 6, or from 1 to 4. A hydrocarbon can be a linking group or an end group.

As used herein, the term “amine” describes both a —NR′R″ group and a —NR′— group, wherein R′ and R″ are each independently hydrogen, alkyl, cycloalkyl, aryl, as these terms are defined hereinbelow.

The amine group can therefore be a primary amine, where both R′ and R″ are hydrogen, a secondary amine, where R′ is hydrogen and R″ is alkyl, cycloalkyl or aryl, or a tertiary amine, where each of R′ and R″ is independently alkyl, cycloalkyl or aryl.

Alternatively, R′ and R″ can each independently be hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, carbonyl, C-carboxylate, O-carboxylate, N-thiocarbamate, O-thiocarbamate, urea, thiourea, N-carbamate, O-carbamate, C-amide, N-amide, guanyl, guanidine and hydrazine.

The term “amine” is used herein to describe a —NR′R″ group in cases where the amine is an end group, as defined hereinunder, and is used herein to describe a —NR′— group in cases where the amine is a linking group or is or part of a linking moiety.

The term “alkyl” describes a saturated aliphatic hydrocarbon including straight chain and branched chain groups. Preferably, the alkyl group has 1 to 30, or 1 to 20 carbon atoms. Whenever a numerical range; e.g., “1-20”, is stated herein, it implies that the group, in this case the alkyl group, may contain 1 carbon atom, 2 carbon atoms, 3 carbon atoms, etc., up to and including 20 carbon atoms. The alkyl group may be substituted or unsubstituted. Substituted alkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine.

The alkyl group can be an end group, as this phrase is defined hereinabove, wherein it is attached to a single adjacent atom, or a linking group, as this phrase is defined hereinabove, which connects two or more moieties via at least two carbons in its chain. When the alkyl is a linking group, it is also referred to herein as “alkylene” or “alkylene chain”.

Alkene and Alkyne, as used herein, are an alkyl, as defined herein, which contains one or more double bond or triple bond, respectively.

The term “cycloalkyl” describes an all-carbon monocyclic ring or fused rings (i.e., rings which share an adjacent pair of carbon atoms) group where one or more of the rings does not have a completely conjugated pi-electron system. Examples include, without limitation, cyclohexane, adamantine, norbornyl, isobornyl, and the like. The cycloalkyl group may be substituted or unsubstituted. Substituted cycloalkyl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The cycloalkyl group can be an end group, as this phrase is defined hereinabove, wherein it is attached to a single adjacent atom, or a linking group, as this phrase is defined hereinabove, connecting two or more moieties at two or more positions thereof.

The term “heteroalicyclic” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofurane, tetrahydropyrane, morpholino, oxalidine, and the like.

The heteroalicyclic may be substituted or unsubstituted. Substituted heteroalicyclic may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroalicyclic group can be an end group, as this phrase is defined hereinabove, where it is attached to a single adjacent atom, or a linking group, as this phrase is defined hereinabove, connecting two or more moieties at two or more positions thereof.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted. Substituted aryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, N-carbamate, O-carbamate, C-amide, N-amide, guanyl, guanidine and hydrazine. The aryl group can be an end group, as this term is defined hereinabove, wherein it is attached to a single adjacent atom, or a linking group, as this term is defined hereinabove, connecting two or more moieties at two or more positions thereof.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. Examples, without limitation, of heteroaryl groups include pyrrole, furan, thiophene, imidazole, oxazole, thiazole, pyrazole, pyridine, pyrimidine, quinoline, isoquinoline and purine. The heteroaryl group may be substituted or unsubstituted. Substituted heteroaryl may have one or more substituents, whereby each substituent group can independently be, for example, hydroxyalkyl, trihaloalkyl, cycloalkyl, alkenyl, alkynyl, aryl, heteroaryl, heteroalicyclic, amine, halide, sulfonate, sulfoxide, phosphonate, hydroxy, alkoxy, aryloxy, thiohydroxy, thioalkoxy, thioaryloxy, cyano, nitro, azo, sulfonamide, C-carboxylate, O-carboxylate, N-thiocarbamate, O-thiocarbamate, urea, thiourea, O-carbamate, N-carbamate, C-amide, N-amide, guanyl, guanidine and hydrazine. The heteroaryl group can be an end group, as this phrase is defined hereinabove, where it is attached to a single adjacent atom, or a linking group, as this phrase is defined hereinabove, connecting two or more moieties at two or more positions thereof. Representative examples are pyridine, pyrrole, oxazole, indole, purine and the like.

The term “halide” and “halo” describes fluorine, chlorine, bromine or iodine.

The term “haloalkyl” describes an alkyl group as defined above, further substituted by one or more halide.

The term “carbonyl” or “carbonate” as used herein, describes a —C(═O)—R′ end group or a —C(═O)— linking group, as these phrases are defined hereinabove, with R′ as defined herein.

The term “thiocarbonyl” as used herein, describes a —C(═S)—R′ end group or a —C(═S)— linking group, as these phrases are defined hereinabove, with R′ as defined herein.

The term “oxo” as used herein, describes a (═O) group, wherein an oxygen atom is linked by a double bond to the atom (e.g., carbon atom) at the indicated position.

The term “thiooxo” as used herein, describes a (═S) group, wherein a sulfur atom is linked by a double bond to the atom (e.g., carbon atom) at the indicated position.

The term “oxime” describes a ═N—OH end group or a ═N—O— linking group, as these phrases are defined hereinabove.

The term “hydroxyl” describes a —OH group.

The term “alkoxy” describes both an —O-alkyl and an —O-cycloalkyl group, as defined herein. The term alkoxide describes —R′O⁻ group, with R′ as defined herein.

The term “aryloxy” describes both an —O-aryl and an —O-heteroaryl group, as defined herein.

The term “thiohydroxy” or “thiol” describes a —SH group. The term “thiolate” describes a —S⁻ group.

The term “thioalkoxy” describes both a —S-alkyl group, and a —S-cycloalkyl group, as defined herein.

The term “thioaryloxy” describes both a —S-aryl and a —S-heteroaryl group, as defined herein.

The “hydroxyalkyl” is also referred to herein as “alcohol”, and describes an alkyl, as defined herein, substituted by a hydroxy group.

The term “acyl halide” describes a —(C═O)R″″ group wherein R″″ is halide, as defined hereinabove.

The term “carboxylate” as used herein encompasses C-carboxylate and O-carboxylate.

The term “C-carboxylate” describes a —C(═O)—OR′ end group or a —C(═O)—O— linking group, as these phrases are defined hereinabove, where R′ is as defined herein.

The term “O-carboxylate” describes a —OC(═O)R′ end group or a —OC(═O)— linking group, as these phrases are defined hereinabove, where R′ is as defined herein.

A carboxylate can be linear or cyclic. When cyclic, R′ and the carbon atom are linked together to form a ring, in C-carboxylate, and this group is also referred to as lactone. Alternatively, R′ and O are linked together to form a ring in O-carboxylate. Cyclic carboxylates can function as a linking group, for example, when an atom in the formed ring is linked to another group.

The term “thiocarboxylate” as used herein encompasses C-thiocarboxylate and O-thiocarboxylate.

The term “C-thiocarboxylate” describes a —C(═S)—OR′ end group or a —C(═S)—O— linking group, as these phrases are defined hereinabove, where R′ is as defined herein.

The term “O-thiocarboxylate” describes a —OC(═S) R′ end group or a —OC(═S)— linking group, as these phrases are defined hereinabove, where R′ is as defined herein.

A thiocarboxylate can be linear or cyclic. When cyclic, R′ and the carbon atom are linked together to form a ring, in C-thiocarboxylate, and this group is also referred to as thiolactone. Alternatively, R′ and O are linked together to form a ring in O-thiocarboxylate. Cyclic thiocarboxylates can function as a linking group, for example, when an atom in the formed ring is linked to another group.

The term “carbamate” as used herein encompasses N-carbamate and O-carbamate.

The term “N-carbamate” describes an R″OC(═O)—NR′—end group or a —OC(═O)—NR′— linking group, as these phrases are defined hereinabove, with R′ and R″ as defined herein.

The term “O-carbamate” describes an —OC(═O)—NR′R″ end group or an —OC(═O)— NR′— linking group, as these phrases are defined hereinabove, with R′ and R″ as defined herein.

A carbamate can be linear or cyclic. When cyclic, R′ and the carbon atom are linked together to form a ring, in O-carbamate. Alternatively, R′ and O are linked together to form a ring in N-carbamate. Cyclic carbamates can function as a linking group, for example, when an atom in the formed ring is linked to another group.

The term “thiocarbamate” as used herein encompasses N-thiocarbamate and O-thiocarbamate.

The term “O-thiocarbamate” describes a —OC(═S)—NR′R″ end group or a —OC(═S)—NR′— linking group, as these phrases are defined hereinabove, with R′ and R″ as defined herein.

The term “N-thiocarbamate” describes an R″OC(═S)NR′— end group or a —OC(═S)NR′— linking group, as these phrases are defined hereinabove, with R′ and R″ as defined herein.

Thiocarbamates can be linear or cyclic, as described herein for carbamates.

The term “dithiocarbamate” as used herein encompasses S-dithiocarbamate and N-dithiocarbamate.

The term “S-dithiocarbamate” describes a —SC(═S)—NR′R″ end group or a —SC(═S)NR′— linking group, as these phrases are defined hereinabove, with R′ and R″ as defined herein.

The term “N-dithiocarbamate” describes an R″SC(═S)NR′— end group or a —SC(═S)NR′— linking group, as these phrases are defined hereinabove, with R′ and R″ as defined herein.

The term “urea”, which is also referred to herein as “ureido”, describes a —NR′C(═O)—NR″R′″ end group or a —NR′C(═O)—NR″— linking group, as these phrases are defined hereinabove, where R′ and R″ are as defined herein and R′″ is as defined herein for R′ and R″.

The term “thiourea”, which is also referred to herein as “thioureido”, describes a —NR′—C(═S)—NR″R′″ end group or a —NR′—C(═S)—NR″— linking group, with R′, R″ and R′″ as defined herein.

The term “amide” as used herein encompasses C-amide and N-amide.

The term “C-amide” describes a —C(═O)—NR′R″ end group or a —C(═O)—NR′— linking group, as these phrases are defined hereinabove, where R′ and R″ are as defined herein.

The term “N-amide” describes a R′C(═O)—NR″—end group or a R′C(═O)—N— linking group, as these phrases are defined hereinabove, where R′ and R″ are as defined herein.

An amide can be linear or cyclic. When cyclic, R′ and the carbon atom are linked together to form a ring, in C-amide, and this group is also referred to as lactam. Cyclic amides can function as a linking group, for example, when an atom in the formed ring is linked to another group.

As used herein, the term “alkylene glycol” describes a —O—[(CR′R″)_{z}—O]_{y}—R′″ end group or a —O—[(CR′R″)_{z}—O]_{y}— linking group, with R′, R″ and R′″ being as defined herein, and with z being an integer of from 1 to 10, preferably, from 2 to 6, more preferably 2 or 3, and y being an integer of 1 or more. Preferably R′ and R″ are both hydrogen. When z is 2 and y is 1, this group is ethylene glycol. When z is 3 and y is 1, this group is propylene glycol. When y is 2-4, the alkylene glycol is referred to herein as oligo (alkylene glycol). When y is higher than 4, it is a poly(alkylene glycol). Capped poly(alkylene glycol) has R′″ which is other than hydrogen and which can be, for example, an alkyl (e.g., lower alkyl), a carbonyl, and like moieties.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Materials and Experimental Methods

### Materials

Minocycline (as HCl salt) was obtained from Apollo Scientific; BIM0172LOT: AS472664.

Doxycycline (as hyclate salt) was obtained from Sigma Aldrich; D5207

Tetracycline (as HCl salt) was obtained from Sigma Aldrich; T7660-5g

Lithium phenyl-2,4,6-trimethylbenzoylphosphinate (LAP) was obtained from Bezwada Biomedical; BB-0308-82

Sodium phenyl-2,4,6-trimethylbenzoylphosphinate (NAP) was obtained from Bezwada Biomedical; BBO309

Poly(ethylene glycol) diacrylate (PEG-DA), varying in average MW (e.g., between 1,000 and 10,000 or between 3,000 and 6,000 kDa,) were obtained from Bezwada Biomedical). HCl 10 mM was prepared from 1M HCl.

CMR describes curable rhCollagen featuring a plurality of methacrylate groups conjugated to the lysine residues of the rhCollagen, as described in WO 2018/225076. CMR50 relates to CMR in which 50% of the lysine residues have methacrylate groups conjugates thereto.

UV absorbance was measured with spectrophotometer E-0037.

Kinetic studies were performed using a Discovery HR-2 Rheometer [TA instruments] equipped with Plate-Plate (20 mm, quartz plate) geometry, with sample conditioning at 22 °C, pre-shear in 1.0 rad/sec for 10 sec. Following that, Oscillation Fast Sampling test was applied, with 10% strain at 2 Hz, 30 sec delay followed by 50 mW/cm² irradiation for 7.5 sec.

### Example 1

The present inventors have conceived using minocycline as a photoabsorber in 3D-bioprinting, and have studied its performance in DLP 3D-bioprinting processes that employ collagen-containing curable formulations such as described in WO 2018/225076 and co-filed PCT International Patent Application No. PCT/IL2022/051143 (published as WO 2023/073711).

In an exemplary study, a curable formulation that comprises curable rhCollagen featuring a plurality of methacrylate groups, which is also referred to herein as CMR, an aqueous carrier, one or more additional curable polymeric materials (e.g., PEG-DA), and a photoinitiator, such as described in WO 2018/225076, was used.

In an exemplary procedure, minocycline (HCl salt), at varying final concentrations, was dissolved in mQ water, along with Lithium phenyl-2,4,6-trimethylbenzoylphosphinate (LAP, a photoinitiator). PEG (6000)-diacrylate was added to the solution, followed by the addition of CMR50 (at a final concentration of 15.8 mg/mL) and diluted HCl (10 mM). The resulting mixtures were each stirred until a homogenous solution was obtained, and were used to 3D print (using a DLP printer) a hydrogel featuring 200 μm channels along the Z axis and in the XY plane.

The effect of the concentration of minocycline on the hardening rate and degree of the formulation was tested, by determining the G′ values of each formulation during the curing step.

Measurements were performed using a Discovery HR-2 rheometer equipped with a 20 mm parallel plate geometry and an Omnicure (series 2000) optics attachment as the light source. 25-90 μL of each sample were loaded onto the bottom plate and the top geometry was lowered to obtain a gap size of 50-250 μm, respective to the drop volume. Measurement's duration was set to 120 seconds with angular frequency of 2 Hz and a 1% strain in which the sample was preconditioned for 30 seconds and then light was initiated, using an external UV 365 nm light source, for 60 seconds at 50 mW/cm².

Formulations containing 0, 0.04, 0.08, 0.12, 0.16 and 0.2, % by weight minocycline were tested and the results are presented in FIG. 1.

As can be seen, all the formulations have reached the maximum crosslinking level, indicating that at the high light energy used, the minocycline does not affect the crosslinking of the formulation, also at a concentration of 0.08% by weight. Thus, all formulations reached the maximum crosslinking level, and the time to G′ increased when the concentration of minocycline was increased, as observed even in minute concentrations.

### Example 2

Minocycline and tetracycline analogs thereof, tetracycline as HCl salt and doxycycline as hyclate salt, were further studied for their compatibility and suitability with the additive manufacturing (3D bioprinting) process requirements, in terms of solubility, UV absorbance, hardening kinetics, and 3D Printing resolution, in accordance with the following procedures.

### Solubility:

5 mg/ml of a tested photoabsorber were added to a 10 mM HCl solution at room temperature and the solution was stirred. Solubility was assessed by visual inspection after 5 minutes. A clear solution and lack of precipitation are indicative that the photoabsorber is soluble in 10 mM HCl. All three tested photoabsorbers were found to be fully soluble in 10 mM HCl at room temperature.

### UV Absorbance in HCl Solution:

Photoabsorbers were tested at the same concentration (e.g., 0.5 w/w %) which equals to about 10 mM PB in HCL solution, as follows:
0.5% minocycline HCl (MW=493.94 grams/mol) are 10 mM;
0.5% doxycycline hyclate (MW=512.94 grams/mol) are 9.74 mM;
0.5% tetracycline HCl (MW=480.90 grams/mol) are 10.39 mM.

Solubility in 10 mM HCl was confirmed and solutions were diluted if necessary.

A 10 mM HCL solution was used as a reference sample; and a 10 mM (or 0.5% by weight) solution of each of the tested photoabsorbers in 1.5 mL of 10 mM HCl were prepared as tested samples.

UV absorbance was measured with spectrophotometer E-0037, upon transferring 1 mL of each solution to a disposable cuvette. Results were evaluated using an absorbance-intensity curve that was plotted as a function of wavelength. Amax of the photoabsorber was extracted from the curve as well as the absorbance intensity at 385 and 405 nm.

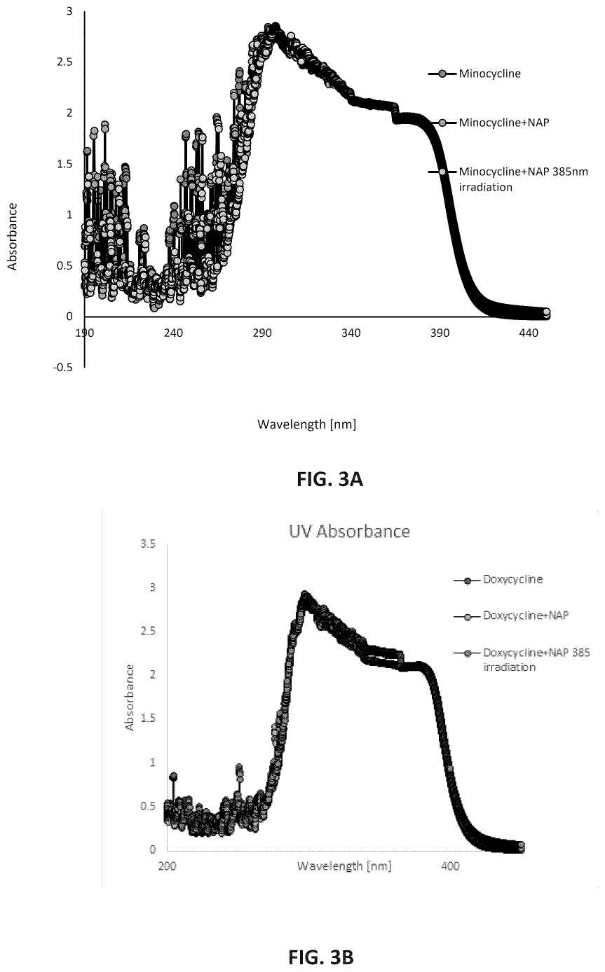

FIG. 2 presents the absorbance data of minocycline HCl.

### UV Absorbance in the Presence of a Photoinitiator:

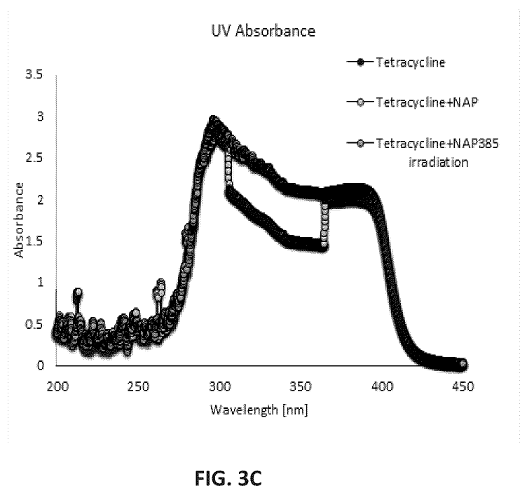

UV absorbance measurements were performed for 1 mL 10 mM of a HCl solution containing each tested photoabsorber at a concentration of 10 Mm alone, or in combination with 0.5% by weight NAP (from a 25 mg/mL stock solution in DDW), before and after irradiation at 385 nm at 50-100% intensity. The obtained data is presented in FIGS. 3A-C, and show a substantially identical UV absorbance pattern each tested photoabsorber without NAP or combined with NAP before and after irradiation.

### Kinetic Evaluation:

The hardening (e.g., as a result of polymerization and/or cross-linking of the CMR) kinetics of different formulations were measured using Discovery HR-2 rheometer equipped with a 20 mm parallel plate geometry and an Omnicure (series 2000) optics attachment as the light source. 25-90 μL of each sample were loaded onto the bottom plate and the top geometry was lowered to obtain a gap size of 50-250 μm, respective to the drop volume. Measurement's duration was set to 120 seconds with angular frequency of 2 Hz and a 1% strain in which the sample was preconditioned for 30 seconds and then light was initiated, using an external UV 365 nm light source, for 60 seconds at 50 mW/cm².

Each of the tested formulations included 10 mM HCl, 0.5% by weight CMR, 0.5% by weight NAP and 0.08% or 0.2% by weight of the tested tetracycline compound. Control formulations were devoid of the tetracycline compound.

The obtained data is presented in FIGS. 4A-C and show that for minocycline and doxycycline an increase in PB concentration affects the curing kinetics of the formulation without adversely affecting the final storage modulus G′ of the hardened formulation. For Tetracycline the increase in the PB concentration affects both the curing kinetics and the storage modulus G′.

### 3D Printing Resolution:

Resolution models were printed with B9Creations “6 series Mpro 80 micron” 3D printer, with layer thickness of 150 micrometers and exposure of 120 mJ/cm². It is to be noted that these parameters can be adjusted so as to provide optimal resolution for a given concentration of the photoinitiator and the photoabsorber.

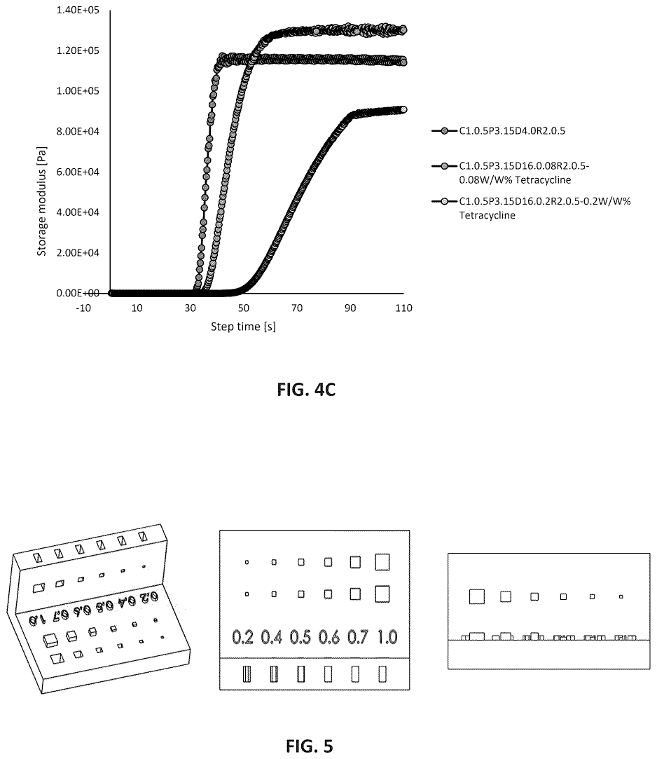

An isometric view of the resolution model is presented in FIG. 5 (left), the XY plane resolution in FIG. 5 (middle) and the Z axis resolution in FIG. 5 (right).

All tested formulations included 0.5% by weight NAP and 0.2% by weight of the tested tetracycline compound, in addition to 10 mM HCl, 0.5% by weight CMR, PRG-DA and water.

Photographs of the printed models are presented in FIGS. 6A-C, and show that all tested tetracycline compounds resulted in high resolution models as apparent from open pores visible in the Z axis. Under the specific parameters of this test, the results achieved with tetracycline were slightly better, however, exposure can be adjusted to achieve comparable results with the other tested compounds.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

What is claimed is:

1-2. (canceled)

3. A curable formulation for use in additive manufacturing of a three-dimensional object, the formulation comprising a photoinitiator, a collagen that features a plurality of photocurable groups and a dye substance capable of absorbing light at a wavelength of from 300 nm to 800 nm (a photoabsorber), said dye substance comprising a compound featuring a tetracycline skeleton.

4. The curable formulation of claim 3, wherein an amount of said compound featuring a tetracycline skeleton ranges from 0.01 to 5% by weight of the total weight of the formulation.

5. The curable formulation of claim 3, wherein said compound featuring a tetracycline skeleton is represented by Formula X: 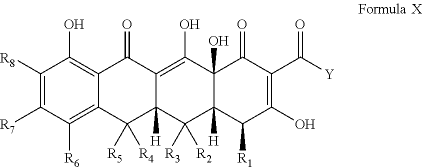
or a pharmaceutically acceptable salt thereof,
wherein:
Y is selected from amine, hydroxy, alkoxy, alkyl, O—NR′R″, thiol, thioalkoxy, thioaryloxy, aryl, cycloalkyl and halo;
R₁ is selected from hydrogen, amine, alkyl, cycloalkyl, aminoalkyl, aryl, hydroxy, alkoxy, aryloxy, thiol, thioalkoxy, thioaryloxy, halo, carboxy, amide, aminoether, carbamate, oxo, and like substituents; and
R₂-R₅ are each independently selected from hydrogen, amine, alkyl, cycloalkyl, aminoalkyl, aminoether, aryl, hydroxy, alkoxy, aryloxy, thiol, thioalkoxy, thioaryloxy, halo, carboxy, amide, carbamate, oxo, and like substituents, or, alternatively, R₂ and R₃ and/or R₄ and R₅ form together an alkene, oxo or an alicyclic or heteroalicyclic ring; and
R₆-R₉ are each independently selected from hydrogen, amine, alkyl, cycloalkyl, aminoalkyl, aminoether, aryl, hydroxy, alkoxy, aryloxy, thiol, thioalkoxy, thioaryloxy, halo, carboxy, amide, carbamate, oxo, and like substituents, or, alternatively, R₆ and R₇ or R₇ and R₈ form together an alicyclic or heteroalicyclic ring.

6. The curable formulation of claim 5, wherein R₁ is amine.

7. The curable formulation of claim 5, wherein Y is amine.

8. The curable formulation of claim 5, wherein R₂ and R₃ are each hydrogen.

9. The curable formulation of claim 3, wherein said compound featuring a tetracycline skeleton is selected from dihydrosteffimycin, demethyltetracycline, aclacinomycin, akrobomycin, baumycin, bromotetracycline, cetocyclin, chlortetracycline, clomocycline, daunorubicin, demeclocycline, doxorubicin, doxorubicin hydrochloride, doxycycline, lymecyclin, marcellomycin, meclocycline, meclocycline sulfosalicylate, methacycline, minocycline, minocycline hydrochloride, musettamycin, oxytetracycline, rhodirubin, rolitetracycline, rubomycin, serirubicin, steffimycin, tetracycline, and a pharmaceutically acceptable salt of any one of the foregoing.

10. The curable formulation of claim 3, wherein said compound featuring a tetracycline skeleton is selected from oxytetracycline, lymcycline, chlorotetracycline, oxytetarcycline, demeclocycline, meclocycline, methacycline, rolitetracycline, tigecycline, eravacycline, sarecycline and omadacycline and a pharmaceutically acceptable salt of any one of the foregoing.

11. The curable formulation of claim 3, wherein said compound featuring a tetracycline skeleton is selected from minocycline, doxycycline, tetracycline and a pharmaceutically acceptable salt of any one of the foregoing.

12. The curable formulation of claim 11, wherein the salt of minocycline is minocycline hydrochloride, the salt of tetracycline is tetracycline hydrochloride, and the salt of doxycycline is doxycycline hyclate.

13-14. (canceled)

15. The curable formulation of claim 3, wherein an amount of said photoinitiator ranges from 0.01 to 1% by weight of the total weight of the formulation.

16-17. (canceled)

18. The curable formulation of claim 3, wherein an amount of said compound featuring a tetracycline skeleton dye substance-ranges from 0.01 to 1% by weight of the total weight of the formulation.

19. (canceled)

20. The curable formulation of claim 3, wherein said photocurable groups comprise (meth)acrylic groups.

21-23. (canceled)

24. The curable formulation of claim 3, wherein the collagen is a plant-derived recombinant human Type I collagen.

25. The curable formulation of claim 3 claim 3, further comprising an additional curable material that features a plurality of photocurable groups.

26-27. (canceled)

28. A process of additive manufacturing a three-dimensional object featuring, in at least a portion thereof, a biological or a biocompatible material, the process comprising sequentially exposing, in a layerwise manner, a modeling material formulation to a curing condition suitable for hardening the curable formulation, said exposing being in a configured pattern corresponding to the shape of the object,
wherein said modeling material formulation comprises the curable formulation of claim 3,
thereby manufacturing the three-dimensional object.

29. The process of claim 28, wherein said exposing is for time period that ranges from 1 second to 120 second for each layer.

30. The process of claim 28, wherein said curing condition comprises irradiation.

31-34. (canceled)

35. A three-dimensional object obtained by:
sequentially exposing, in a layerwise manner, a modeling material formulation to a curing condition suitable for hardening the curable formulation, said exposing being in a configured pattern corresponding to the shape of the object,
wherein said modeling material formulation comprises the curable formulation of claim 3.

36. A three-dimensional object comprising, in at least a portion thereof, a hardened curable formulation as defined in claim 3.

37. (canceled)


SEQUENCE LISTING (16 sequences)

SEQ ID NO: 1 - protein, 1464 aa, Homo sapiens
MFSFVDLRLLLLLAATALLTHGQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPVGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 1366 aa, Homo sapiens [sequence omitted]

SEQ ID NO: 3 - protein, 1464 aa, Homo sapiens; identical to SEQ ID NO: 1

SEQ ID NO: 4 - protein, 1366 aa, Homo sapiens [sequence omitted]

SEQ ID NO: 5 - protein, 1489 aa, Homo sapiens
MAHARVLLLALAVLATAAVAVASSSSFADSNPIRPVTDRAASTLAQLQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGSPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPAGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 6 - protein, 1389 aa, Homo sapiens
MAHARVLLLALAVLATAAVAVASSSSFADSNPIRPVTDRAASTLAQLLQEETVRKGPAGDRGPRGERGPPGPPGRDGEDGPTGPPGPPGPPGPPGLGGNFAAQYDGKGVGLGPGPMGLMGPRGPPGAAGAPGPQGFQGPAGEPGEPGQTGPAGARGPAGPPGKAGEDGHPGKPGRPGERGVVGPQGARGFPGTPGLPGFKGIRGHNGLDGLKGQPGAPGVKGEPGAPGENGTPGQTGARGLPGERGRVGAPGPAGARGSDGSVGPVGPAGPIGSAGPPGFPGAPGPKGEIGAVGNAGPTGPAGPRGEVGLPGLSGPVGPPGNPGANGLTGAKGAAGLPGVAGAPGLPGPRGIPGPVGAAGATGARGLVGEPGPAGSKGESGNKGEPGSAGPQGPPGPSGEEGKRGPNGEAGSAGPPGPPGLRGSPGSRGLPGADGRAGVMGPPGSRGASGPAGVRGPNGDAGRPGEPGLMGPRGLPGSPGNIGPAGKEGPVGLPGIDGRPGPIGPAGARGEPGNIGFPGPKGPTGDPGKNGDKGHAGLAGARGAPGPDGNNGAQGPPGPQGVQGGKGEQGPAGPPGFQGLPGPSGPAGEVGKPGERGLHGEFGLPGPAGPRGERGPPGESGAAGPTGPIGSRGPSGPPGPDGNKGEPGVVGAVGTAGPSGPSGLPGERGAAGIPGGKGEKGEPGLRGEIGNPGRDGARGAHGAVGAPGPAGATGDRGEAGAAGPAGPAGPRGSPGERGEVGPAGPNGFAGPAGAAGQPGAKGERGGKGPKGENGVVGPTGPVGAAGPAGPNGPPGPAGSRGDGGPPGMTGFPGAAGRTGPPGPSGISGPPGPPGPAGKEGLRGPRGDQGPVGRTGEVGAVGPPGFAGEKGPSGEAGTAGPPGTPGPQGLLGAPGILGLPGSRGERGLPGVAGAVGEPGPLGIAGPPGARGPPGAVGSPGVNGAPGEAGRDGNPGNDGPPGRDGQPGHKGERGYPGNIGPVGAAGAPGPHGPVGPAGKHGNRGETGPSGPVGPAGAVGPRGPSGPQGIRGDKGEPGEKGPRGLPGFKGHNGLQGLPGIAGHHGDQGAPGSVGPAGPRGPAGPSGPAGKDGRTGHPGTVGPAGIRGPQGHQGPAGPPGPPGPPGPPGVSGGGYDFGYDGDFYRADQPRSAPSLRPKDYEVDATLKSLNNQIETLLTPEGSRKNPARTCRDLRLSHPEWSSGYYWIDPNQGCTMEAIKVYCDFPTGETCIRAQPENIPAKNWYRSSKDKKHVWLGETINAGSQFEYNVEGVTSKEMATQLAFMRLLANYASQNITYHCKNSIAYMDEETGNLKKAVILQGSNDVELVAEGNSRFTYTVLVDGCSKKTNEWGKTIIEYKTNKPSRLPFLDIAPLDIGGADHEFFVDIGPVCFK

SEQ ID NO: 7 - DNA, 5927 bp, Homo sapiens
  source 1..5927: mol_type = genomic DNA
tcgtcggagcagacgggagtttctcctcggggtcggagcaggaggcacgcggagtgtgaggccacgcatgagcggacgctaaccccctccccagccacaaagagtctacatgtctagggtctagacatgttcagctttgtggacctccggctcctgctcctcttagcggccaccgccctcctgacgcacggccaagaggaaggccaagtcgagggccaagacgaagacatcccaccaatcacctgcgtacagaacggcctcaggtaccatgaccgagacgtgtggaaacccgagccctgccggatctgcgtctgcgacaacggcaaggtgttgtgcgatgacgtgatctgtgacgagaccaagaactgccccggcgccgaagtccccgagggcgagtgctgtcccgtctgccccgacggctcagagtcacccaccgaccaagaaaccaccggcgtcgagggacccaagggagacactggcccccgaggcccaaggggacccgcaggcccccctggccgagatggcatccctggacagcctggacttcccggaccccccggaccccccggacctcccggaccccctggcctcggaggaaactttgctccccagctgtcttatggctatgatgagaaatcaaccggaggaatttccgtgcctggccccatgggtccctctggtcctcgtggtctccctggcccccctggtgcacctggtccccaaggcttccaaggtccccctggtgagcctggcgagcctggagcttcaggtcccatgggtccccgaggtcccccaggtccccctggaaagaatggagatgatggggaagctggaaaacctggtcgtcctggtgagcgtgggcctcctgggcctcagggtgctcgaggattgcccggaacagctggcctccctggaatgaagggacacagaggtttcagtggtttggatggtgccaagggagatgctggtcctgctggtcctaagggtgagcctggcagccctggtgaaaatggagctcctggtcagatgggcccccgtggcctgcctggtgagagaggtcgccctggagcccctggccctgctggtgctcgtggaaatgatggtgctactggtgctgccgggccccctggtcccaccggccccgctggtcctcctggcttccctggtgctgttggtgctaagggtgaagctggtccccaagggccccgaggctctgaaggtccccagggtgtgcgtggtgagcctggcccccctggccctgctggtgctgctggccctgctggaaaccctggtgctgatggacagcctggtgctaaaggtgccaatggtgctcctggtattgctggtgctcctggcttccctggtgcccgaggcccctctggaccccagggccccggcggccctcctggtcccaagggtaacagcggtgaacctggtgctcctggcagcaaaggagacactggtgctaagggagagcctggccctgttggtgttcaaggaccccctggccctgctggagaggaaggaaagcgaggagctcgaggtgaacccggacccactggcctgcccggaccccctggcgagcgtggtggacctggtagccgtggtttccctggcgcagatggtgttgctggtcccaagggtcccgctggtgaacgtggttctcctggccctgctggccccaaaggatctcctggtgaagctggtcgtcccggtgaagctggtctgcctggtgccaagggtctgactggaagccctggcagccctggtcctgatggcaaaactggcccccctggtcccgccggtcaagatggtcgccccggacccccaggcccacctggtgcccgtggtcaggctggtgtgatgggattccctggacctaaaggtgctgctggagagcccggcaaggctggagagcgaggtgttcccggaccccctggcgctgtcggtcctgctggcaaagatggagaggctggagctcagggaccccctggccctgctggtcccgctggcgagagaggtgaacaaggccctgctggctcccccggattccagggtctccctggtcctgctggtcctccaggtgaagcaggcaaacctggtgaacagggtgttcctggagaccttggcgcccctggcccctctggagcaagaggcgagagaggtttccctggcgagcgtggtgtgcaaggtccccctggtcctgctggtccccgaggggccaacggtgctcccggcaacgatggtgctaagggtgatgctggtgcccctggagctcccggtagccagggcgcccctggccttcagggaatgcctggtgaacgtggtgcagctggtcttccagggcctaagggtgacagaggtgatgctggtcccaaaggtgctgatggctctcctggcaaagatggcgtccgtggtctgactggccccattggtcctcctggccctgctggtgcccctggtgacaagggtgaaagtggtcccagcggccctgctggtcccactggagctcgtggtgcccccggagaccgtggtgagcctggtccccccggccctgctggctttgctggcccccctggtgctgacggccaacctggtgctaaaggcgaacctggtgatgctggtgctaaaggcgatgctggtccccctggccctgccggacccgctggaccccctggccccattggtaatgttggtgctcctggagccaaaggtgctcgcggcagcgctggtccccctggtgctactggtttccctggtgctgctggccgagtcggtcctcctggcccctctggaaatgctggaccccctggccctcctggtcctgctggcaaagaaggcggcaaaggtccccgtggtgagactggccctgctggacgtcctggtgaagttggtccccctggtccccctggccctgctggcgagaaaggatcccctggtgctgatggtcctgctggtgctcctggtactcccgggcctcaaggtattgctggacagcgtggtgtggtcggcctgcctggtcagagaggagagagaggcttccctggtcttcctggcccctctggtgaacctggcaaacaaggtccctctggagcaagtggtgaacgtggtccccctggtcccatgggcccccctggattggctggaccccctggtgaatctggacgtgagggggctcctggtgccgaaggttcccctggacgagacggttctcctggcgccaagggtgaccgtggtgagaccggccccgctggaccccctggtgctcctggtgctcctggtgcccctggccccgttggccctgctggcaagagtggtgatcgtggtgagactggtcctgctggtcccgccggtcctgtcggccctgttggcgcccgtggccccgccggaccccaaggcccccgtggtgacaagggtgagacaggcgaacagggcgacagaggcataaagggtcaccgtggcttctctggcctccagggtccccctggccctcctggctctcctggtgaacaaggtccctctggagcctctggtcctgctggtccccgaggtccccctggctctgctggtgctcctggcaaagatggactcaacggtctccctggccccattgggccccctggtcctcgcggtcgcactggtgatgctggtcctgttggtccccccggccctcctggacctcctggtccccctggtcctcccagcgctggtttcgacttcagcttcctgccccagccacctcaagagaaggctcacgatggtggccgctactaccgggctgatgatgccaatgtggttcgtgaccgtgacctcgaggtggacaccaccctcaagagcctgagccagcagatcgagaacatccggagcccagagggcagccgcaagaaccccgcccgcacctgccgtgacctcaagatgtgccactctgactggaagagtggagagtactggattgaccccaaccaaggctgcaacctggatgccatcaaagtcttctgcaacatggagactggtgagacctgcgtgtaccccactcagcccagtgtggcccagaagaactggtacatcagcaagaaccccaaggacaagaggcatgtctggttcggcgagagcatgaccgatggattccagttcgagtatggcggccagggctccgaccctgccgatgtggccatccagctgaccttcctgcgcctgatgtccaccgaggcctcccagaacatcacctaccactgcaagaacagcgtggcctacatggaccagcagactggcaacctcaagaaggccctgctcctccagggctccaacgagatcgagatccgcgccgagggcaacagccgcttcacctacagcgtcactgtcgatggctgcacgagtcacaccggagcctggggcaagacagtgattgaatacaaaaccaccaagacctcccgcctgcccatcatcgatgtggcccccttggacgttggtgccccagaccaggaattcggcttcgacgttggccctgtctgcttcctgtaaactccctccatcccaacctggctccctcccacccaaccaactttccccccaacccggaaacagacaagcaacccaaactgaaccccctcaaaagccaaaaaatgggagacaatttcacatggactttggaaaatatttttttcctttgcattcatctctcaaacttagtttttatctttgaccaaccgaacatgaccaaaaaccaaaagtgcattcaaccttaccaaaaaaaaaaaaaaaaaaagaataaataaataactttttaaaaaaggaagcttggtccacttgcttgaagacccatgcgggggtaagtccctttctgcccgttgggcttatgaaaccccaatgctgccctttctgctcctttctccacaccccccttggggcctcccctccactccttcccaaatctgtctccccagaagacacaggaaacaatgtattgtctgcccagcaatcaaaggcaatgctcaaacacccaagtggcccccaccctcagcccgctcctgcccgcccagcacccccaggccctgggggacctggggttctcagactgccaaagaagccttgccatctggcgctcccatggctcttgcaacatctccccttcgtttttgagggggtcatgccgggggagccaccagcccctcactgggttcggaggagagtcaggaagggccacgacaaagcagaaacatcggatttggggaacgcgtgtcaatcccttgtgccgcagggctgggcgggagagactgttctgttccttgtgtaactgtgttgctgaaagactacctcgttcttgtcttgatgtgtcaccggggcaactgcctgggggcggggatgggggcagggtggaagcggctccccattttataccaaaggtgctacatctatgtgatgggtggggtggggagggaatcactggtgctatagaaattgagatgcccccccaggccagcaaatgttcctttttgttcaaagtctatttttattccttgatatttttctttttttttttttttttttgtggatggggacttgtgaatttttctaaaggtgctatttaacatgggaggagagcgtgtgcggctccagcccagcccgctgctcactttccaccctctctccacctgcctctggcttctcaggcctctgctctccgacctctctcctctgaaaccctcctccacagctgcagcccatcctcccggctccctcctagtctgtcctgcgtcctctgtccccgggtttcagagacaacttcccaaagcacaaagcagtttttccccctaggggtgggaggaagcaaaagactctgtacctattttgtatgtgtataataatttgagatgtttttaattattttgattgctggaataaagcatgtggaaatgacccaaacataa

SEQ ID NO: 8 - DNA, 5411 bp, Homo sapiens
  source 1..5411: mol_type = genomic DNA
gtgtcccatagtgtttccaaacttggaaagggcgggggagggcgggaggatgcggagggcggaggtatgcagacaacgagtcagagtttccccttgaaagcctcaaaagtgtccacgtcctcaaaaagaatggaaccaatttaagaagccagccccgtggccacgtcccttcccccattcgctccctcctctgcgcccccgcaggctcctcccagctgtggctgcccgggcccccagccccagccctcccattggtggaggcccttttggaggcaccctagggccagggaaacttttgccgtataaatagggcagatccgggctttattattttagcaccacggcagcaggaggtttcggctaagttggaggtactggccacgactgcatgcccgcgcccgccaggtgatacctccgccggtgacccaggggctctgcgacacaaggagtctgcatgtctaagtgctagacatgctcagctttgtggatacgcggactttgttgctgcttgcagtaaccttatgcctagcaacatgccaatctttacaagaggaaactgtaagaaagggcccagccggagatagaggaccacgtggagaaaggggtccaccaggccccccaggcagagatggtgaagatggtcccacaggccctcctggtccacctggtcctcctggcccccctggtctcggtgggaactttgctgctcagtatgatggaaaaggagttggacttggccctggaccaatgggcttaatgggacctagaggcccacctggtgcagctggagccccaggccctcaaggtttccaaggacctgctggtgagcctggtgaacctggtcaaactggtcctgcaggtgctcgtggtccagctggccctcctggcaaggctggtgaagatggtcaccctggaaaacccggacgacctggtgagagaggagttgttggaccacagggtgctcgtggtttccctggaactcctggacttcctggcttcaaaggcattaggggacacaatggtctggatggattgaagggacagcccggtgctcctggtgtgaagggtgaacctggtgcccctggtgaaaatggaactccaggtcaaacaggagcccgtgggcttcctggtgagagaggacgtgttggtgcccctggcccagctggtgcccgtggcagtgatggaagtgtgggtcccgtgggtcctgctggtcccattgggtctgctggccctccaggcttcccaggtgcccctggccccaagggtgaaattggagctgttggtaacgctggtcctgctggtcccgccggtccccgtggtgaagtgggtcttccaggcctctccggccccgttggacctcctggtaatcctggagcaaacggccttactggtgccaagggtgctgctggccttcccggcgttgctggggctcccggcctccctggaccccgcggtattcctggccctgttggtgctgccggtgctactggtgccagaggacttgttggtgagcctggtccagctggctccaaaggagagagcggtaacaagggtgagcccggctctgctgggccccaaggtcctcctggtcccagtggtgaagaaggaaagagaggccctaatggggaagctggatctgccggccctccaggacctcctgggctgagaggtagtcctggttctcgtggtcttcctggagctgatggcagagctggcgtcatgggccctcctggtagtcgtggtgcaagtggccctgctggagtccgaggacctaatggagatgctggtcgccctggggagcctggtctcatgggacccagaggtcttcctggttcccctggaaatatcggccccgctggaaaagaaggtcctgtcggcctccctggcatcgacggcaggcctggcccaattggcccagctggagcaagaggagagcctggcaacattggattccctggacccaaaggccccactggtgatcctggcaaaaacggtgataaaggtcatgctggtcttgctggtgctcggggtgctccaggtcctgatggaaacaatggtgctcagggacctcctggaccacagggtgttcaaggtggaaaaggtgaacagggtccccctggtcctccaggcttccagggtctgcctggcccctcaggtcccgctggtgaagttggcaaaccaggagaaaggggtctccatggtgagtttggtctccctggtcctgctggtccaagaggggaacgcggtcccccaggtgagagtggtgctgccggtcctactggtcctattggaagccgaggtccttctggacccccagggcctgatggaaacaagggtgaacctggtgtggttggtgctgtgggcactgctggtccatctggtcctagtggactcccaggagagaggggtgctgctggcatacctggaggcaagggagaaaagggtgaacctggtctcagaggtgaaattggtaaccctggcagagatggtgctcgtggtgctcctggtgctgtaggtgcccctggtcctgctggagccacaggtgaccggggcgaagctggggctgctggtcctgctggtcctgctggtcctcggggaagccctggtgaacgtggtgaggtcggtcctgctggccccaatggatttgctggtcctgctggtgctgctggtcaacctggtgctaaaggagaaagaggagccaaagggcctaagggtgaaaacggtgttgttggtcccacaggccccgttggagctgctggcccagctggtccaaatggtccccccggtcctgctggaagtcgtggtgatggaggcccccctggtatgactggtttccctggtgctgctggacggactggtcccccaggaccctctggtatttctggccctcctggtccccctggtcctgctgggaaagaagggcttcgtggtcctcgtggtgaccaaggtccagttggccgaactggagaagtaggtgcagttggtccccctggcttcgctggtgagaagggtccctctggagaggctggtactgctggacctcctggcactccaggtcctcagggtcttcttggtgctcctggtattctgggtctccctggctcgagaggtgaacgtggtctaccaggtgttgctggtgctgtgggtgaacctggtcctcttggcattgccggccctcctggggcccgtggtcctcctggtgctgtgggtagtcctggagtcaacggtgctcctggtgaagctggtcgtgatggcaaccctgggaacgatggtcccccaggtcgcgatggtcaacccggacacaagggagagcgcggttaccctggcaatattggtcccgttggtgctgcaggtgcacctggtcctcatggccccgtgggtcctgctggcaaacatggaaaccgtggtgaaactggtccttctggtcctgttggtcctgctggtgctgttggcccaagaggtcctagtggcccacaaggcattcgtggcgataagggagagcccggtgaaaaggggcccagaggtcttcctggcttaaagggacacaatggattgcaaggtctgcctggtatcgctggtcaccatggtgatcaaggtgctcctggctccgtgggtcctgctggtcctaggggccctgctggtccttctggccctgctggaaaagatggtcgcactggacatcctggtacagttggacctgctggcattcgaggccctcagggtcaccaaggccctgctggcccccctggtccccctggccctcctggacctccaggtgtaagcggtggtggttatgactttggttacgatggagacttctacagggctgaccagcctcgctcagcaccttctctcagacccaaggactatgaagttgatgctactctgaagtctctcaacaaccagattgagacccttcttactcctgaaggctctagaaagaacccagctcgcacatgccgtgacttgagactcagccacccagagtggagcagtggttactactggattgaccctaaccaaggatgcactatggatgctatcaaagtatactgtgatttctctactggcgaaacctgtatccgggcccaacctgaaaacatcccagccaagaactggtataggagctccaaggacaagaaacacgtctggctaggagaaactatcaatgctggcagccagtttgaatataatgtagaaggagtgacttccaaggaaatggctacccaacttgccttcatgcgcctgctggccaactatgcctctcagaacatcacctaccactgcaagaacagcattgcatacatggatgaggagactggcaacctgaaaaaggctgtcattctacagggctctaatgatgttgaacttgttgctgagggcaacagcaggttcacttacactgttcttgtagatggctgctctaaaaagacaaatgaatggggaaagacaatcattgaatacaaaacaaataagccatcacgcctgcccttccttgatattgcacctttggacatcggtggtgctgaccaggaattctttgtggacattggcccagtctgtttcaaataaatgaactcaatctaaattaaaaaagaaagaaatttgaaaaaactttctctttgccatttcttcttcttcttttttaactgaaagctgaatccttccatttcttctgcacatctacttgcttaaattgtgggcaaaagagaaaaagaaggattgatcagagcattgtgcaatacagtttcattaactccttcccccgctcccccaaaaatttgaatttttttttcaacactcttacacctgttatggaaaatgtcaacctttgtaagaaaaccaaaataaaaattgaaaaataaaaaccataaacatttgcaccacttgtggcttttgaatatcttccacagagggaagtttaaaacccaaacttccaaaggtttaaactacctcaaaacactttcccatgagtgtgatccacattgttaggtgctgacctagacagagatgaactgaggtccttgttttgttttgttcataatacaaaggtgctaattaatagtatttcagatacttgaagaatgttgatggtgctagaagaatttgagaagaaatactcctgtattgagttgtatcgtgtggtgtattttttaaaaaatttgatttagcattcatattttccatcttattcccaattaaaagtatgcagattatttgcccaaatcttcttcagattcagcatttgttctttgccagtctcattttcatcttcttccatggttccacagaagctttgtttcttgggcaagcagaaaaattaaattgtacctattttgtatatgtgagatgtttaaataaattgtgaaaaaaatgaaataaagcatgtttggttttccaaaagaacatat

SEQ ID NO: 9 - DNA, 4467 bp, Homo sapiens
  source 1..4467: mol_type = genomic DNA
atggctcacgctcgtgttctcctcctcgctctcgctgttttggcaacagctgctgtggctgtggcttctagttcttcttttgctgattcaaaccctattagacctgttactgatagagcagcttccactttggctcaattgcaagaggagggccaggttgagggccaagatgaggatatccctccaattacatgcgtgcaaaatggcttgcgttaccacgatagggatgtgtggaaacctgaaccttgtcgtatctgtgtgtgtgataacggcaaggtgctctgcgatgatgttatctgcgatgagacaaaaaattgccctggcgctgaagttcctgagggcgagtgttgccctgtgtgccctgatggttccgagtccccaactgatcaggaaactactggcgtggagggcccaaaaggagatactggtccacgtggtcctaggggtccagcaggtcctccaggtagagatggtattccaggccagcctggattgccaggaccaccaggcccacctggcccaccaggacctcctggtcttggtggaaatttcgctccacaactctcttatggctatgatgagaagtcaacaggtggtatttccgttccaggtcctatgggaccatccggaccaagaggtctcccaggtcctccaggtgctcctggacctcaaggctttcaaggacctccaggcgaaccaggagaaccaggcgcttctggaccaatgggcccaaggggaccacctggcccaccaggaaaaaatggcgatgatggcgaagctggaaagcctggtcgtcctggagagagaggtcctcctggcccacagggtgcaagaggcttgccaggaactgctggcttgcctggaatgaagggacataggggcttctccggcctcgatggcgctaagggtgatgctggccctgctggaccaaagggcgagccaggttcccctggagaaaacggtgctcctggacaaatgggtcctcgtggacttccaggagaaaggggtcgtccaggcgctccaggaccagcaggtgctaggggaaacgatggtgcaacaggcgctgctggccctcctggcccaactggtcctgctggccctccaggattcccaggcgcagttggagctaaaggagaagcaggaccacagggccctaggggttctgaaggacctcagggtgttagaggtgaaccaggtcctccaggcccagctggagcagctggtccagcaggaaatccaggtgctgatggtcaacctggagctaagggcgctaatggcgcaccaggtatcgcaggcgcaccaggttttcctggcgctagaggcccaagtggtcctcaaggaccaggtggaccaccaggtccaaaaggcaattctggcgaacctggcgctccaggttctaaaggagatactggtgctaaaggcgaaccaggacctgttggtgttcagggtcctcctggtcctgctggagaagaaggaaaaagaggtgctcgtggagaaccaggaccaactggacttcctggacctcctggtgaacgtggcggacctggctcaaggggtttccctggagctgatggagtggcaggtccaaaaggccctgctggagagagaggttcaccaggtccagctggtcctaagggctcccctggtgaagcaggtagaccaggcgaagcaggattgccaggcgcaaagggattgacaggctctcctggtagtcctggcccagatggaaaaacaggcccaccaggtccagcaggacaagatggacgtccaggcccaccaggtcctcctggagcaaggggacaagctggcgttatgggttttccaggacctaaaggtgctgctggagagccaggaaaggcaggtgaaagaggagttcctggtccaccaggagcagtgggtcctgctggcaaagatggtgaagctggagcacagggccctccaggccctgctggcccagctggcgaacgtggagaacaaggcccagctggtagtccaggatttcaaggattgcctggccctgctggccctccaggagaagcaggaaaacctggagaacaaggagttcctggtgatttgggagcacctggaccttcaggagcacgtggtgaaagaggcttccctggcgagaggggtgttcaaggtccaccaggtccagcaggacctagaggtgctaatggcgctcctggcaacgatggagcaaaaggtgatgctggtgctcctggcgcacctggaagtcagggtgctcctggattgcaaggaatgcctggagagaggggtgctgctggcttgccaggcccaaagggcgataggggtgatgctggaccaaaaggtgctgatggatccccaggaaaagatggagttcgtggtcttactggcccaatcggacctccaggccctgctggcgctccaggtgataagggcgaaagtggcccaagtggacctgctggacctactggtgctagaggtgcacctggtgataggggtgaacctggaccacctggtccagctggttttgctggtcctcctggagctgatggacaacctggcgcaaagggtgaaccaggtgatgctggcgcaaagggagatgctggtccacctggacctgctggtccagcaggcccccctgggccaatcggtaatgttggagcaccaggtgctaagggagctaggggttccgctggtccacctggagcaacaggatttccaggcgctgctggtagagttggcccaccaggcccatccggaaacgcaggccctcctggtcctccaggtcctgctggcaaggagggtggcaaaggaccaaggggcgaaactggccctgctggtagacctggcgaagttggccctcctggaccaccaggtccagcaggagaaaaaggttccccaggagctgatggcccagctggtgctccaggaactccaggccctcaaggtattgctggacagagaggcgttgtgggactccctggtcaaaggggagagagaggatttccaggcttgccaggacctagtggagaacctggaaaacaaggcccatcaggcgctagtggagagcgtggacctcctggccctatgggacctcctggattggctggcccacctggcgaatcaggtcgtgaaggcgcaccaggcgcagaaggatcacctggaagagatggatcccctggtgctaaaggcgatcgtggagaaactggtccagcaggcccaccaggcgcaccaggtgcacctggcgctccaggacctgtgggaccagctggaaaatccggagataggggcgagacaggcccagcaggaccagctggacctgttggccctgctggcgctcgtggaccagcaggacctcaaggaccaaggggagataagggagaaacaggcgaacaaggcgataggggcattaagggtcataggggttttagtggcctccagggtcctcctggcccacctggatcaccaggagaacagggaccatctggtgcttccggcccagctggtccaagaggacctccaggatcagctggtgcacctggaaaagatggtcttaacggtctcccaggaccaatcggccctccaggacctagaggaagaacaggagatgctggccctgttggccctccaggacctcctggtccaccaggtccacctggtcctccatcagctggattcgatttttcatttcttccacagccaccacaagagaaagctcacgatggcggcagatattaccgtgctgatgatgctaacgttgttagggatagagatttggaagtggatacaactttgaaatccctctcccagcaaattgaaaacattagatctccagaaggttcacgtaaaaacccagctagaacatgtcgtgatttgaaaatgtgtcactccgattggaaaagtggtgaatactggattgatccaaatcagggctgtaatctcgatgctatcaaagttttctgtaacatggaaacaggcgaaacatgcgtttatcctactcaaccttccgtggctcagaaaaattggtacatctcaaaaaatcctaaagataagaggcacgtttggttcggtgaaagtatgactgatggatttcaatttgagtacggcggtcaaggtagtgatccagctgatgtggctattcaactcacatttttgcgtcttatgtccacagaggcatcacaaaacatcacttaccactgcaaaaacagtgtggcttatatggatcaacaaacaggaaaccttaagaaggctcttcttttgaagggctcaaacgagattgagattagagcagagggcaactcaaggtttacttattcagttactgttgatggctgcacttcacatactggcgcttggggtaaaacagttatcgagtataagactacaaaaacatcaagactcccaatcattgatgttgctcctctcgatgttggcgctcctgatcaagagttcggttttgatgtgggcccagtttgtttcctc

SEQ ID NO: 10 - DNA, 4167 bp, Homo sapiens
  source 1..4167: mol_type = genomic DNA
atggctcacgctcgtgttctcctcctcgctctcgctgttttggcaacagctgctgtggctgtggcttcaagttctagttttgctgattccaacccaattcgtccagttactgatagagcagcttccactttggctcaattgcttcaagaagaaactgtgaggaagggccctgctggcgataggggccctaggggcgaaaggggtccaccaggacctccaggcagggatggcgaagatggtccaactggccctcctggacctcctggccctccagggccacccggcttgggcggaaacttcgcagctcaatacgatggcaagggtgttggtcttggtcctggtcctatgggcttgatgggacctagaggcccacctggtgctgctggtgctcctggaccacagggttttcagggaccagctggcgagccaggagagccaggccaaacaggaccagctggtgcaaggggacctgctggacctcctggaaaagctggtgaagatggtcacccaggcaaaccaggacgtcctggcgaaagaggtgttgttggaccacaaggcgctaggggatttccaggtacacctggattgccaggttttaagggcattcgtggtcataacggcctcgatggattgaagggacagcctggcgcacctggcgttaagggtgaacctggagcaccaggtgaaaacggtactcctggccagactggtgcaagaggactcccaggtgaaaggggtagagttggtgctcctggacctgctggagctaggggtagtgatggtagtgttggtcctgtgggccctgctggtccaatcggttccgctggcccacctggattcccaggcgctccaggacctaaaggagaaatcggtgctgtgggtaacgcaggtcctactggtccagcaggtcctcgtggagaagtgggattgccaggactttctggtccagtgggccctccaggcaaccctggagctaacggcttgacaggagctaaaggcgcagcaggactccctggagtggctggcgcaccaggattgcctggtccaaggggtatcccaggccctgttggcgcagctggagctactggtgcacgtggacttgttggcgaaccaggccctgctggatcaaaaggcgagtctggaaataagggagaacctggttctgctggacctcaaggtcctcctggaccttctggagaagaaggaaaaaggggaccaaatggcgaggctggatcagcaggtccaccaggaccacctggacttcgtggatcccctggtagtagaggacttccaggcgctgatggtagagcaggcgttatgggaccaccaggaagtagaggagcatccggtccagcaggagttaggggtcctaacggagatgctggtagaccaggtgaaccaggtcttatgggcccaaggggcctcccaggtagtccaggaaatatcggccctgctggaaaagaaggccctgttggacttccaggtattgatggacgtcctggccctattggcccagcaggtgcaagaggagaacctggcaatattggatttccaggaccaaagggtccaacaggcgatcctggaaaaaatggagataagggtcatgctggattggcaggcgcaaggggcgctcctggtccagatggaaacaacggcgcacagggtccacctggccctcagggtgttcaaggcggaaaaggcgaacaaggcccagctggaccaccaggctttcaaggcttgccaggaccaagtggtccagcaggtgaagttggcaagccaggcgagcgtggacttcatggcgagtttggactccctggaccagcaggaccaaggggtgaaagaggccctcctggagagagtggcgctgctggaccaacaggcccaatcggtagtagaggtcctagtggacctccaggcccagatggaaataagggtgaaccaggagttgtgggcgctgttggaacagctggtccttcaggaccatcaggactcccaggcgagagaggcgctgctggcattcctggaggaaaaggtgaaaaaggcgaacctggcctccgtggcgaaatcggaaatcctggacgtgatggtgctcgtggtgcacacggcgctgtgggcgctccaggccctgctggtgctactggtgatagaggagaggctggcgcagctggcccagcaggtcctgctggcccaaggggtagtcctggtgaaagaggcgaagttggacctgctggccctaacggctttgctggccctgctggagcagcaggtcaacctggcgctaaaggtgaaaggggcggaaagggcccaaaaggtgaaaatggcgttgtgggaccaactggtccagtgggcgcagctggacctgctggtccaaatggaccaccaggaccagcaggtagtagaggagatggtggacctccaggaatgacaggttttccaggtgctgctggtagaacaggacctcctggtcctagtggtatttctggtccaccaggaccaccaggtcctgctggaaaagaaggattgaggggtccacgtggtgatcaaggaccagtgggcagaactggtgaagttggcgcagtgggaccacctggttttgctggagaaaagggcccttctggagaggcaggaacagctggtcctcctggtacacctggacctcaaggacttttgggtgcacctggtattctcggattgccaggaagtaggggcgaacgtggacttcctggcgtggcaggagcagttggagaacctggccctctcggaatcgcaggcccaccaggcgcaagaggaccaccaggagctgttggatcaccaggcgtgaatggtgcacctggcgaggctggtcgtgatggaaacccaggaaatgatggcccaccaggaagagatggtcaacctggacacaaaggcgagaggggctacccaggaaatattggcccagttggtgctgctggcgcaccaggcccacacggtccagttggaccagcaggaaaacacggtaatcgtggcgaaacaggcccttcaggcccagtgggacctgctggtgctgttggcccaagaggaccatctggacctcaaggcattagaggcgataagggagagcctggcgaaaaaggacctagaggcttgcctggttttaaaggacacaacggtctccaaggacttccaggtatcgctggtcatcatggagatcagggtgctcctggatcagtgggtccagcaggtcctagaggcccagcaggcccttccggtccagcaggaaaggatggacgtactggccaccctggaactgtgggccctgctggaattagaggtcctcaaggtcatcagggccctgctggccctccaggtccaccaggtcctccaggcccaccaggagtttcaggtggtggttacgattttggttacgatggtgatttttaccgtgctgatcaacctagaagtgctccttctctccgtcctaaagattatgaagttgatgctactttgaaatcacttaacaaccagattgagactcttctcacacctgagggatcaagaaagaatccagcacgtacatgccgtgatctcagacttagtcacccagagtggtcaagtggctattattggattgatcctaatcagggttgtacaatggaggctatcaaagtttactgtgattttccaactggagagacatgtattagggcacaacctgagaacattccagctaaaaattggtatcgttcctctaaagataagaaacatgtttggctcggagagactattaacgctggttctcagttcgagtataatgttgagggcgttacttctaaagagatggcaactcagctcgcttttatgagattgctcgctaactacgcatcccaaaacatcacttatcactgcaaaaattccattgcatatatggatgaggagacaggaaatttgaagaaagcagttattctccaaggtagtaacgatgttgagcttgtggctgagggaaatagtagattcacttacacagttttggtggatggatgctcaaagaaaactaatgagtggggcaagacaatcattgagtacaagacaaataagccttctaggctcccatttctcgatattgcacctcttgatatcggaggagctgatcacgagttttttgttgatatcggacctgtttgttttaag

SEQ ID NO: 11 - DNA, 1633 bp, synthetic construct
  misc_feature 1..1633: note = Synthetic sequence containing the coding regions of the vascular signal sequence of barley gene for Thiol protease aleurain precursor fused to the human Prolyl 4-hydroxylase beta subunitand flanking regions
  source 1..1633: mol_type = other DNA
ctcgagtaaaccatggctcatgctagggttttgcttttggctcttgctgttcttgctactgctgctgttgctgtggcttcttcttcatctttcgctgattctaacccaattaggccagtgactgatagagctgcttctactcttgctcaattggtcgacatggatgctccagaagaggaggatcacgttcttgtgcttaggaagtctaacttcgctgaagctcttgctgctcacaagtaccttcttgtggagttttatgctccttggtgcggacattgcaaagctcttgctccagagtatgctaaggctgctggaaagttgaaggctgagggatctgaaattaggcttgctaaagtggatgctactgaggagtctgatcttgctcaacagtacggagttaggggatacccaactattaagttcttcaggaacggagatactgcttctccaaaggagtatactgctggaagggaggctgatgatattgtgaactggcttaagaagagaactggaccagctgctactactcttccagatggagctgctgctgaatctcttgtggagtcatctgaggtggcagtgattggattcttcaaggatgtggagtctgattctgctaagcagttccttcaagctgctgaggctattgatgatattccattcggaattacttctaactctgatgtgttctctaagtaccagcttgataaggatggagtggtgcttttcaagaaattcgatgagggaaggaacaatttcgagggagaggtgacaaaggagaaccttcttgatttcattaagcacaaccagcttccacttgtgattgagttcactgagcagactgctccaaagattttcggaggagagattaagactcacattcttcttttccttccaaagtctgtgtctgattacgatggaaagttgtctaacttcaagactgctgctgagtctttcaagggaaagattcttttcattttcattgattctgatcacactgataaccagaggattcttgagttcttcggacttaagaaggaagagtgcccagctgttaggcttattactcttgaggaggagatgactaagtacaagccagagtctgaagaacttactgctgagaggattactgagttctgccacagattccttgagggaaagattaagccacaccttatgtctcaagagcttccagaggattgggataagcagccagttaaggtgttggtgggtaaaaacttcgaggatgtggctttcgatgagaagaagaacgtgttcgtggagttctacgcaccttggtgtggtcactgtaagcagcttgctccaatttgggataagttgggagagacttacaaggatcacgagaacattgtgattgctaagatggattctactgctaacgaggtggaggctgttaaggttcactctttcccaactttgaagttcttcccagcttctgctgataggactgtgattgattacaacggagaaaggactcttgatggattcaagaagttccttgagtctggaggacaagatggagctggagatgatgatgatcttgaggatttggaagaagctgaggagccagatatggaggaggatgatgatcagaaggctgtgtgatgagctc

SEQ ID NO: 12 - DNA, 1723 bp, synthetic construct
  misc_feature 1..1723: note = Synthetic sequence containing the coding regions of the vascular signal sequence of barley gene for Thiol protease aleurain precursor fused to the human Prolyl 4-hydroxylase alpha-1 subunitand flanking regions
  source 1..1723: mol_type = other DNA
ctcgagtaaaccatggctcatgctagggttttgcttttggctcttgctgttcttgctactgctgctgttgctgtggcttcttcttcatctttcgctgattctaacccaattaggccagtgactgatagagctgcttctactcttgctcaattggtcgacatgcacccaggattcttcacttctattggacagatgactgatcttattcacactgagaaggatcttgtgacttctcttaaggattacattaaggctgaggaggataagttggagcagattaagaagtgggctgagaagttggataggcttacttctactgctacaaaagatccagagggattcgttggtcatccagtgaacgctttcaagttgatgaagaggcttaacactgagtggagtgagcttgagaaccttgtgcttaaggatatgtctgatggattcatttctaaccttactattcagaggcagtacttcccaaatgatgaggatcaagtgggagctgctaaggctcttcttaggcttcaggatacttacaaccttgatactgatacaatttctaagggaaaccttccaggagttaagcacaagtctttccttactgctgaggattgcttcgagcttggaaaggttgcatacactgaggctgattactaccacactgagctttggatggaacaagctcttaggcaacttgatgagggagagatttctactattgataaggtgtcagtgcttgattacctttcttacgctgtgtaccagcagggtgatcttgataaggctcttttgcttactaagaagttgcttgagcttgatccagaacatcagagggctaacggaaaccttaagtacttcgagtacattatggctaaggaaaaggatgtgaacaagtctgcttctgatgatcagtctgatcaaaagactactccaaagaagaagggagtggctgttgattatcttcctgagaggcagaagtatgagatgttgtgtaggggagagggtattaagatgactccaaggaggcagaagaagttgttctgcaggtatcacgatggaaacaggaacccaaagttcattcttgctccagctaagcaagaagatgagtgggataagccaaggattattaggttccacgatattatttctgatgctgagattgagattgtgaaggatcttgctaagccaagacttaggagggctactatttctaaccctattactggtgatcttgagactgtgcactacaggatttctaagtctgcttggctttctggatacgagaacccagtggtgtctaggattaacatgaggattcaggatcttactggacttgatgtgtctactgctgaggagcttcaagttgctaactacggagttggaggacaatatgagccacacttcgatttcgctaggaaggatgagccagatgcttttaaggagcttggaactggaaacaggattgctacttggcttttctacatgtctgatgtttctgctggaggagctactgttttcccagaagtgggagcttctgtttggccaaagaagggaactgctgtgttctggtacaaccttttcgcttctggagagggagattactctactaggcatgctgcttgcccagttcttgttggaaacaagtgggtgtcaaacaagtggcttcatgagaggggacaagagtttagaaggccatgcactctttctgagcttgagtgatgagctc

SEQ ID NO: 13 - DNA, 2888 bp, synthetic construct
  misc_feature 1..2888: note = Synthetic sequence containing the coding regions of the vascular signal sequence of barley gene for Thiol protease aleurain precursor fused to the human Lysyl hydroxylase 3 and flankingregions
  source 1..2888: mol_type = other DNA
gcgaattcgctagctatcactgaaaagacagcaagacaatggtgtctcgatgcaccagaaccacatctttgcagcagatgtgaagcagccagagtggtccacaagacgcactcagaaaaggcatcttctaccgacacagaaaaagacaaccacagctcatcatccaacatgtagactgtcgttatgcgtcggctgaagataagactgaccccaggccagcactaaagaagaaataatgcaagtggtcctagctccactttagctttaataattatgtttcattattattctctgcttttgctctctatataaagagcttgtattttcatttgaaggcagaggcgaacacacacacagaacctccctgcttacaaaccagatcttaaaccatggctcacgctagggttttgcttcttgctcttgctgttcttgctactgctgctgttgctgtggcttcttcaagttctttcgctgattctaacccaattaggccagtgactgatagagctgcttctactcttgctcaattgagatctatgtctgatagaccaaggggaagggatccagttaatccagagaagttgcttgtgattactgtggctactgctgagactgaaggataccttagattccttaggagtgctgagttcttcaactacactgtgaggactcttggacttggagaagaatggaggggaggagatgttgctagaactgttggaggaggacagaaagtgagatggcttaagaaagagatggagaagtacgctgatagggaggatatgattattatgttcgtggattcttacgatgtgattcttgctggatctccaactgagcttttgaagaaattcgttcagtctggatctaggcttcttttctctgctgagtctttttgttggccagaatggggacttgctgagcaatatccagaagtgggaactggaaagagattccttaactctggaggattcattggattcgctactactattcaccagattgtgaggcagtggaagtacaaggatgacgatgatgatcagcttttctacactaggctttaccttgatccaggacttagggagaagttgtctcttaaccttgatcacaagtctaggattttccagaaccttaacggtgctcttgatgaggttgtgcttaagttcgataggaacagagtgaggattaggaacgtggcttacgatactcttcctattgtggtgcatggaaacggaccaacaaaactccagcttaactaccttggaaactacgttccaaacggatggactccagaaggaggatgtggattctgcaatcaggataggagaactcttccaggaggacaaccaccaccaagagttttccttgctgtgttcgttgaacagccaactccattccttccaagattccttcagaggcttcttcttttggattacccaccagatagggtgacacttttccttcacaacaacgaggttttccacgagccacacattgctgattcttggccacagcttcaggatcatttctctgctgtgaagttggttggtccagaagaagctctttctccaggagaagctagggatatggctatggatttgtgcaggcaggatccagagtgcgagttctacttctctcttgatgctgatgctgtgcttactaaccttcagactcttaggattcttattgaggagaacaggaaagtgattgctccaatgctttctaggcacggaaagttgtggtctaatttctggggtgctctttctcctgatgagtactacgctagatcagaggactacgtggagcttgttcagagaaagagagtgggagtttggaacgttccttatatttctcaggcttacgtgattaggggagatactcttaggatggagcttccacagagggatgttttctctggatctgatactgatccagatatggctttctgcaagtctttcagggataagggaattttccttcacctttctaaccagcatgagttcggaagattgcttgctacttcaagatacgatactgagcaccttcatcctgatctttggcagattttcgataacccagtggattggaaggagcagtacattcacgagaactactctagggctcttgaaggagaaggaattgtggagcaaccatgcccagatgtttactggttcccacttctttctgagcaaatgtgcgatgagcttgttgctgagatggagcattacggacaatggagtggaggtagacatgaggattctaggcttgctggaggatacgagaacgttccaactgtggatattcacatgaagcaagtgggatacgaggatcaatggcttcagcttcttaggacttatgtgggaccaatgactgagtctcttttcccaggataccacactaaggctagggctgttatgaacttcgttgtgaggtatcgtccagatgagcaaccatctcttaggccacaccacgattcttctactttcactcttaacgtggctcttaaccacaagggacttgattatgagggaggaggatgccgtttccttagatacgattgcgtgatttcttcaccaagaaagggatgggctcttcttcatccaggaaggcttactcattaccacgagggacttccaactacttggggaactagatatattatggtgtctttcgtggatccatgactgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccagaattcgcg

SEQ ID NO: 14 - protein, 45 aa, synthetic construct
  REGION 1..45: note = Vacuolar targeting sequence of the thiol protease aleurain precursor (NCBI accession P05167GI:113603)
MAHARVLLLALAVLATAAVAVASSSSFADSNPIRPVTDRAASTLA

SEQ ID NO: 15 - protein, 1469 aa, Homo sapiens
MAHARVLLLALAVLATAAVAVASSSSFADSNPIRPVTDRAASTLAQLQEEGQVEGQDEDIPPITCVQNGLRYHDRDVWKPEPCRICVCDNGKVLCDDVICDETKNCPGAEVPEGECCPVCPDGSESPTDQETTGVEGPKGDTGPRGPRGPAGPPGRDGIPGQPGLPGPPGPPGPPGPPGLGGNFAPQLSYGYDEKSTGGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEAGPQGPRGSEGPQGVRGEPGPPGPAGAAGPAGNPGADGQPGAKGANGAPGIAGAPGFPGARGPSGPQGPGGPPGPKGNSGEPGAPGSKGDTGAKGEPGPVGVQGPPGPAGEEGKRGARGEPGPTGLPGPPGERGGPGSRGFPGADGVAGPKGPAGERGSPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGSPGKDGVRGLTGPIGPPGPAGAPGDKGESGPSGPAGPTGARGAPGDRGEPGPPGPAGFAGPPGADGQPGAKGEPGDAGAKGDAGPPGPAGPAGPPGPIGNVGAPGAKGARGSAGPPGATGFPGAAGRVGPPGPSGNAGPPGPPGPAGKEGGKGPRGETGPAGRPGEVGPPGPPGPAGEKGGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPVGPAGARGPAGPQGPRGDKGETGEQGDRGIKGHRGFSGLQGPPGPPGSPGEQGPSGASGPAGPRGPPGSAGAPGKDGLNGLPGPIGPPGPRGRTGDAGPVGPPGPPGPPGPPGPPSAGFDFSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKTSRLPIIDVAPLDVGAPDQEFGFDVGPVCFL

SEQ ID NO: 16 - DNA, 4416 bp, Homo sapiens
  source 1..4416: mol_type = genomic DNA
atggctcacgctcgtgttctcctcctcgctctcgctgttttggcaacagctgctgtggctgtggcttctagttcttcttttgctgattcaaaccctattagacctgttactgatagagcagcttccactttggctcaattgcaagaggagggccaggttgagggccaagatgaggatatccctccaattacatgcgtgcaaaatggcttgcgttaccacgatagggatgtgtggaaacctgaaccttgtcgtatctgtgtgtgtgataacggcaaggtgctctgcgatgatgttatctgcgatgagacaaaaaattgccctggcgctgaagttcctgagggcgagtgttgccctgtgtgccctgatggttccgagtccccaactgatcaggaaactactggcgtggagggcccaaaaggagatactggtccacgtggtcctaggggtccagcaggtcctccaggtagagatggtattccaggccagcctggattgccaggaccaccaggcccacctggcccaccaggacctcctggtcttggtggaaatttcgctccacaactctcttatggctatgatgagaagtcaacaggtggtatttccgttccaggtcctatgggaccatccggaccaagaggtctcccaggtcctccaggtgctcctggacctcaaggctttcaaggacctccaggcgaaccaggagaaccaggcgcttctggaccaatgggcccaaggggaccacctggcccaccaggaaaaaatggcgatgatggcgaagctggaaagcctggtcgtcctggagagagaggtcctcctggcccacagggtgcaagaggcttgccaggaactgctggcttgcctggaatgaagggacataggggcttctccggcctcgatggcgctaagggtgatgctggccctgctggaccaaagggcgagccaggttcccctggagaaaacggtgctcctggacaaatgggtcctcgtggacttccaggagaaaggggtcgtccaggcgctccaggaccagcaggtgctaggggaaacgatggtgcaacaggcgctgctggccctcctggcccaactggtcctgctggccctccaggattcccaggcgcagttggagctaaaggagaagcaggaccacagggccctaggggttctgaaggacctcagggtgttagaggtgaaccaggtcctccaggcccagctggagcagctggtccagcaggaaatccaggtgctgatggtcaacctggagctaagggcgctaatggcgcaccaggtatcgcaggcgcaccaggttttcctggcgctagaggcccaagtggtcctcaaggaccaggtggaccaccaggtccaaaaggcaattctggcgaacctggcgctccaggttctaaaggagatactggtgctaaaggcgaaccaggacctgttggtgttcagggtcctcctggtcctgctggagaagaaggaaaaagaggtgctcgtggagaaccaggaccaactggacttcctggacctcctggtgaacgtggcggacctggctcaaggggtttccctggagctgatggagtggcaggtccaaaaggccctgctggagagagaggttcaccaggtccagctggtcctaagggctcccctggtgaagcaggtagaccaggcgaagcaggattgccaggcgcaaagggattgacaggctctcctggtagtcctggcccagatggaaaaacaggcccaccaggtccagcaggacaagatggacgtccaggcccaccaggtcctcctggagcaaggggacaagctggcgttatgggttttccaggacctaaaggtgctgctggagagccaggaaaggcaggtgaaagaggagttcctggtccaccaggagcagtgggtcctgctggcaaagatggtgaagctggagcacagggccctccaggccctgctggcccagctggcgaacgtggagaacaaggcccagctggtagtccaggatttcaaggattgcctggccctgctggccctccaggagaagcaggaaaacctggagaacaaggagttcctggtgatttgggagcacctggaccttcaggagcacgtggtgaaagaggcttccctggcgagaggggtgttcaaggtccaccaggtccagcaggacctagaggtgctaatggcgctcctggcaacgatggagcaaaaggtgatgctggtgctcctggcgcacctggaagtcagggtgctcctggattgcaaggaatgcctggagagaggggtgctgctggcttgccaggcccaaagggcgataggggtgatgctggaccaaaaggtgctgatggatccccaggaaaagatggagttcgtggtcttactggcccaatcggacctccaggccctgctggcgctccaggtgataagggcgaaagtggcccaagtggacctgctggacctactggtgctagaggtgcacctggtgataggggtgaacctggaccacctggtccagctggttttgctggtcctcctggagctgatggacaacctggcgcaaagggtgaaccaggtgatgctggcgcaaagggagatgctggtccacctggacctgctggtccagcaggcccccctgggccaatcggtaatgttggagcaccaggtgctaagggagctaggggttccgctggtccacctggagcaacaggatttccaggcgctgctggtagagttggcccaccaggcccatccggaaacgcaggccctcctggtcctccaggtcctgctggcaaggagggtggcaaaggaccaaggggcgaaactggccctgctggtagacctggcgaagttggccctcctggaccaccaggtccagcaggagaaaaaggtggacagagaggcgttgtgggactccctggtcaaaggggagagagaggatttccaggcttgccaggacctagtggagaacctggaaaacaaggcccatcaggcgctagtggagagcgtggacctcctggccctatgggacctcctggattggctggcccacctggcgaatcaggtcgtgaaggcgcaccaggcgcagaaggatcacctggaagagatggatcccctggtgctaaaggcgatcgtggagaaactggtccagcaggcccaccaggcgcaccaggtgcacctggcgctccaggacctgtgggaccagctggaaaatccggagataggggcgagacaggcccagcaggaccagctggacctgttggccctgctggcgctcgtggaccagcaggacctcaaggaccaaggggagataagggagaaacaggcgaacaaggcgataggggcattaagggtcataggggttttagtggcctccagggtcctcctggcccacctggatcaccaggagaacagggaccatctggtgcttccggcccagctggtccaagaggacctccaggatcagctggtgcacctggaaaagatggtcttaacggtctcccaggaccaatcggccctccaggacctagaggaagaacaggagatgctggccctgttggccctccaggacctcctggtccaccaggtccacctggtcctccatcagctggattcgatttttcatttcttccacagccaccacaagagaaagctcacgatggcggcagatattaccgtgctgatgatgctaacgttgttagggatagagatttggaagtggatacaactttgaaatccctctcccagcaaattgaaaacattagatctccagaaggttcacgtaaaaacccagctagaacatgtcgtgatttgaaaatgtgtcactccgattggaaaagtggtgaatactggattgatccaaatcagggctgtaatctcgatgctatcaaagttttctgtaacatggaaacaggcgaaacatgcgtttatcctactcaaccttccgtggctcagaaaaattggtacatctcaaaaaatcctaaagataagaggcacgtttggttcggtgaaagtatgactgatggatttcaatttgagtacggcggtcaaggtagtgatccagctgatgtggctattcaactcacatttttgcgtcttatgtccacagaggcatcacaaaacatcacttaccactgcaaaaacagtgtggcttatatggatcaacaaacaggaaaccttaagaaggctcttcttttgaagggctcaaacgagattgagattagagcagagggcaactcaaggtttacttattcagttactgttgatggctgcacttcacatactggcgcttggggtaaaacagttatcgagtataagactacaaaaacatcaagactcccaatcattgatgttgctcctctcgatgttggcgctcctgatcaagagttcggttttgatgtgggcccagtttgtttcctctaatgagct